(19) United States
(12) Patent Application Publication
(10) Pub. No.: US 2024/0347130 A1
(43) Pub. Date: Oct. 17, 2024

(54) SYSTEMS AND METHODS FOR DISCOVERING COMPOUNDS USING CAUSAL INFERENCE

(71) Applicant: DeepCure Inc., Boston, MA (US)

(72) Inventors: Derek Miller, Norfolk, MA (US); Jonathan Kaufman, Malden, MA (US)

(21) Appl. No.: 18/634,679

(22) Filed: Apr. 12, 2024

Related U.S. Application Data
(60) Provisional application No. 63/495,926, filed on Apr. 13, 2023.

(51) Int. Cl. G16B 15/30; G16B 40/20

(52) U.S. Cl. CPC G16B 15/30; G16B 40/20

(57) ABSTRACT

Systems and methods for characterizing interactions between molecules and targets are provided. Candidate molecules are selected using first interaction scores for each molecule and a target. Molecular graphs of each molecule are inputted into a first and/or second model to retrieve a first plurality of on-target interaction features or a second plurality of off-target interaction features, respectively. Second interaction scores are obtained using the first and/or second plurality of features and evaluated to filter the plurality of candidate molecules. For each molecule, a third plurality of binding affinity interaction features and/or a fourth plurality of specificity interaction features are determined. The plurality of candidate molecules is filtered based at least on counts of features in the third and/or fourth plurality of features. Predictions of interaction between each molecule and the target are determined using at least the third and/or fourth plurality of features.

### CROSS REFERENCE TO RELATED APPLICATIONS

This application claims priority to U.S. Provisional Patent Application Ser. No. 63/495,926, filed Apr. 13, 2023, which is hereby incorporated by reference in its entirety.

### TECHNICAL FIELD

This application is directed to using interaction features to characterize interactions between candidate molecules and target macromolecules or macromolecule complex.

### BACKGROUND

Pharmaceutical companies spend millions of dollars screening compounds to discover novel compounds and develop them into prospective drug leads. Traditionally, this has involved collecting large libraries of compounds tested to find the small number of compounds that interact with the disease target of interest. Unfortunately, gathering these large screening collections imposes significant challenges through storage constraints, shelf stability, or chemical cost. Furthermore, the cost and time needed to physically assay of compounds is prohibitive to testing them at scale. Even the largest pharmaceutical companies are testing only hundreds of thousands to a few millions of compounds at a time, versus the tens of millions of commercially available compounds and the billions, and even trillions of compounds that can be generated and screened computationally.

One key characteristic of a successful drug candidate is strong binding against its disease target. However, compounds that bind strongly enough to be clinically effective are rare.

Approximately half of the drug candidates in late-stage clinical trials fail due to unacceptable toxicity. Toxicity can be due to off-target side effects caused by a compound binding non-selectively to other targets. Therefore, increasing potent binding to the desired target while decreasing non-selective binding to other related targets is important in drug discovery. Drug candidates can also fail because they do not have desirable pharmacological absorption, distribution, metabolic, and excretion (ADME) profiles. Optimizing and balancing multiple objectives such as potency, selectivity, toxicity, and pharmacological properties is challenging but essential for a compound to become a drug.

Due to the many requirements for a compound to be a drug, there is a need to explore large and diverse chemical spaces of compounds that have different interactions with the target and, therefore, different properties. Large and diverse libraries of compounds also increase the odds of finding compounds that simultaneously satisfy all the other ADME properties needed to be a safe and effective drug. Thus, a better method is needed to accurately, rapidly, and efficiently identify or generate compounds that interact with the desired target (and do not interact with other targets), as measured by the Gibbs free energy.

Given the above background, what is needed in the art are methods for designing, identifying, and/or generating candidate compounds having target interaction properties when complexed with target macromolecules.

### SUMMARY

The present disclosure addresses the problems identified in the background by providing systems and methods that make use of hypothesis generation procedures and machine learning models to predict interaction features between candidate molecules and target macromolecules or macromolecule complexes. The disclosed systems and methods utilize a causal-based method that predicts the underlying causal interactions a molecular conformation makes with a target macromolecule, rather than directly predicting binding affinity or specificity. In some implementations, the disclosed systems and methods further utilize causal inference rather than correlation to account for and reduce the confounding effect of certain biases. As detailed in Example 1 below, the disclosed systems and methods improve lead compound identification at a higher hit rate, potency, selectivity, and cost-efficiency compared to conventional methods. Advantageously, in some implementations, the disclosed systems and methods are used to generate hundreds of thousands of compounds based on robust reactions from a large exploration space that have a high probability of binding, a desired selectivity profile, and carry few or no ADME liabilities.

Accordingly, one aspect of the present disclosure provides a method for characterizing an interaction between a candidate molecule and a target macromolecule or a target macromolecule complex. In some embodiments, the method is performed at a computer system comprising one or more processing cores and a memory. In some embodiments, the method includes selecting a plurality of candidate molecules from a collection of candidate molecules based on a respective first score for the interaction between each respective candidate molecule in the collection of candidate molecules and the target macromolecule or the target macromolecule complex, where the plurality of candidate molecules comprises at least 1×10⁶ candidate molecules.

In some embodiments, a first filtering step is performed for the plurality of candidate molecules comprising, for each respective candidate molecule in the plurality of candidate molecules, responsive to inputting a two-dimensional molecular graph of the respective candidate molecule into a first model, retrieving, as output from the first model, a corresponding first plurality of interaction features for a complex formed between the respective candidate molecule and the target macromolecule or the target macromolecule complex. Alternatively or additionally, in some embodiments, responsive to inputting the two-dimensional molecular graph of the respective candidate molecule into a second model, the first filtering step includes retrieving, as output from the second model, a corresponding second plurality of interaction features for a complex formed between the respective candidate molecule and an off-target macromolecule or off-target macromolecule complex, other than the target macromolecule or target macromolecule complex. In some embodiments, at least the first plurality of interaction features or the second plurality of interaction features are used to obtain a corresponding second score for the interaction between the respective candidate molecule and the target macromolecule or the target macromolecule complex. In some embodiments, one or more candidate molecules are removed from the plurality of candidate molecules based on an evaluation of the corresponding second score for each respective candidate molecule in the plurality of candidate molecules, where the first model comprises a first plurality of at least 1000 parameters and the second model comprises a second plurality of at least 1000 parameters.

In some embodiments, a second filtering step is performed for the plurality of candidate molecules, including (i) for each respective candidate molecule in the plurality of candidate molecules: determining a respective third plurality of interaction features or a respective fourth plurality of interaction features for the respective candidate molecule. In some implementations, each respective interaction feature in the third plurality of interaction features is associated with a binding affinity between the respective candidate molecule and the target macromolecule or the target macromolecule complex, and each respective interaction feature in the fourth plurality of interaction features is associated with a binding specificity between the respective candidate molecule and the target macromolecule or the target macromolecule complex. In some embodiments, the second filtering step further includes (ii) removing one or more candidate molecules from the plurality of candidate molecules based at least on a count of interaction features, in one or both of the respective third plurality of interaction features and the respective fourth plurality of interaction features, for each respective candidate molecule in the plurality of candidate molecules.

In some embodiments, the method further includes determining, for each respective candidate molecule in the plurality of candidate molecules, a corresponding prediction of interaction between the respective candidate molecule and the target macromolecule or the target macromolecule complex, where the prediction is obtained using at least the third plurality of interaction features or the fourth plurality of interaction features corresponding to the respective candidate molecule.

Another aspect of the present disclosure includes a system for characterizing an interaction between a candidate molecule and a target macromolecule or a target macromolecule complex, including a memory; one or more processors; and one or more modules stored in the memory and configured for execution by the one or more processors, the one or more modules including instructions for performing any of the methods disclosed above.

Another aspect of the present disclosure includes a non-transitory computer readable storage medium for characterizing an interaction between a candidate molecule and a target macromolecule or a target macromolecule complex, the non-transitory computer readable storage medium storing one or more programs for execution by one or more processors of a computer system, the one or more computer programs including instructions for performing any of the methods disclosed above.

Yet another aspect of the present disclosure provides a method for identifying a candidate molecule having a target activity with a target macromolecule or a target macromolecule complex, the method including obtaining a plurality of molecular reactions and a plurality of at least 1×10⁶ molecular components. A procedure is performed including i) obtaining, for each respective molecular component in the set of molecular components, a respective transformation of the respective molecular component that represents a corresponding one or more molecular reactions in the plurality of molecular reactions, thereby generating a plurality of molecular intermediates. The procedure further includes ii) removing, from the plurality of molecular intermediates, one or more respective molecular intermediates based on a respective first score for a binding interaction between each respective molecular intermediate in the plurality of molecular intermediates and the target macromolecule or the target macromolecule complex, where, for each respective molecular intermediate in the plurality of molecular intermediates, the respective first score is obtained using at least a corresponding first plurality of interaction features for a complex formed between the respective molecular intermediate and the target macromolecule or target macromolecule complex. The procedure further includes iii) assigning, after the removing, the plurality of molecular intermediates to the plurality of molecular components. The obtaining i), removing ii), and assigning iii) are repeated until a respective second score for the binding interaction between each respective molecular intermediate in the plurality of molecular intermediates and the target macromolecule or target macromolecule complex satisfies a threshold exit criterion, where, for each respective molecular intermediate in the plurality of molecular intermediates, the respective second score is obtained using at least a corresponding second plurality of interaction features for a complex formed between the respective molecular intermediate and the target macromolecule or target macromolecule complex. The procedure further includes v) generating a collection of candidate molecules using the plurality of molecular intermediates, where the plurality of candidate molecules comprises at least 1×10⁶ candidate molecules. The method further includes determining, for each respective candidate molecule in the plurality of candidate molecules, a corresponding prediction of the target activity between the respective candidate molecule and the target macromolecule or target macromolecule complex.

Still another aspect of the present disclosure provides a method for characterizing an interaction between a candidate molecule and a target macromolecule or a target macromolecule complex. In some embodiments, the method is performed at a computer system comprising one or more processing cores and a memory. In some embodiments, the method includes selecting a plurality of candidate molecules from a collection of candidate molecules based on a respective first score for the interaction between each respective candidate molecule in the collection of candidate molecules and the target macromolecule or the target macromolecule complex, where the plurality of candidate molecules comprises at least 1×10⁶ candidate molecules.

In some embodiments, a first filtering step for the plurality of candidate molecules is performed, comprising, for each respective candidate molecule in the plurality of candidate molecules, inputting a representation of a chemical structure of the respective candidate molecule into a first model comprising a first plurality of parameters, where the first model applies the first plurality of parameters to the representation of the chemical structure through at least 10,000 instructions to generate, as output from the first model, a corresponding first plurality of interaction features for a complex formed between the respective candidate molecule and the target macromolecule or the target macromolecule complex, or inputting the representation of the chemical structure of the respective candidate molecule into a second model comprising a second plurality of parameters, where the second model applies the second plurality of parameters to the representation of the molecular structure through at least 10,000 instructions to generate, as output from the second model, a corresponding second plurality of interaction features for a complex formed between the respective candidate molecule and an off-target macromolecule or off-target macromolecule complex, other than the target macromolecule or target macromolecule complex.

In some embodiments, at least the first plurality of interaction features or the second plurality of interaction features is used to obtain a corresponding second score for the interaction between the respective candidate molecule and the target macromolecule or the target macromolecule complex. In some embodiments, one or more candidate molecules are removed from the plurality of candidate molecules based on an evaluation of the corresponding second score for each respective candidate molecule in the plurality of candidate molecules. In some embodiments, the first plurality of parameters comprises 1000 parameters and the second plurality of parameters comprises at least 1000 parameters.

In some embodiments, a second filtering step for the plurality of candidate molecules is performed comprising (i) for each respective candidate molecule in the plurality of candidate molecules, determining a respective third plurality of interaction features or a respective fourth plurality of interaction features for the respective candidate molecule. In some embodiments, each respective interaction feature in the third plurality of interaction features is associated with a binding affinity between the respective candidate molecule and the target macromolecule or the target macromolecule complex, and each respective interaction feature in the fourth plurality of interaction features is associated with a binding specificity between the respective candidate molecule and the target macromolecule or the target macromolecule complex. In some embodiments, the second filtering step further includes (ii) removing one or more candidate molecules from the plurality of candidate molecules based at least on a count of interaction features, in one or both of the respective third plurality of interaction features and the respective fourth plurality of interaction features, for each respective candidate molecule in the plurality of candidate molecules.

In some embodiments, for each respective candidate molecule in the plurality of candidate molecules, a corresponding prediction of interaction between the respective candidate molecule and the target macromolecule or the target macromolecule complex is determined, where the prediction is obtained using at least the third plurality of interaction features or the fourth plurality of interaction features corresponding to the respective candidate molecule.

In some embodiments, the representation of the molecular structure is a two-dimensional molecular graph comprising a plurality of nodes and a plurality of edges, where each respective node in the plurality of nodes represents a corresponding atom in the respective candidate molecule and each respective edge in the plurality of edges represents a covalent bond between a pair of atoms in the respective candidate molecule represented by a corresponding pair of nodes in the plurality of nodes.

### BRIEF DESCRIPTION OF THE DRAWINGS

In the drawings, embodiments of the systems and methods of the present disclosure are illustrated by way of example. It is to be expressly understood that the description and drawings are only for the purpose of illustration and as an aid to understanding, and are not intended as a definition of the limits of the systems and methods of the present disclosure.

FIGS. 1A, 1B, 1C, and 1D collectively illustrate a computer system in accordance with some embodiments of the present disclosure.

FIGS. 2A, 2B, 2C, 2D, 2E, 2F, 2G, 2H, 2I, and 2J collectively illustrate methods for characterizing an interaction between a candidate molecule and a target macromolecule or a target macromolecule complex, in which optional steps are indicated by dashed lines, in accordance with some embodiments of the present disclosure.

FIG. 3 is a schematic view of an example workflow for characterizing an interaction between a candidate molecule and a target macromolecule or a target macromolecule complex, in which optional steps are indicated by dashed lines, in accordance with some embodiments of the present disclosure.

FIGS. 4A, 4B, 4C, and 4D collectively provide a schematic view of an example workflow for characterizing an interaction between a candidate molecule and a target macromolecule or a target macromolecule complex, in accordance with some embodiments of the present disclosure.

FIGS. 5A, 5B, and 5C provide example data structures for use in an example workflow for characterizing an interaction between a candidate molecule and a target macromolecule or a target macromolecule complex, in accordance with some embodiments of the present disclosure.

FIGS. 6A, 6B, 6C, and 6D provide example data structures for use in an example workflow for characterizing an interaction between a candidate molecule and a target macromolecule or a target macromolecule complex, in accordance with some embodiments of the present disclosure.

FIGS. 7A, 7B, 7C, 7D, and 7E provide example data structures for use in an example workflow for characterizing an interaction between a candidate molecule and a target macromolecule or a target macromolecule complex, in accordance with some embodiments of the present disclosure.

FIGS. 8A, 8B, and 8C collectively provide a schematic view of an example workflow for characterizing an interaction between a candidate molecule and a target macromolecule or a target macromolecule complex, in accordance with some embodiments of the present disclosure.

FIG. 9 illustrates an example representation of a transformed interaction feature vector, in which values for interaction features are binarized, in accordance with some embodiments of the present disclosure.

FIGS. 10A and 10B illustrate a comparison of interaction feature scores obtained for candidate molecules generated by methods provided herein relative to scores obtained for test molecules from a commercial library, in accordance with some embodiments of the present disclosure.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Drug discovery efforts often suffer from significant bottlenecks, including the ability to identify hit compounds and validate any such identified hit compounds as lead compounds for eventually synthesis and testing. These difficulties can be attributed, at least in part, to the massive size of custom molecule libraries that are searched in these early stages, which can reach up to 10¹² candidate molecules. Conventional methods, including traditional screening, fragment-based screening, and various machine learning and artificial intelligence pipelines, require laborious hit identification and/or hit-to-lead steps that increase the overall time, cost, and resource expenditure of drug discovery.

Advantageously, the systems and methods disclosed herein allow for rational design of molecules that meet stringent binding, selectivity, and pharmacological requirements using machine learning approaches that convert predictions into data by synthesizing and assaying candidate molecules at scale. In particular, the systems and methods disclosed herein provide a unique chemistry platform that can be used to identify lead-like candidate molecules optimized for selectivity and drug-likeness such as toxicity and ADME properties in ultra-large custom libraries for undruggable targets or complex environments such as brain penetration. For instance, as described in Example 1 below, the disclosed systems and methods improve lead compound identification at a higher hit rate, potency, selectivity, and cost-efficiency compared to conventional methods.

Accordingly, the present disclosure provides systems and methods for characterizing an interaction between a candidate molecule and a target macromolecule or a target macromolecule complex. In some embodiments, candidate molecules are selected from a collection of candidate molecules based on first scores for the interaction between the molecules and a target. A first filtering step is performed by inputting, for each candidate molecule, a molecular graph of the candidate molecule into a first model to retrieve a first plurality of on-target interaction features. Alternatively or additionally, the molecular graph of the candidate molecule is inputted into a second model to retrieve a second plurality of off-target interaction features. Second interaction scores are obtained using the first plurality of interaction features and/or the second plurality of interaction features. Molecules are removed from the plurality of candidate molecules based on an evaluation of the second score. A second filtering step is performed by determining, for each candidate molecule, a third plurality of interaction features associated with binding affinity between the candidate molecule and the target and/or a fourth plurality of interaction features associated with binding specificity between the candidate molecule and the target. Molecules are removed from the plurality of candidate molecules based at least on a count of interaction features in one or both of the third plurality of interaction features and the fourth plurality of interaction features. For each candidate molecule, a prediction of interaction between the molecule and the target is determined using at least the third plurality of interaction features and/or the fourth plurality of interaction features.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

### Definitions.

The terminology used in the present disclosure is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used interchangeably herein, the terms “macromolecule,” “macromolecule complex,” or “polymer” refer to a biological object that is capable of interacting with a molecule. In some embodiments, a macromolecule is a protein, a polypeptide, a polynucleic acid, a polyribonucleic acid, a polysaccharide, or an assembly of any combination thereof. In some embodiments, a macromolecule is a large molecule composed of repeating residues. In some embodiments, the macromolecule is a natural material. In some embodiments, the macromolecule is a synthetic material. In some embodiments, the macromolecule is an elastomer, shellac, amber, natural or synthetic rubber, cellulose, Bakelite, nylon, polystyrene, polyethylene, polypropylene, polyacrylonitrile, polyethylene glycol, or a polysaccharide.

In some embodiments, the macromolecule is a heteropolymer (copolymer). A copolymer is a polymer derived from two (or more) monomeric species, as opposed to a homopolymer where only one monomer is used. Copolymerization refers to methods used to chemically synthesize a copolymer. Examples of copolymers include, but are not limited to, ABS plastic, SBR, nitrile rubber, styrene-acrylonitrile, styrene-isoprene-styrene (SIS) and ethylene-vinyl acetate. Since a copolymer comprises at least two types of constituent units (also structural units, or particles), copolymers can be classified based on how these units are arranged along the chain. These include alternating copolymers with regular alternating A and B units. See, for example, Jenkins, 1996, “Glossary of Basic Terms in Polymer Science,” Pure Appl. Chem. 68 (12): 2287-2311, which is hereby incorporated herein by reference in its entirety. Additional examples of copolymers are periodic copolymers with A and B units arranged in a repeating sequence (e.g., (A-B-A-B-B-A-A-A-A-B-B-B)₁). Additional examples of copolymers are statistical copolymers in which the sequence of monomer residues in the copolymer follows a statistical rule. See, for example, Painter, 1997, Fundamentals of Polymer Science, CRC Press, 1997, p 14, which is hereby incorporated by reference herein in its entirety. Still other examples of copolymers that may be evaluated using the disclosed systems and methods are block copolymers comprising two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively.

In some embodiments, the macromolecule is a plurality of polymers (e.g., 2 or more, 3, or more, 10 or more, 100 or more, 1000 or more, or 5000 or more polymers), where the respective polymers in the plurality of polymers do not all have the same molecular weight. In some such embodiments, the polymers in the plurality of polymers share at least 50 percent, at least 60 percent, at least 70 percent, at least 80 percent, or at least 90 percent sequence identity and fall into a weight range with a corresponding distribution of chain lengths. In some embodiments, the macromolecule is a branched polymer molecule comprising a main chain with one or more substituent side chains or branches. Types of branched polymers include, but are not limited to, star polymers, comb polymers, brush polymers, dendronized polymers, ladders, and dendrimers. See, for example, Rubinstein et al., 2003, Polymer physics, Oxford; New York: Oxford University Press. p. 6, which is hereby incorporated by reference herein in its entirety.

In some embodiments, the macromolecule is a polypeptide. As used herein, the term “polypeptide” means two or more amino acids or residues linked by a peptide bond. The terms “polypeptide” and “protein” are used interchangeably herein and include oligopeptides and peptides. An “amino acid,” “residue” or “peptide” refers to any of the twenty standard structural units of proteins as known in the art, which include imino acids, such as proline and hydroxyproline. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline and homocysteine, as nonlimiting examples, are all considered amino acids. Other variants or analogs of the amino acids are known in the art. Thus, a polypeptide may include synthetic peptidomimetic structures such as peptoids. See Simon et al., 1992, Proceedings of the National Academy of Sciences USA, 89, 9367, which is hereby incorporated by reference herein in its entirety. See also Chin et al., 2003, Science 301, 964; and Chin et al., 2003, Chemistry & Biology 10, 511, each of which is incorporated by reference herein in its entirety.

In some embodiments, the macromolecule includes any number of posttranslational modifications. Thus, in some embodiments, a macromolecule includes those polymers that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphotases and kinases). Other types of posttranslational modifications are known in the art and are within the scope of the macromolecules or macromolecule complexes of the present disclosure.

In some embodiments, the macromolecule is a surfactant. Surfactants are compounds that lower the surface tension of a liquid, the interfacial tension between two liquids, or that between a liquid and a solid. Surfactants may act as detergents, wetting agents, emulsifiers, foaming agents, and dispersants. Surfactants are usually organic compounds that are amphiphilic, meaning they contain both hydrophobic groups (their tails) and hydrophilic groups (their heads). Therefore, a surfactant molecule contains both a water insoluble (or oil soluble) component and a water-soluble component. Surfactant molecules will diffuse in water and adsorb at interfaces between air and water or at the interface between oil and water, in the case where water is mixed with oil. The insoluble hydrophobic group may extend out of the bulk water phase, into the air or into the oil phase, while the water-soluble head group remains in the water phase. This alignment of surfactant molecules at the surface modifies the surface properties of water at the water/air or water/oil interface. Examples of ionic surfactants include ionic surfactants such as anionic, cationic, or zwitterionic (ampoteric) surfactants.

In some embodiments, the macromolecule is a reverse micelle or liposome. In some embodiments, the target macromolecule is a fullerene. A fullerene is any molecule composed entirely of carbon, in the form of a hollow sphere, ellipsoid or tube. Spherical fullerenes are also called buckyballs, and they resemble the balls used in association football. Cylindrical ones are called carbon nanotubes or buckytubes. Fullerenes are similar in structure to graphite, which is composed of stacked graphene sheets of linked hexagonal rings; but they may also contain pentagonal (or sometimes heptagonal) rings.

In some embodiments, the macromolecule includes two different types of polymers, such as a nucleic acid bound to a polypeptide. In some embodiments, the target macromolecule includes two polypeptides bound to each other. In some embodiments, the target macromolecule includes one or more metal ions (e.g., a metalloproteinase with one or more zinc atoms).

As used herein, the term “target” refers to an object of interest, such as a macromolecule, macromolecule complex, or polymer that is of interest as a primary binding target for a candidate molecule. As used herein, the term “off-target” refers to an object that is not the primary binding target, such as a macromolecule, macromolecule complex, or polymer that exhibits off-target binding with a candidate molecule.

As used interchangeably herein, the terms “pose” or “conformation” refer to a pose of a molecule when complexed to a target or off-target object. In some embodiments, a pose refers to the complex formed between a target or off-target object and any suitable molecule capable of complexing to the target, including but not limited to a candidate molecule, a ligand, a reference molecule, a training molecule, a molecular component, and/or a molecular intermediate.

In some embodiments, a pose is determined one or more docking programs. In some embodiments, one docking program is used to determine some of the poses for a molecule and another docking program is used to determine other poses for the molecule.

In some embodiments, one or more poses are determined using AutoDock Vina. See, Trott and Olson, “AutoDock Vina: improving the speed and accuracy of docking with a new scoring function, efficient optimization and multithreading,” Journal of Computational Chemistry 31 (2010) 455-461. In some embodiments, one or more poses are determined using Quick Vina 2 (Alhossary et al., 2015, “Fast, accurate, and reliable molecular docking with QuickVina,” Bioinformatics 31:13, pp. 2214-2216), VinaLC (Zhang et al., 2013, “Message Passing Interface and Multithreading Hybrid for Parallel Molecular Docking of Large Databases on Petascale High Performance Computing Machines,” J. Comput. Chem. DOI: 10.1002/jcc.23214), Smina (Koes et al, 2013, “Lessons learned in empirical scoring with smina from the CSAR 2011 benchmarking exercise,” Journal of chemical information and modeling 53:8, pp. 1893-1904), or CUina (Morrison et al., “Efficient GPU Implementation of AutoDock Vina,” COMP poster 3432389).

In some embodiments, one or more ensembled poses are determined using an ensembled docking algorithm such as disclosed in Stafford et al., 2022, “AtomNet PoseRanker: Enriching Ligand Pose Quality for Dynamic Proteins in Virtual High-Throughput Screens,” Journal of Chemical Information and Modeling 62, pp. 1178-1189, which is hereby incorporated by reference. In some such embodiments the ensemble consists of between 3 and 64, between 4 and 128, between 5 and 32, more than 5, or between 8 and 25 structurally similar poses.

In some embodiments, the molecule is docked to a target or off-target object by either random pose generation techniques or by biased pose generation. In some embodiments, the molecule is docked to a target or off-target object by Markov chain Monte Carlo sampling. In some embodiments, such sampling allows the full flexibility of the molecule in the docking calculations and a scoring function that is the sum of the interaction energy between the molecule and the target or off-target object as well as the conformational energy of the molecule. See, for example, Liu and Wang, 1999, “MCDOCK: A Monte Carlo simulation approach to the molecular docking problem,” Journal of Computer-Aided Molecular Design 13, 435-451, which is hereby incorporated by reference.

In some embodiments, algorithms such as DOCK (Shoichet, Bodian, and Kuntz, 1992, “Molecular docking using shape descriptors,” Journal of Computational Chemistry 13(3), pp. 380-397; and Knegtel et al., 1997 “Molecular docking to ensembles of protein structures,” Journal of Molecular Biology 266, pp. 424-440, each of which is hereby incorporated by reference) are used to find the one or more poses for a molecule against a target or off-target object. Such algorithms model the target or off-target object and the molecule as rigid bodies. The docked conformation is searched using surface complementary to find poses.

In some embodiments, algorithms such as AutoDOCK (Morris et al., 2009, “AutoDock4 and AutoDockTools4: Automated Docking with Selective Receptor Flexibility,” J. Comput. Chem. 30(16), pp. 2785-2791; Sotriffer et al., 2000, “Automated docking of ligands to antibodies: methods and applications,” Methods: A Companion to Methods in Enzymology 20, pp. 280-291; and “Morris et al., 1998, “Automated Docking Using a Lamarckian Genetic Algorithm and Empirical Binding Free Energy Function,” Journal of Computational Chemistry 19: pp. 1639-1662, each of which is hereby incorporated by reference); FlexX (Rarey et al., 1996, “A Fast Flexible Docking Method Using an Incremental Construction Algorithm,” Journal of Molecular Biology 261, pp. 470-489, which is hereby incorporated by reference); GOLD (Jones et al., 1997, “Development and Validation of a Genetic Algorithm for flexible Docking,” Journal Molecular Biology 267, pp. 727-748, which is hereby incorporated by reference) are used to find one or more poses.

In some embodiments, molecular dynamics is performed on a target or off-target object (or a portion thereof such as the active site of the target or off-target object) and a molecule to identify one or more poses. During the molecular dynamics run, the atoms of the target or off-target object and the molecule are allowed to interact for a fixed period of time, giving a view of the dynamical evolution of the system. The trajectory of atoms in the target or off-target object and the molecule are determined by numerically solving Newton's equations of motion for a system of interacting particles, where forces between the particles and their potential energies are calculated using interatomic potentials or molecular mechanics force fields. See Alder and Wainwright, 1959, “Studies in Molecular Dynamics. I. General Method,” J. Chem. Phys. 31 (2): 459; and Bibcode, 1959, J. Ch. Ph. 31, 459A, doi:10.1063/1.1730376, each of which is hereby incorporated by reference. Thus, in this way, the molecular dynamics run produces a trajectory of the target or off-target object and the respective molecule over time. This trajectory comprises the trajectory of the atoms in the target or off-target object and the molecule. In some embodiments, a subset of the plurality of different poses is obtained by taking snapshots of this trajectory over a period of time. In some embodiments, poses are obtained from snapshots of several different trajectories, where each trajectory comprises a different molecular dynamics run of the target or off-target object interacting with the molecule. In some embodiments, prior to a molecular dynamics run, the molecule is first docked into an active site of the target or off-target object using a docking technique.

As used herein, the term “parameter” refers to any coefficient or, similarly, any value of an internal or external element (e.g., a weight and/or a hyperparameter) in an algorithm, model, regressor, and/or classifier that affects (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that is used to control, modify, tailor, and/or adjust the behavior, learning and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable an algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods, as described elsewhere herein).

In some embodiments, an algorithm, model, regressor, and/or classifier of the present disclosure comprises a plurality of parameters. In some embodiments the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

As used herein, the term “instruction” refers to an order given to a computer processor by a computer program. On a digital computer, in some embodiments, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

Example Systems for Characterizing Interactions between Molecules and Targets

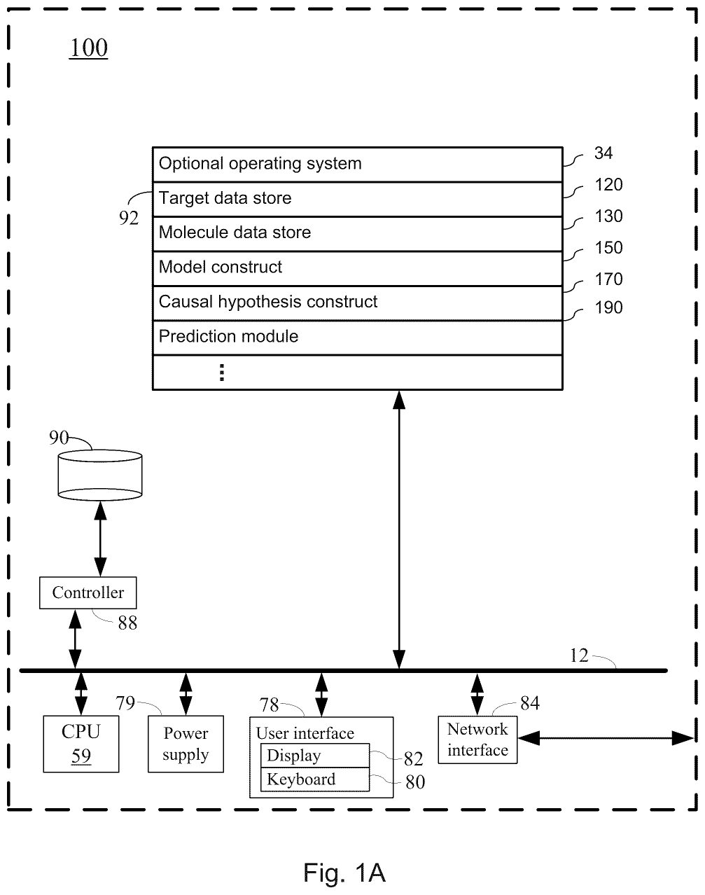

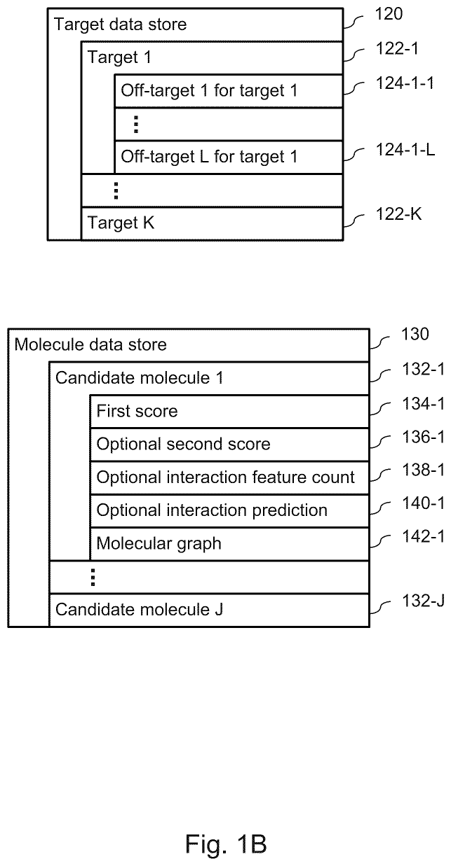

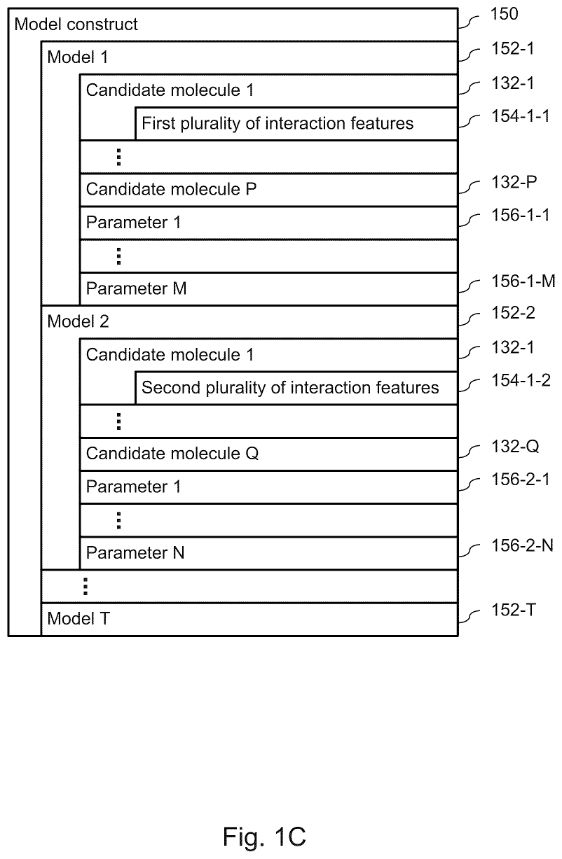

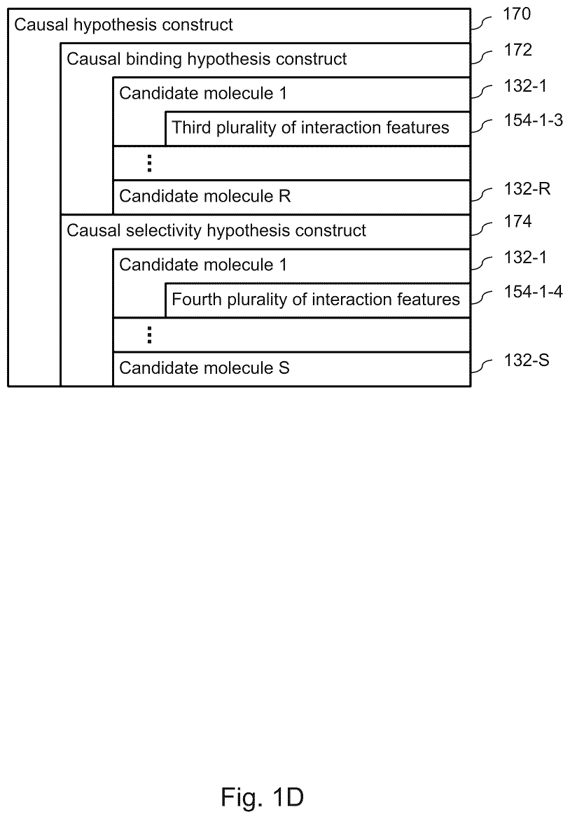

FIGS. 1A-D collectively illustrate a computer system 100 for characterizing an interaction between a candidate molecule and a target macromolecule or target macromolecule complex. For instance, it can be used as a prediction system to generate accurate predictions regarding the binding affinity, binding specificity, activity (e.g., ADME properties), and/or molecular dynamics of one or more candidate molecules with a target macromolecule or target macromolecule complex.

Referring to FIGS. 1A-D, in typical embodiments, computer system 100 comprises one or more computers. For purposes of illustration in FIGS. 1A-D, the computer system 100 is represented as a single computer that includes all of the functionality of the disclosed computer system 100. However, the present disclosure is not so limited. The functionality of the computer system 100 can be spread across any number of networked computers and/or reside on each of several networked computers and/or virtual machines. One of skill in the art will appreciate that a wide array of different computer topologies are possible for the computer system 100 and all such topologies are within the scope of the present disclosure.

Turning to FIGS. 1A-D with the foregoing in mind, the computer system 100 comprises one or more processing units (CPUs) 59, a network or other communications interface 84, a user interface 78 (e.g., including an optional display 82 and optional keyboard 80 or other form of input device), a memory 92 (e.g., random access memory, persistent memory, or combination thereof), one or more magnetic disk storage and/or persistent devices 90 optionally accessed by one or more controllers 88, one or more communication busses 12 for interconnecting the aforementioned components, and a power supply 79 for powering the aforementioned components. To the extent that components of memory 92 are not persistent, data in memory 92 can be seamlessly shared with non-volatile memory 90 or portions of memory 92 that are non-volatile/persistent using known computing techniques such as caching. Memory 92 and/or memory 90 can include mass storage that is remotely located with respect to the central processing unit(s) 59. In other words, some data stored in memory 92 and/or memory 90 may in fact be hosted on computers that are external to computer system 100 but that can be electronically accessed by the computer system 100 over an Internet, intranet, or other form of network or electronic cable using network interface 84. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

In some embodiments, the memory 92 of the computer system 100 stores:
an optional operating system 34 that includes procedures for handling various basic system services;
an optional target data store 120 for storing one or more target macromolecules or target macromolecule complexes 122 (e.g., 122-1, . . . 122-K) and, optionally, for each respective target macromolecule or macromolecule complex, a corresponding one or more off-target macromolecules or off-target macromolecule complexes 124 (e.g., 124-1-1, . . . 124-1-L);
a molecule data store 130 for storing a collection of candidate molecules 132 (e.g., 132-1, 132-S, 132-R, 132-Q, 132-P, . . . 132-J), where each respective candidate molecule 132 in the collection of candidate molecules comprises:
   a respective first score 134 (e.g., 134-1) for the interaction between the respective candidate molecule 132 and a target macromolecule or target macromolecule complex 122,
   an optional respective second score 136 (e.g., 136-1) for the interaction between the respective candidate molecule 132 and the target macromolecule or target macromolecule complex 122,
   an optional respective interaction feature count 138 (e.g., 138-1),
   an optional interaction prediction 140 (e.g., 140-1), and
   a molecular graph 142 (e.g., 142-1);
a model construct 150, comprising at least a first model 152 (e.g., 152-1) and/or a second model (e.g., 152-2), and optionally, one or more additional models (e.g., 152-T), where the first model includes a first plurality of parameters 156 (e.g., 156-1, . . . 156-M), the second model includes a second plurality of parameters 156 (e.g., 156-1, . . . 156-N), and, for each respective candidate molecule 132 in a plurality of candidate molecules:
   responsive to inputting the molecular graph 142 for the respective candidate molecule 132, the first model 152-1 outputs a corresponding first plurality of interaction features 154-1 (e.g., 154-1-1) for a complex formed between the respective candidate molecule 132 and the target macromolecule or the target macromolecule complex 122,
   responsive to inputting the molecular graph 142 for the respective candidate molecule 132, the second model 152-2 outputs a corresponding second plurality of interaction features 154-2 (e.g., 154-1-2) for a complex formed between the respective candidate molecule 132 and the target macromolecule or the target macromolecule complex 122, and
   the corresponding second score 136 for the interaction between the respective candidate molecule 132 and the target macromolecule or the target macromolecule complex 122 is obtained using at least the first plurality of interaction features 154-1 and/or the second plurality of interaction features 154-2;
a causal hypothesis construct 170, comprising at least a causal binding hypothesis construct 172 and/or a causal selectivity hypothesis construct 174, where for each respective candidate molecule 132 in the plurality of candidate molecules:
   the causal binding hypothesis construct 172 determines a respective third plurality of interaction features 154-3 (e.g., 154-1-3) for the respective candidate molecule, where each respective interaction feature in the third plurality of interaction features is associated with a binding affinity between the respective candidate molecule 132 and the target macromolecule or the target macromolecule complex 122,
   the causal selectivity hypothesis construct 174 determines a respective fourth plurality of interaction features 154-4 (e.g., 154-1-4) for the respective candidate molecule, where each respective interaction feature in the fourth plurality of interaction features is associated with a binding specificity between the respective candidate molecule 132 and the target macromolecule or the target macromolecule complex 122, and
   the corresponding interaction feature count 138 is determined using at least the third plurality of interaction features 154-3 and/or the fourth plurality of interaction features 154-4; and
a prediction model 190 for determining, for each respective candidate molecule 132 in the plurality of candidate molecules, a corresponding prediction of interaction 140 between the respective candidate molecule 132 and the target macromolecule or the target macromolecule complex 122, where the prediction is obtained using at least the third plurality of interaction features 154-3 and/or the fourth plurality of interaction features 154-4 corresponding to the respective candidate molecule.

In some implementations, any two or more of J, P, Q, R, and S are the same or a different positive integer value. In some implementations, M and N are the same or a different positive integer value.

In some implementations, one or more of the above identified data elements or modules of the computer system 100 are stored in one or more of the previously mentioned memory devices, and correspond to a set of instructions for performing a function described above. The above identified data, modules, or programs (e.g., sets of instructions) need not be implemented as separate software programs, procedures or modules, and thus various subsets of these modules may be combined or otherwise re-arranged in various implementations. In some implementations, the memory 92 and/or 90 (and optionally 52) optionally stores a subset of the modules and data structures identified above. Furthermore, in some embodiments the memory 92 and/or 90 (and optionally 52) stores additional modules and data structures not described above. In some embodiments, the first neural network 72 is replaced with another form of model.

### Example Methods for Characterizing Interactions Between Molecules and Targets

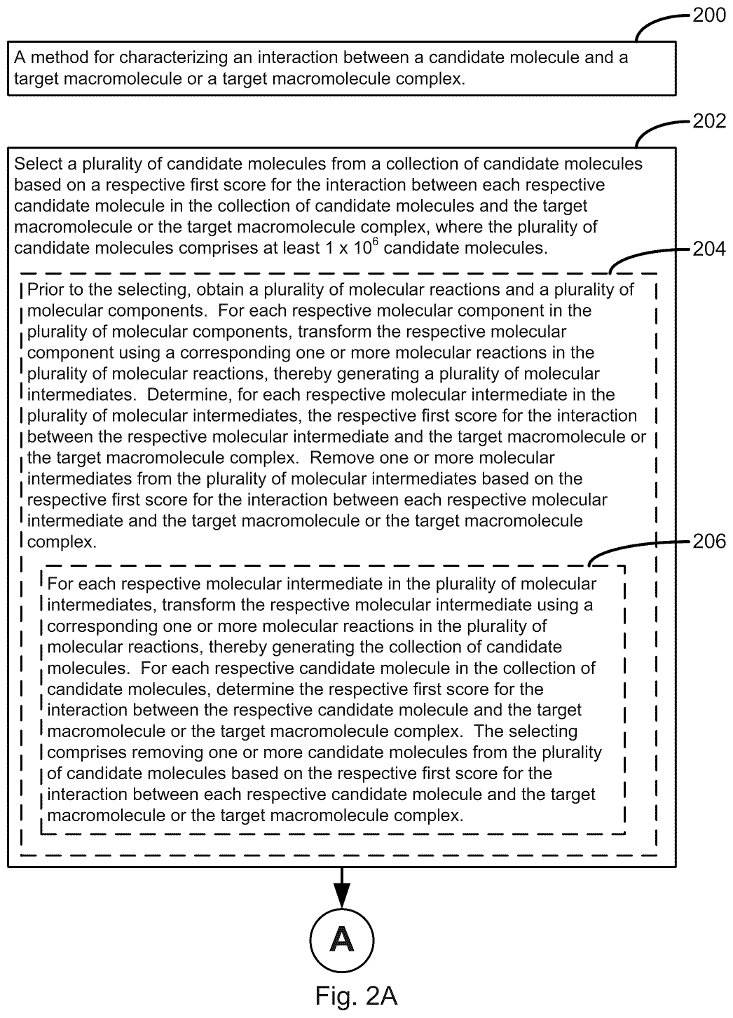

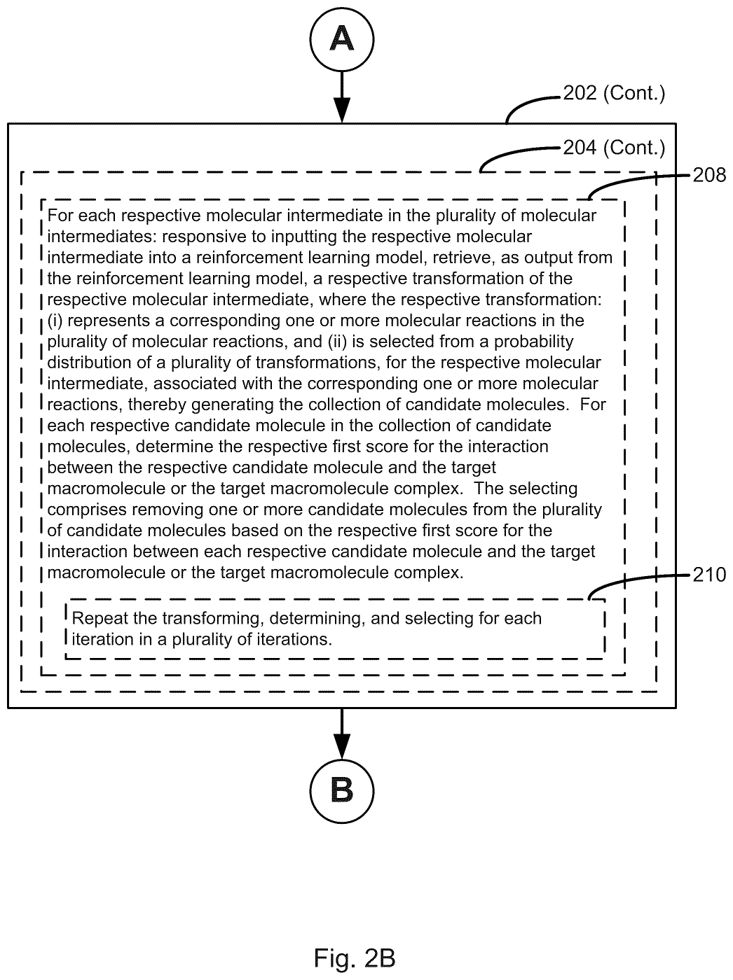

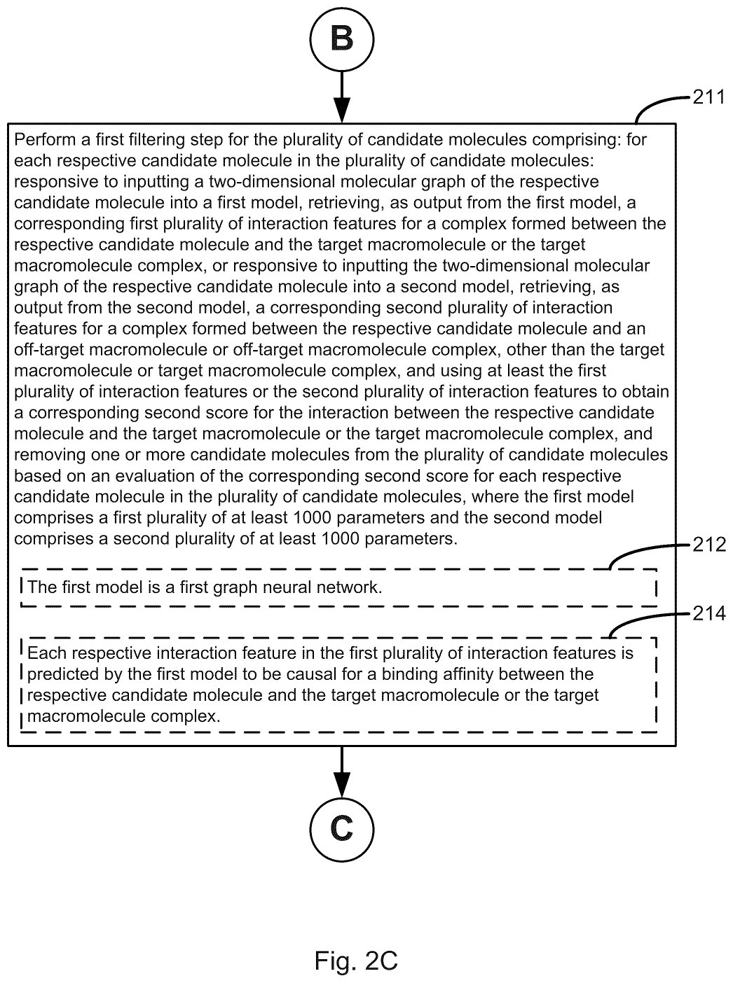

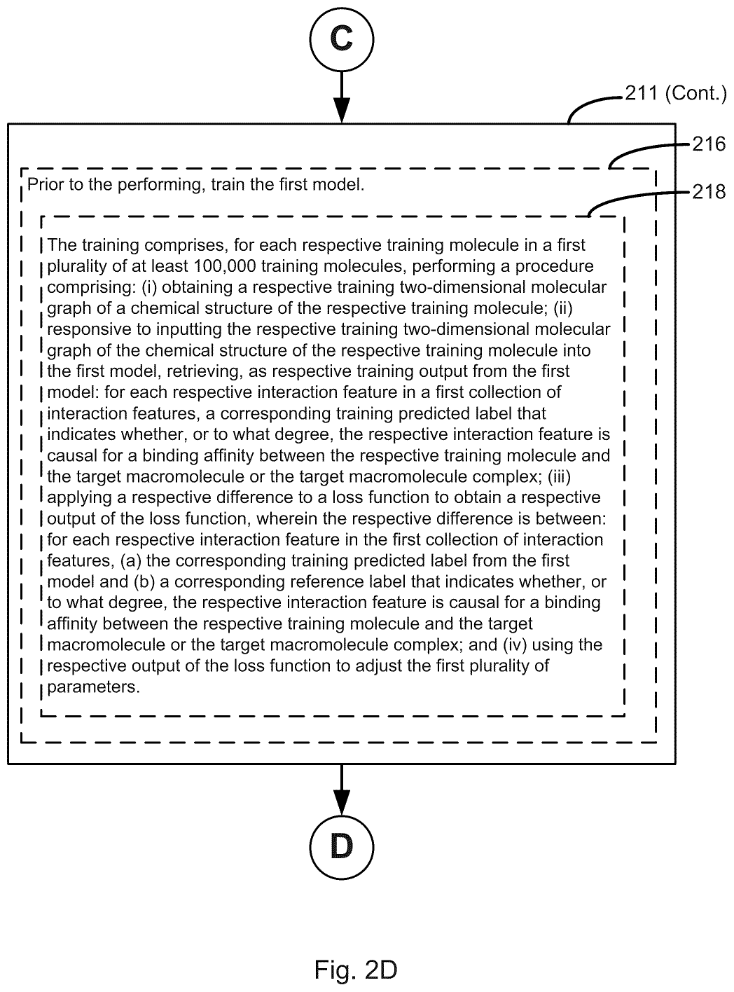

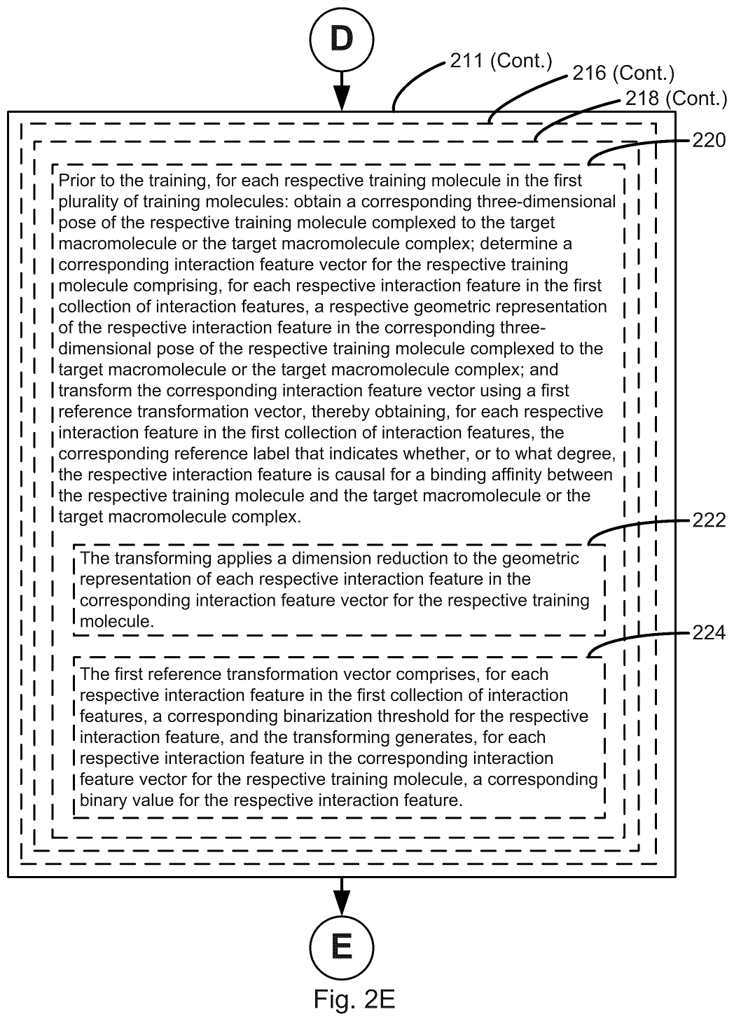

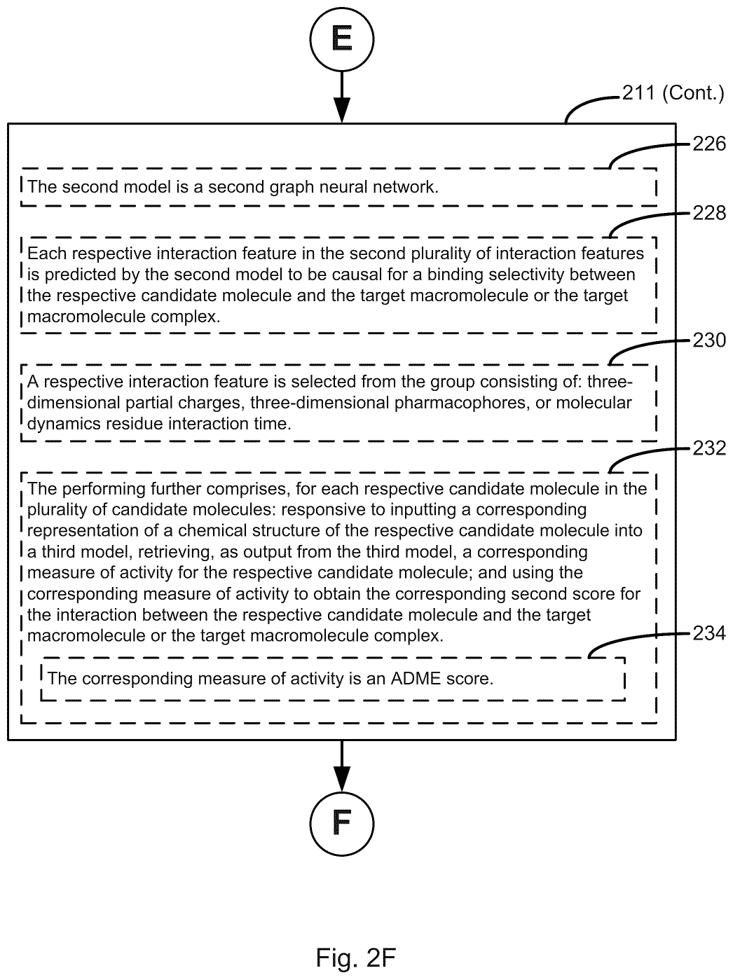

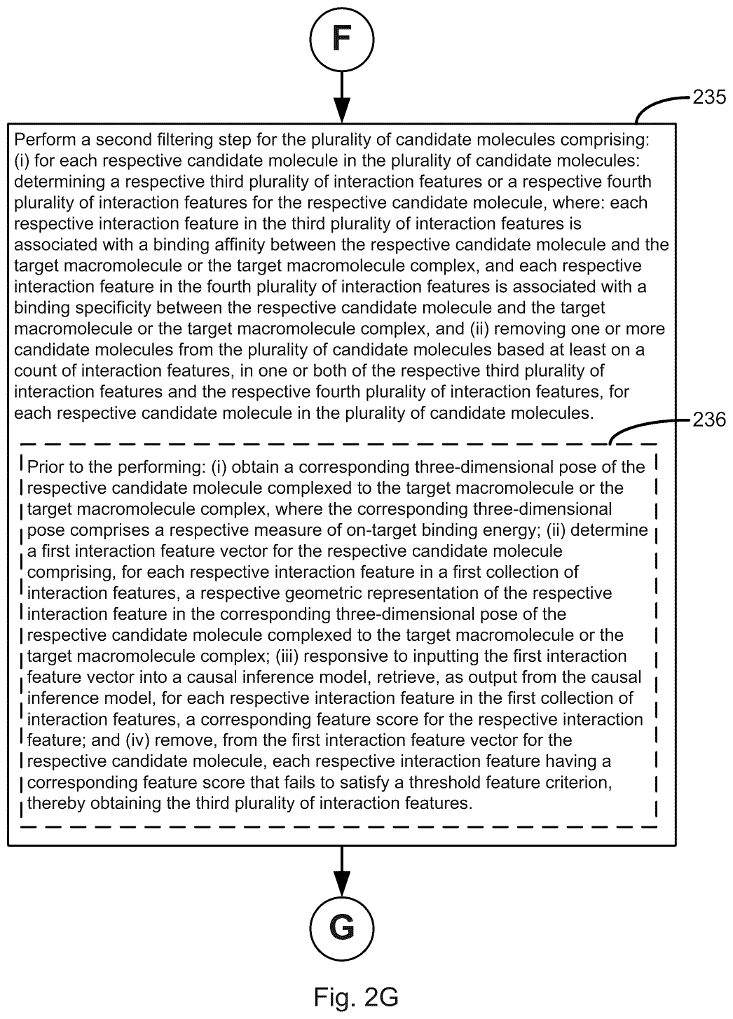

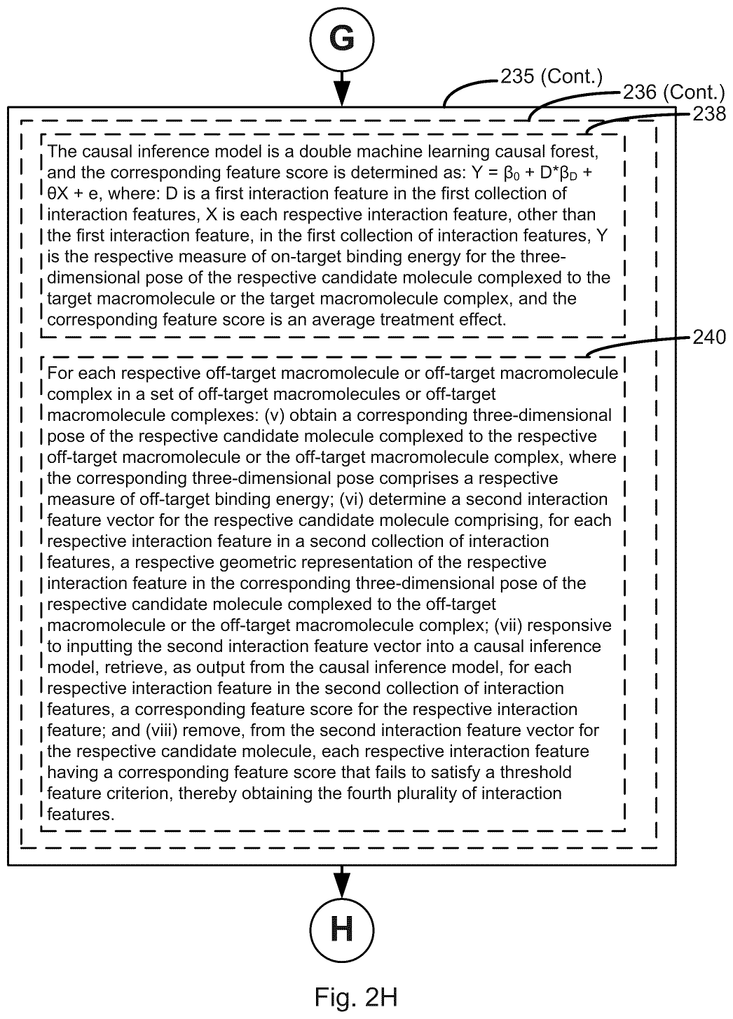

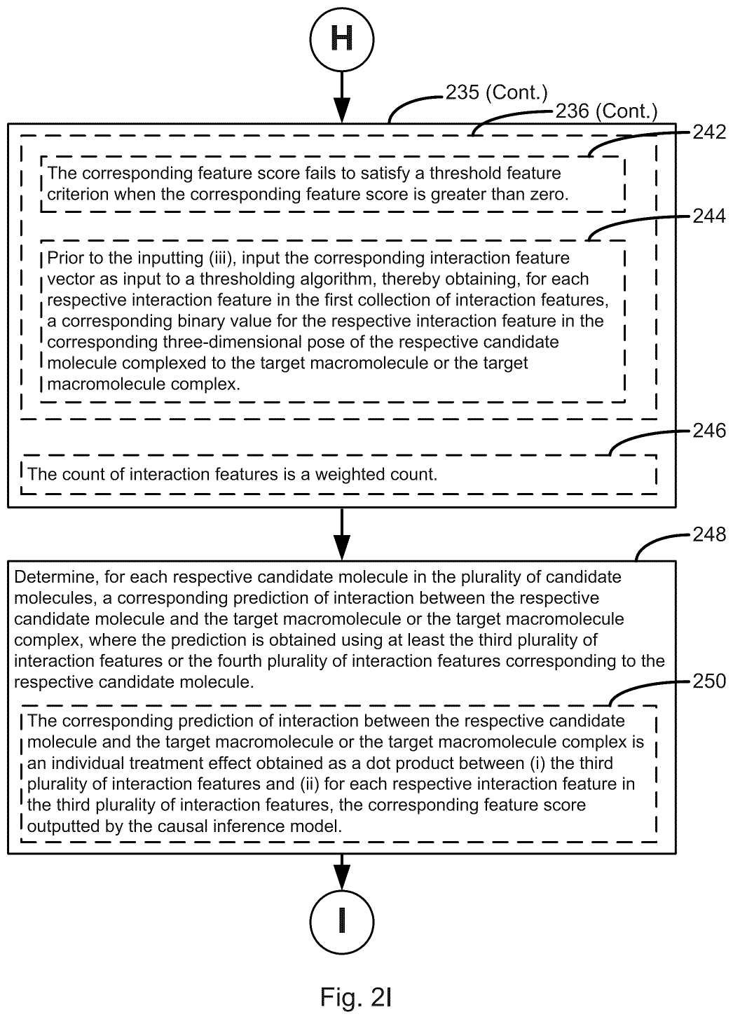

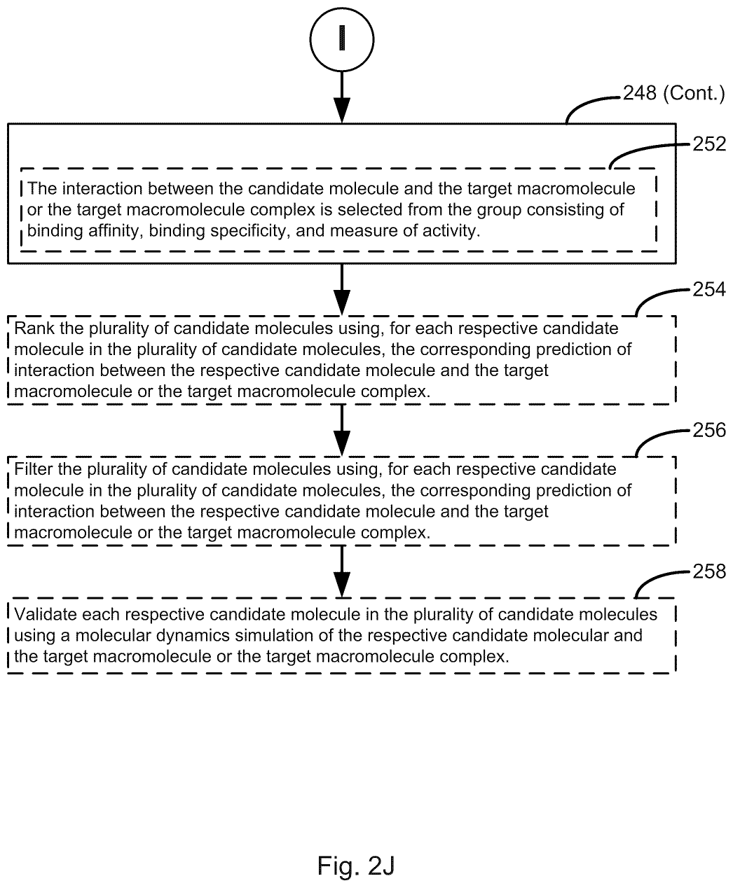

Now that a system for characterizing an interaction between a candidate molecule and a target macromolecule or target macromolecule complex has been disclosed, methods for performing such characterization are detailed with reference to FIGS. 2A-J, 3, 4A-D, 5A-C, 6A-D, 7A-E, and 8A-C and discussed below.

Block 200. Referring to block 200 of FIG. 2A, a method 200 for characterizing an interaction between a candidate molecule 132 and a target macromolecule or a target macromolecule complex 122 is provided. In some embodiments, as discussed above in conjunction with FIGS. 1A-D, the method is performed at a computer system 100 comprising one or more processing cores and a memory.

In some embodiments, the interaction is binding affinity, binding specificity, and/or a measure of activity, such as an ADME property.

### Generating Molecules.

Referring to block 202, the method 200 includes A) selecting a plurality of candidate molecules 132 from a collection of candidate molecules based on a respective first score 134 for the interaction between each respective candidate molecule 132 in the collection of candidate molecules and the target macromolecule or the target macromolecule complex 122.

In some embodiments, the plurality of candidate molecules comprises at least 1×10⁶ candidate molecules. In some embodiments, the first score is an efficiency score.

In some embodiments, the target macromolecule or target macromolecule complex is a protein, a polypeptide, a polynucleic acid, a polyribonucleic acid, a polysaccharide, or an assembly of any combination thereof. In some embodiments, the target macromolecule or target macromolecule complex comprises protein, DNA, RNA, ribosomes, protein-protein complexes, or an assembly or any combination thereof. In some implementations, the target macromolecule or target macromolecule complex comprises any of the embodiments for target objects disclosed herein. See, for example, the section entitled “Definitions: Macromolecules,” above.

In some embodiments, the method 200 is performed for each respective target macromolecule or target macromolecule complex in a plurality of target macromolecules or macromolecule complexes.

In some embodiments, the plurality of targets comprises at least 2, at least 3, at least 4, at least 5, at least 10, at least 20, at least 50, at least 100, at least 500, or at least 1000 targets. In some embodiments, the plurality of targets comprises no more than 5000, no more than 1000, no more than 100, no more than 50, no more than 10, or no more than 5 targets. In some embodiments, the plurality of targets consists of from 2 to 10, from 5 to 50, from 30 to 500, or from 1000 to 5000 targets. In some embodiments, the plurality of targets falls within another range starting no lower than 2 targets and ending no higher than 5000 targets.

In some embodiments, a target macromolecule or target macromolecule complex comprises 50 or more, 100 or more, 150 or more, 200 or more, 300 or more, 400 or more, 500 or more, 600 or more, 700 or more, 800 or more, 900 or more, 1000 or more, or 5000 or more atoms. In some embodiments, a target macromolecule or target macromolecule complex comprises no more than 10,000, no more than 5000, no more than 1000, no more than 500, or no more than 100 atoms. In some embodiments, a target macromolecule or target macromolecule complex consists of from 50 to 100, from 50 to 500, from 100 to 1000, or from 1000 to 10,000 atoms. In some embodiments, a target macromolecule or target macromolecule complex comprises another range of atoms starting no lower than 50 atoms and ending no higher than 10,000 atoms.

In some embodiments, the target macromolecule or macromolecule complex is a polymer comprising 10 or more, 20 or more, 30 or more, 50 or more, 100 or more, or 500 or more residues. In some embodiments, the target macromolecule or macromolecule complex is a polymer comprising no more than 1000, no more than 500, no more than 100, no more than 50, or no more than 20 residues. In some embodiments, the target macromolecule or macromolecule complex is a polymer consisting of from 10 to 100, from 50 to 200, from 100 to 500, or from 500 to 1000 residues. In some embodiments, the target macromolecule or macromolecule complex is a polymer that falls within another range starting no lower than 10 residues and ending no higher than 1000 residues.

In some embodiments, the target macromolecule or macromolecule complex comprises a one or more active sites to which a respective candidate molecule can bind.

In some embodiments, each respective candidate molecule (e.g., in the plurality of candidate molecules and/or in the collection of candidate molecules) is a chemical compound. In some embodiments, each respective candidate molecule (e.g., in the plurality of candidate molecules and/or in the collection of candidate molecules) is a ligand and/or a substrate. In some embodiments, a respective candidate molecule is a large polymer or macromolecule, such as an antibody. In some embodiments, a respective candidate molecule is an organic or inorganic compound.

In some embodiments, a respective candidate molecule satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, the respective candidate molecule satisfies one or more criteria in addition to Lipinski's Rule of Five. For example, in some embodiments, the respective candidate molecule has five or fewer aromatic rings, four or fewer aromatic rings, three or fewer aromatic rings, or two or fewer aromatic rings.

In some embodiments, a respective candidate molecule has a molecular weight of at least 100, at least 500, at least 1000, at least 2000, at least 5000, or at least 10,000 Daltons. In some embodiments, a respective candidate molecule has a molecular weight of no more than 20,000, no more than 10,000, no more than 8000, no more than 6000, no more than 4000, no more than 2000, no more than 1000, or no more than 500 Daltons. In some embodiments, a respective candidate molecule has a molecular weight of from 100 to 500, from 500 to 2000, from 1000 to 8000, or from 5000 to 20,000 Daltons. In some embodiments, a respective candidate molecule has a molecular weight that falls within another range starting no lower than 100 Daltons and ending no higher than 20,000 Daltons. However, some embodiments of the disclosed systems and methods have no limitation on the size of the candidate molecule.

In some embodiments, the method includes selecting a respective plurality of candidate molecules for each respective target in a plurality of target macromolecules or macromolecule complexes. In some such embodiments, each respective target has a corresponding plurality of selected candidate molecules that is the same or different as any other target in a plurality of target macromolecules or macromolecule complexes. For example, where a respective plurality of candidate molecules is a plurality of putative ligands for a binding target, each respective binding target can have a same or different set of ligands that are evaluated for various binding, specificity, or activity properties against the respective binding target.

In some embodiments, for a respective target macromolecule or macromolecule complex, the corresponding plurality of candidate molecules comprises at least 100, at least 500, at least 1000, at least 5000, at least 10,000, at least 100,000, at least 1×10⁶, at least 5×10⁶, at least 1×10⁷, at least 1×10⁸, or at least 5×10⁸ candidate molecules. In some embodiments, for a respective target macromolecule or macromolecule complex, the corresponding plurality of candidate molecules comprises no more than 1×10⁹, no more than 1×10⁸, no more than 1×10⁷, no more than 1×10⁶, no more than 500,000, no more than 100,000, no more than 10,000, or no more than 1000 candidate molecules. In some embodiments, for a respective target macromolecule or macromolecule complex, the corresponding plurality of candidate molecules consists of from 100 to 1000, from 1000 to 100,000, from 10,000 to 5×10⁶, from 1×10⁶ to 1×10⁷, from 1×10⁷ to 1×10⁸, or from 1×10⁸ to 1×10⁸ candidate molecules. In some embodiments, for a respective target macromolecule or macromolecule complex, the corresponding plurality of candidate molecules falls within another range starting no lower than 100 candidate molecules and ending no higher than 1×10⁹ candidate molecules.

In some embodiments, the collection of candidate molecules comprises at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹ candidate molecules. In some embodiments, the collection of candidate molecules comprises no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000 candidate molecules. In some embodiments, the collection of candidate molecules consists of from 1000 to 100,000, from 10,000 to 1×10⁷, from 1×10⁶ to 1×10⁸, from 1×10⁸ to 1×10¹¹, or from 1×10⁹ to 1×10¹² candidate molecules. In some embodiments, the collection of candidate molecules falls within another range starting no lower than 1000 candidate molecules and ending no higher than 1×10¹² candidate molecules.

In some embodiments, a respective candidate molecule is selected from a plurality of molecular components or molecular intermediates, as described below.

Exploration phase.

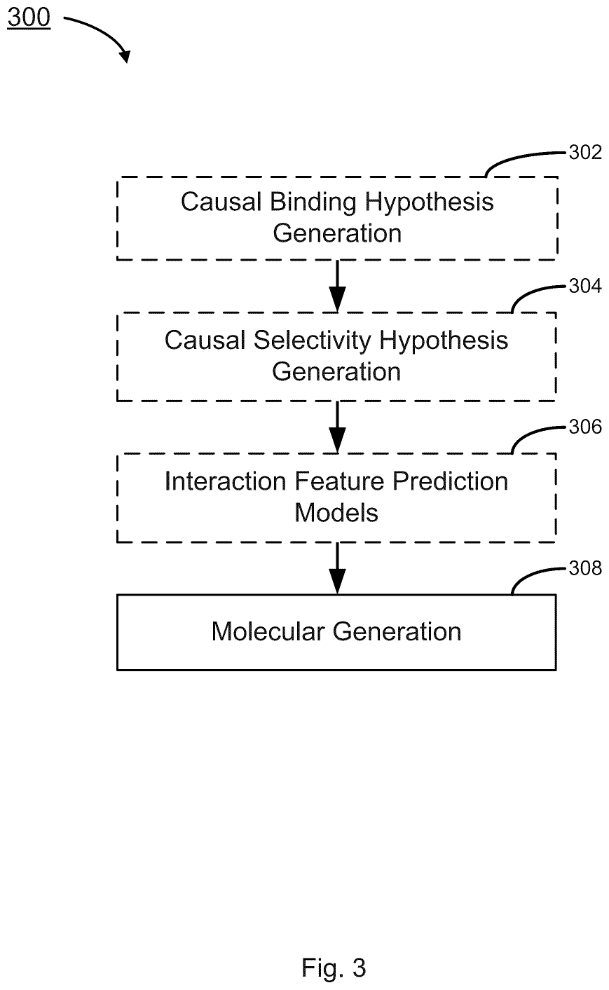

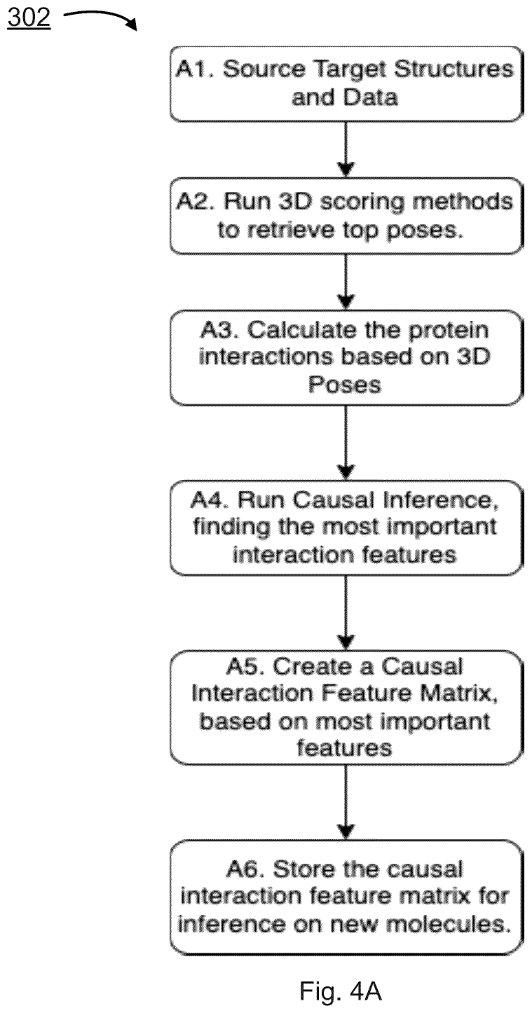

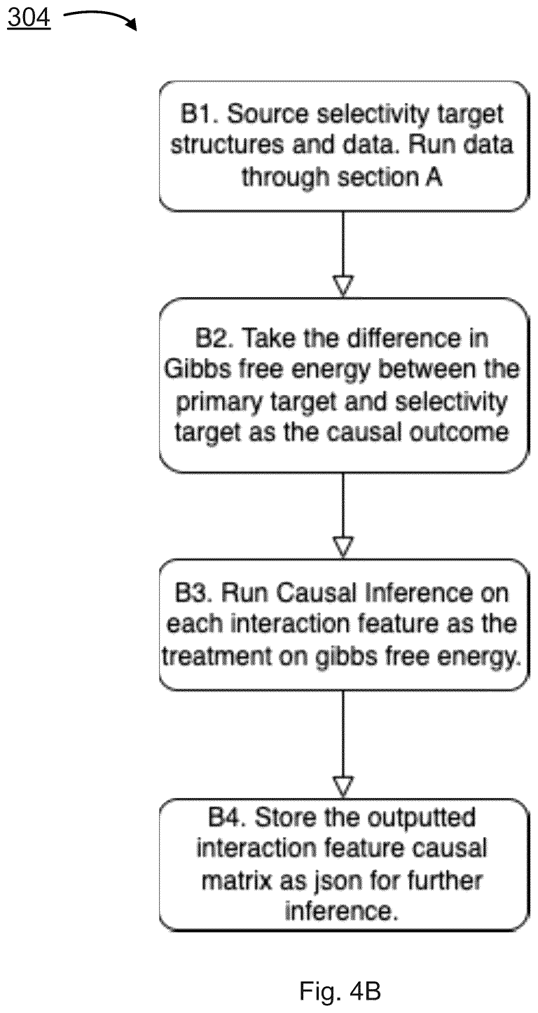

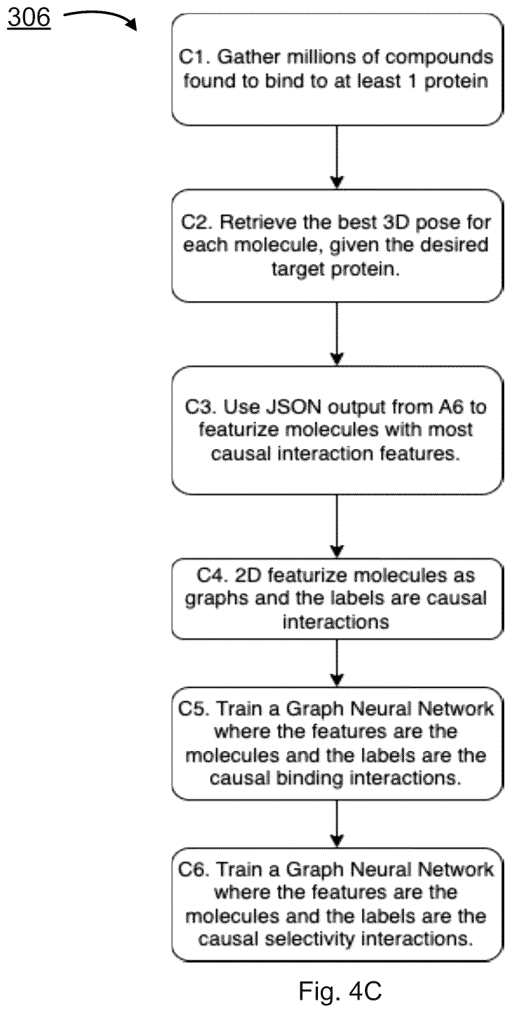

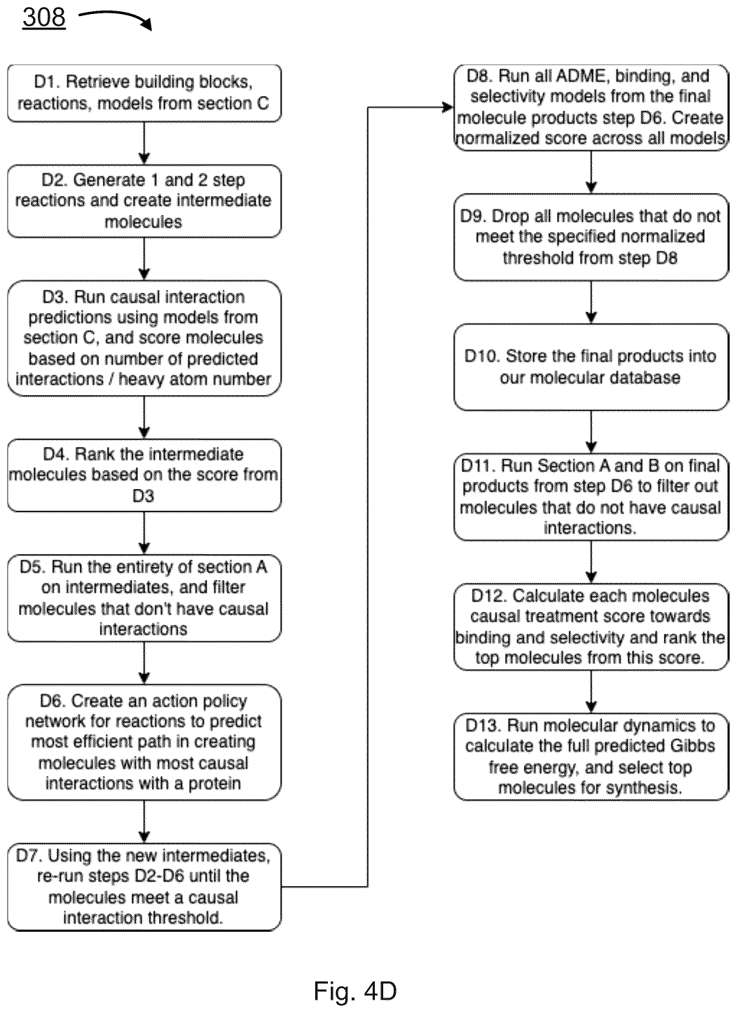

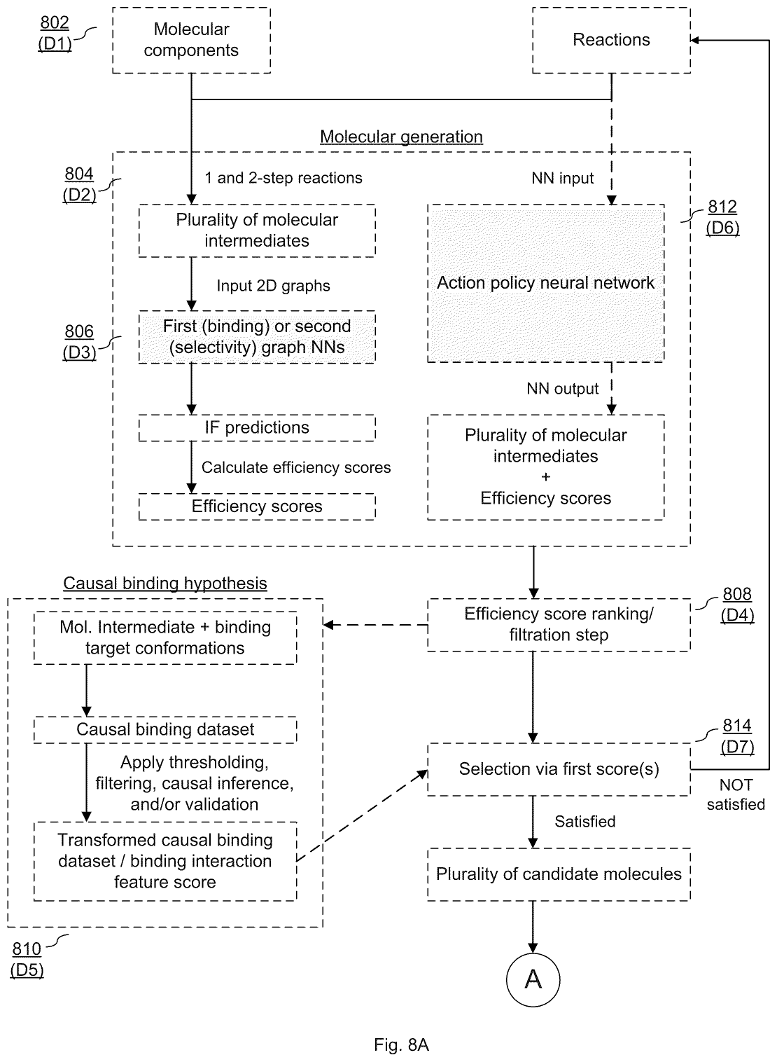

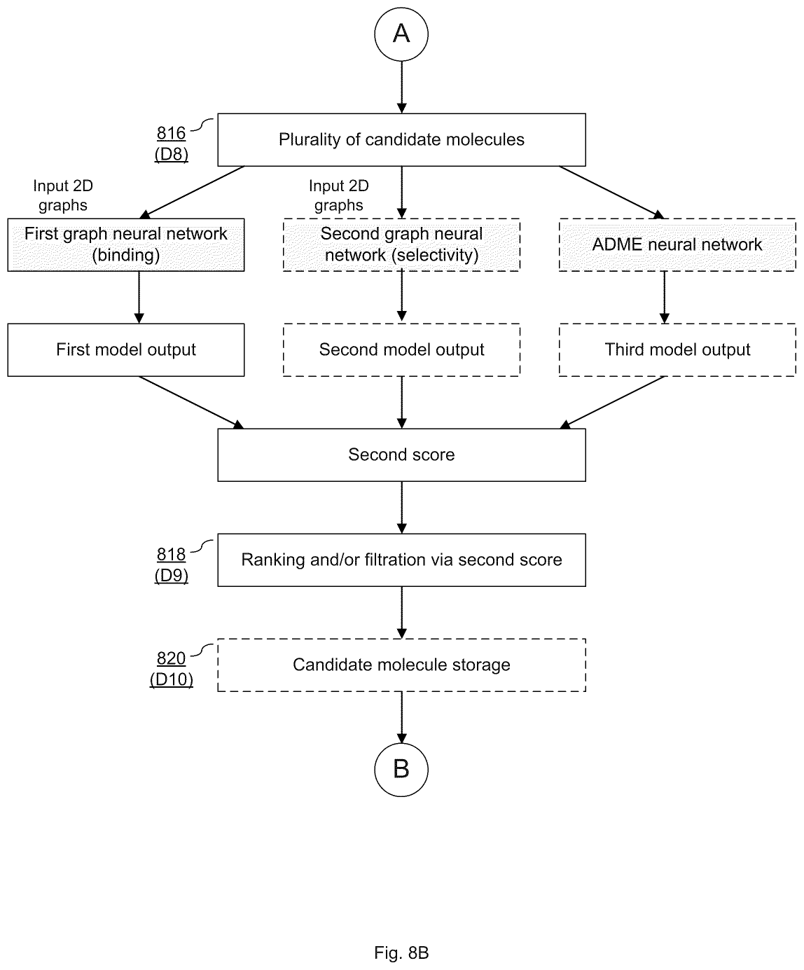

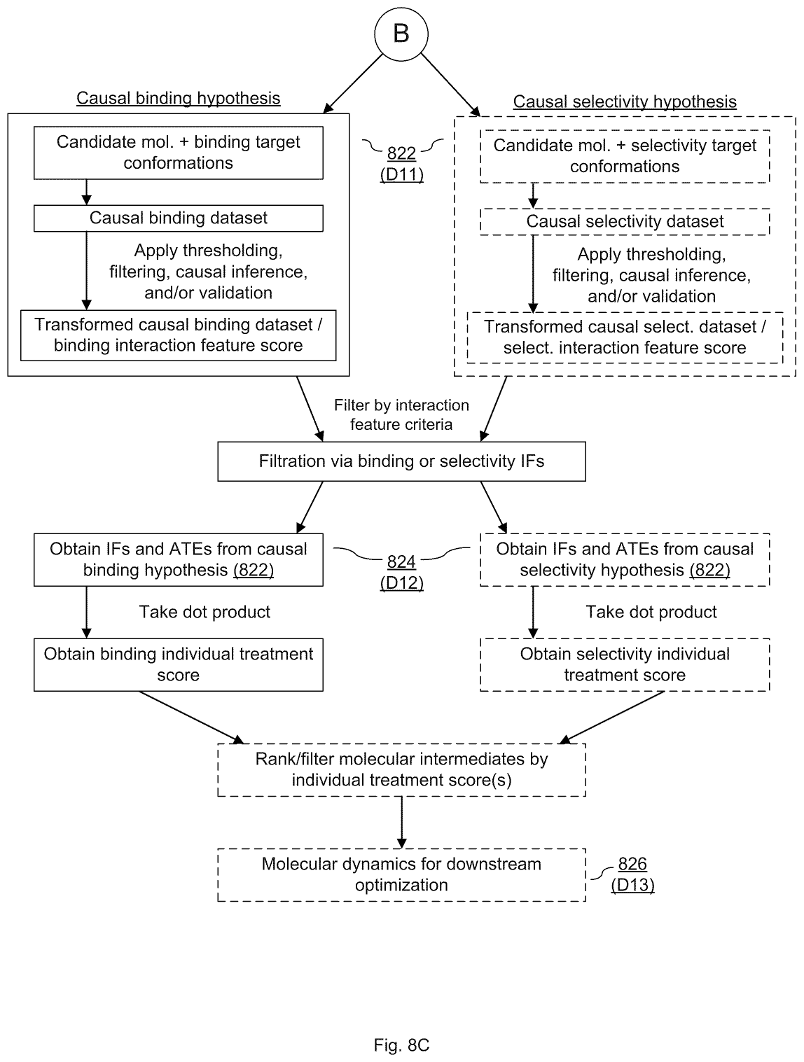

An example molecular generation process is shown as block 308 within a pipeline 300 for characterizing interactions between candidate molecules and target macromolecules, depicted in FIG. 3. Molecular generation process 308 is further detailed as a workflow in FIGS. 4D and 8A-C.

Referring to block 204, in some embodiments, the method further includes, prior to the selecting A), obtaining a plurality of molecular reactions and a plurality of molecular components (e.g., step D1 and block 802 in FIGS. 4D and 8A). For each respective molecular component in the plurality of molecular components, the respective molecular component is transformed using a corresponding one or more molecular reactions in the plurality of molecular reactions, thereby generating a plurality of molecular intermediates (e.g., step D2 and block 804). For each respective molecular intermediate in the plurality of molecular intermediates, a respective score is determined for the interaction between the respective molecular intermediate and the target macromolecule or the target macromolecule complex (e.g., step D3 and block 806). In some embodiments, one or more molecular intermediates are removed from the plurality of molecular intermediates, based on the respective score for the interaction between each respective molecular intermediate and the target macromolecule or the target macromolecule complex (e.g., step D4 and block 808). In some embodiments, the one or more molecular intermediates are not removed from the plurality of molecular intermediates.

In some embodiments, the plurality of molecular components comprises at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹ molecular components. In some embodiments, the plurality of molecular components comprises no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000 molecular components. In some embodiments, the plurality of molecular components consists of from 1000 to 100,000, from 10,000 to 1×10⁷, from 1×10⁶ to 1×10⁸, from 1×10⁸ to 1×10¹¹, or from 1×10⁹ to 1×10¹² molecular components. In some embodiments, the plurality of molecular components falls within another range starting no lower than 1000 molecular components and ending no higher than 1×10¹² molecular components.

In some embodiments, the plurality of molecular intermediates comprises at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹ molecular intermediates. In some embodiments, the plurality of molecular intermediates comprises no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000 molecular intermediates. In some embodiments, the plurality of molecular intermediates consists of from 1000 to 100,000, from 10,000 to 1×10⁷, from 1×10⁶ to 1×10⁸, from 1×10⁸ to 1×10¹¹, or from 1×10⁹ to 1×10¹² molecular intermediates. In some embodiments, the plurality of molecular intermediates falls within another range starting no lower than 1000 molecular intermediates and ending no higher than 1×10¹² molecular intermediates.

In some embodiments, the plurality of molecular reactions comprises at least 10, at least 50, at least 100, at least 500, or at least 1000 molecular reactions. In some embodiments, the plurality of molecular reactions comprises no more than 5000, no more than 1000, no more than 100, no more than 50, or no more than 20 molecular reactions. In some embodiments, the plurality of molecular reactions consists of from 10 to 100, from 50 to 200, from 100 to 500, or from 500 to 5000 molecular reactions. In some embodiments, the plurality of molecular reactions falls within another range starting no lower than 10 molecular reactions and ending no higher than 5000 molecular reactions.

In some embodiments, a respective molecular component in the plurality of molecular components consists of a single molecule. In some embodiments, a respective molecular component in the plurality of molecular components comprises a plurality of molecules. In some embodiments, the one or more molecular reactions applied during the transformation of each respective molecular component comprises at least 1, at least 2, at least 3, at least 4, at least 5, or at least 10 molecular reactions. In some embodiments, the one or more molecular reactions applied during the transformation of each respective molecular component comprises no more than 20, no more than 10, no more than 5, or no more than 2 molecular reactions. In some embodiments, the one or more molecular reactions applied during the transformation of each respective molecular component consists of from 1 to 5, from 2 to 10, or from 5 to 20 molecular reactions. In some embodiments, the one or more molecular reactions applied during the transformation of each respective molecular component falls within another range starting no lower than 1 molecular reaction and ending no higher than 20 molecular reactions.

In some embodiments, the plurality of molecular reactions comprises one or more reaction SMILES (Simplified Molecular Input Line Entry Specification). SMILES representations comprise at least two fundamental types of symbols for atoms and bonds, respectively. These symbols are used to specify a molecular graph for a respective molecule (e.g., using “nodes” and “edges”) and assign labels to the components of the graph that indicate, for example, the type of atom each node represents and/or the type of bond each edge represents.

In some embodiments, the plurality of molecular reactions comprises one or more reaction SMARTS (SMILES arbitrary target specification). SMARTS refers to a language that allows for the specification of molecular substructures using an extended set of rules. In particular, SMARTS uses atomic and bond symbols to specify a molecular graph, where the labels for the graph's nodes and edges (e.g., “atoms” and “bonds”) are extended to include “logical operators” and special atomic and bond symbols, thus allowing SMARTS atoms and bonds to be more general. Moreover, the SMARTS language can be used for the expression of molecular reactions (e.g., “reaction queries”). In some implementations, reaction queries are composed of optional reactant, agent, and product parts, which are separated by a “>” character. In such cases, the components of a reaction query match the corresponding roles within the reaction target. SMILES and SMARTS reactions are further disclosed, for example, in “SMARTS Theory Manual,” Daylight Chemical Information Systems, Santa Fe, New Mexico, available on the Internet at daylight.com/dayhtml/doc/theory/theory.smarts.html, which is hereby incorporated herein by reference in its entirety.

In some embodiments, the plurality of molecular reactions includes, but is not limited to, named reactions, organic synthesis reactions, protecting groups, total synthesis, Flow Chemistry, Green Chemistry, Microwave Synthesis, Multicomponent Reactions, Organocatalysis, and/or Sonochemistry. Alternatively or additionally, in some embodiments, the plurality of molecular reactions includes, but is not limited to, methyl esterification, hydrolysis of esters, amide synthesis, transamidation, oxidative amidation, Schmidt Reaction, Schotten-Baumann Reaction, Ugi Reaction, arylamine synthesis, Buchwald-Hartwig Reaction, Chan-Lam Coupling, Petasis Reaction, Ullmann Reaction, Hiyama Coupling, Kumada Coupling, Miyaura Borylation Reaction, Negishi Coupling, Stille Coupling, Suzuki Coupling, Sonogashira Coupling, Click Chemistry, Azide-Alkyne Cycloaddition, Copper-Catalyzed Azide-Alkyne Cycloaddition (CuAAC), Ruthenium-Catalyzed Azide-Alkyne Cycloaddition (RuAAC), Huisgen 1,3-Dipolar Cycloaddition, Synthesis of 1,2,3-Triazoles, epoxide synthesis, Jacobsen-Katsuki Epoxidation, Prilezhaev Reaction, Sharpless Epoxidation, Shi Epoxidation, and/or ring opening reactions of epoxides. Various molecular reactions are known in the art and are contemplated for use in the present disclosure. For instance, non-limiting examples of molecular reactions are further described in the Organic Chemistry Portal, available on the Internet at organic-chemistry.org.

In some embodiments, the transformation applies the one or more molecular reactions in a non-directed manner (e.g., via genetic algorithms) or in a directed manner (e.g., via reinforcement learning).

In some embodiments, the plurality of molecular intermediates serves as the collection of candidate molecules from which the plurality of candidate molecules is selected, and no further enumerations or molecular generation procedures are performed. Alternatively, in some embodiments, one or more additional molecular generation procedures are performed to refine existing molecular intermediates and/or to generate additional molecular intermediates to add to the collection of candidate molecules.

### Genetic Algorithm Exploration.

For example, referring to block 206, the method further includes, for each respective molecular intermediate in the plurality of molecular intermediates, transforming the respective molecular intermediate using a corresponding one or more molecular reactions in the plurality of molecular reactions, thereby generating the collection of candidate molecules. For each respective candidate molecule in the collection of candidate molecules, the respective first score for the interaction between the respective candidate molecule and the target macromolecule or the target macromolecule complex is determined. In some such embodiments, the selecting A) comprises removing one or more candidate molecules from the collection of candidate molecules based on the respective first score for the interaction between each respective candidate molecule and the target macromolecule or the target macromolecule complex. In some embodiments, the one or more candidate molecules are not removed from the collection of candidate molecules.

In some embodiments, only those molecular intermediates that satisfy each iteration of exploration (e.g., transforming, scoring, and removing) are retained and utilized for the subsequent iteration. Moreover, in some implementations, only those molecular intermediates that satisfy all iterations of exploration are selected as the plurality of candidate molecules.

Alternatively or additionally, in some implementations, molecular intermediates are added to the plurality of candidate molecules after one or more iterations of exploration. In some implementations, molecular intermediates are added to the plurality of candidate molecules after each iteration of exploration.

Reinforcement learning exploration.

Referring to block 208, in some embodiments, the method further includes, for each respective molecular intermediate in the plurality of molecular intermediates, responsive to inputting the respective molecular intermediate into a reinforcement learning model, retrieving, as output from the reinforcement learning model, a respective transformation of the respective molecular intermediate. In some embodiments, the respective transformation (i) represents a corresponding one or more molecular reactions in the plurality of molecular reactions, and (ii) is selected from a probability distribution of a plurality of transformations, for the respective molecular intermediate, associated with the corresponding one or more molecular reactions. Thus, the collection of candidate molecules is generated. In some embodiments, the method further includes, for each respective candidate molecule in the collection of candidate molecules, determining the respective first score for the interaction between the respective candidate molecule and the target macromolecule or the target macromolecule complex. In some embodiments, the selecting A) comprises removing one or more candidate molecules from the collection of candidate molecules based on the respective first score for the interaction between each respective candidate molecule and the target macromolecule or the target macromolecule complex. See, for example, step D6 and block 812 in FIGS. 4D and 8A. In some embodiments, the one or more candidate molecules are not removed from the collection of molecular intermediates.

In some embodiments, the one or more molecular reactions representing each respective transformation in the plurality of transformations comprises at least 1, at least 2, at least 3, at least 4, at least 5, or at least 10 molecular reactions. In some embodiments, one or more molecular reactions representing each respective transformation comprises no more than 20, no more than 10, no more than 5, or no more than 2 molecular reactions. In some embodiments, the one or more molecular reactions representing each respective transformation consists of from 1 to 5, from 2 to 10, or from 5 to 20 molecular reactions. In some embodiments, the one or more molecular reactions representing each respective transformation falls within another range starting no lower than 1 molecular reaction and ending no higher than 20 molecular reactions.

In some embodiments, for each respective molecular intermediate in the plurality of molecular intermediates, the corresponding probability distribution comprises at least 2, at least 3, at least 4, at least 5, at least 10, at least 20, at least 50, at least 100, at least 500, or at least 1000 transformations. In some embodiments, the plurality of transformations comprises no more than 10,000, no more than 1000, no more than 100, no more than 50, no more than 10, or no more than 5 transformations. In some embodiments, the plurality of transformations consists of from 2 to 10, from 5 to 50, from 30 to 500, or from 1000 to 10,000 transformations. In some embodiments, the plurality of transformations falls within another range starting no lower than 2 transformations and ending no higher than 10,000 transformations.

In some embodiments, prior to the inputting, the reinforcement learning model is trained.

Generally, a reinforcement learning system consists of four main elements—an agent, a policy, a reward signal, and a value function, where the behavior of the agent is defined in terms of the policy. Two forms of learning algorithms are possible. On-Policy learning algorithms evaluate and improve the same policy which is being used to select the agent's actions. Off-Policy learning algorithms evaluate and improve policies that are different from the policy being used for action selection. Reinforcement learning is further described, for example, in Sutton R S, Barto A G, “Reinforcement learning: an introduction,” IEEE Transactions on Neural Networks. 1998; 9(5):1054-1054, which is hereby incorporated herein by reference in its entirety.

In some embodiments, the reinforcement learning model is trained using molecular intermediates and/or candidate molecules obtained from one or more iterations of a molecular generation process, as described above. In some such embodiments, scores for each respective molecular intermediate or candidate molecule are used as training labels to assess the output from the reinforcement learning model. In some embodiments, the reinforcement learning model is trained using training molecules obtained using any suitable method, as will be apparent to one skilled in the art, including but not limited to the exploration procedures disclosed herein.

In some embodiments, the reinforcement learning model comprises a plurality (e.g., a third plurality) of at least 1000 parameters, where the training comprises, for each respective molecular component in the plurality of molecular components, performing a procedure comprising (i) obtaining a respective representation of a chemical structure of the respective molecular component and (ii) responsive to inputting the respective representation of the chemical structure of the respective molecular component into the reinforcement learning model, retrieving, as respective training output from the reinforcement learning model, a corresponding plurality of predicted transformations. In some such embodiments, each respective predicted transformation in the plurality of predicted transformations (a) represents a corresponding one or more molecular reactions in the plurality of molecular reactions, and (b) corresponds to a respective molecular intermediate in the plurality of molecular intermediates. The procedure further includes (iii) for each respective predicted transformation in the plurality of predicted transformations, using the respective first score for the interaction between the corresponding candidate molecule for the respective predicted transformation and the target macromolecule or the target macromolecule complex to adjust the third plurality of parameters. In some embodiments, the training includes repeating the (i) obtaining, (ii) retrieving, and (iii) adjusting for each iteration in a plurality of iterations, or until a performance of the reinforcement model satisfies a performance criterion.

In some embodiments, the plurality of parameters for the reinforcement learning model includes at least 10, at least 100, at least 1000, at least 10,000, at least 100,000, at least 1×10⁶, at least 1×10⁷, or more parameters. In some embodiments, the plurality of parameters for the reinforcement learning model includes no more than 1×10⁸, no more than 1×10⁷, no more than 1×10⁶, no more than 100,000, no more than 10,000, no more than 1000, or no more than 100 parameters. In some embodiments, the plurality of parameters for the reinforcement learning model consists of from 10 to 1000, from 100 to 100,000, from 10,000 to 1×10⁷, or from 1×10⁶ to 1×10⁸ parameters. In some embodiments, the plurality of parameters for the reinforcement learning model falls within another range starting no lower than 10 parameters and ending no higher than 1×10⁸ parameters.

In some embodiments, the exploration phase is performed for a number of iterations, for example, to refine existing molecular intermediates and/or to generate new molecular intermediates, for use as candidate molecules or as training molecules for the reinforcement learning model. Referring to block 210, in some embodiments, the transforming, determining, and selecting is repeated for each iteration in a plurality of iterations (e.g., step D7 and block 814 in FIGS. 4D and 8A).

In some embodiments, the plurality of iterations comprises at least 2, at least 3, at least 4, at least 5, at least 10, at least 20, at least 50, or at least 100 iterations. In some embodiments, the plurality of iterations comprises no more than 200, no more than 100, no more than 50, no more than 10, or no more than 5 iterations. In some embodiments, the plurality of iterations consists of from 2 to 10, from 5 to 50, from 30 to 100, or from 100 to 200 iterations. In some embodiments, the plurality of iterations falls within another range starting no lower than 2 iterations and ending no higher than 200 iterations.

### Causal Interaction Feature Scores.

In some embodiments, one or more molecules of the present disclosure (e.g., candidate molecules and/or molecular intermediates) are scored (e.g., for a first score 134). As described elsewhere herein, a respective score for a respective molecule can be used to determine whether the molecule is retained for further refinement, such as in the molecular generation procedure, or for further evaluation, such as in a selection or filtration step.

In some embodiments, a respective score for a respective molecule characterizes or otherwise indicates an interaction between the respective molecule and a target (or off-target) macromolecule or macromolecule complex. In some implementations, a respective score is a causal interaction feature score that is obtained using one or more interaction features associated with a conformation of the respective molecule when complexed to the target (or off-target) macromolecule or macromolecule complex. However, any suitable method for obtaining interaction scores is contemplated for use in the present disclosure, as will be apparent to one skilled in the art.

In some implementations, a respective score for a respective molecule is based at least on a count of interaction features for a conformation of the respective molecule when complexed to the target (or off-target) macromolecule or macromolecule complex. A count of interaction features can refer to a tally of the plurality of interaction features associated with the respective molecule, but can also refer to any weighted count or computation of causality over the plurality of interaction features.

Accordingly, in some implementations, a respective score is an absolute count, a weighted count, an individual treatment score (e.g., a dot product between an interaction feature vector and corresponding average treatment effects for each respective interaction feature in the interaction feature vector), a weighted individual treatment score, an efficiency score (e.g., a ratio of the number of interaction features for the respective molecule and the number of heavy atoms in the respective molecule), a weighted efficiency score, a diversity score (e.g., a measure of a diversity of interaction feature classes in a plurality of interaction features associated with the respective molecule when complexed to the macromolecule or macromolecule complex), and/or a weighted diversity score.

In some implementations, a weighted score gives greater import to one or more interaction features in a corresponding plurality of interaction features for a respective molecule, compared to other interaction features in the corresponding plurality of interaction features. In an example implementation, a weighted score gives greater weight to a first interaction feature that is selected as or known to be highly causal or associated with a particular property relevant to interaction (e.g., binding potency, selectivity, ADME properties, toxicity, etc.). In such an example implementations, the weighted score gives lesser weight to a second interaction feature that is selected as or known to be a covariate, confounder, or otherwise have lower causality for the particular property.

In some implementations, a weighted score is differentially weighted based on the presence or absence of one or more interaction features in a corresponding plurality of interaction features for a respective molecule. For instance, in some such implementations, a respective score for a respective molecule is predictive of binding when one or more interaction features, or classes thereof, in a first subset of interaction features is present in the corresponding plurality of interaction features for the respective molecule, and is not predictive of binding when none of the interaction features, or classes thereof, in the first subset of interaction features is present in the corresponding plurality of interaction features for the respective molecule. In other words, in some such implementations, a weighted score accounts for interaction features or feature classes that are selected as or known to be essential for a particular interaction property. Alternatively or additionally, in some implementations, a weighted score accounts for interaction features or feature classes that are selected as or known to be adverse or inhibitive to the particular interaction property. In some embodiments, a weighted score is determined by adjusting a corresponding attribute for each respective interaction feature by a weighting factor (e.g., 0.8, 0.2).

In some implementations, interaction feature classes include any of the feature classes disclosed elsewhere herein (see, e.g., the section entitled “Interaction features,” below), including but not limited to partial charge, H-bond acceptor, H-bond donor, aromatic ring, hydrophobic interaction, and/or other pharmacophores.

In some embodiments, a respective first score for the interaction between a respective molecule disclosed herein (e.g., a candidate molecule, a molecular component, a molecular intermediate, a training molecular component, and/or a training molecular intermediate) and a target macromolecule or the target macromolecule complex is obtained using a respective plurality of interaction features (e.g., a corresponding first, second, third, fourth, fifth, and/or any subsequent plurality of interaction features) obtained for a complex formed between the respective molecule and the target macromolecule or target macromolecule complex. In some embodiments, a respective first score is obtained for any suitable molecule complexed to the target macromolecule or target macromolecule complex, including but not limited to a candidate molecule, a molecular component, a molecular intermediate, a training molecular component, and/or a training molecular intermediate, as will be apparent to one skilled in the art.

One skilled in the art will appreciate that the interaction features (e.g., a first, second, third, fourth, fifth, or any subsequent plurality of interaction features) used for calculating a respective score (e.g., the first score) can be obtained using any suitable method, including but not limited to a causal binding hypothesis generation method, a causal selectivity hypothesis generation method, a graph neural network for binding, and/or a graph neural network for selectivity, as disclosed herein. See, for example, the sections entitled “Causal binding hypothesis generation,” “Causal selectivity hypothesis generation,” and Evaluating molecules via machine learning models,” below.

For example, as illustrated in FIGS. 4D and 8A, in some embodiments, the first score is an efficiency score. In some embodiments, the method further includes, prior to the selecting A), for each respective candidate molecule in the collection of candidate molecules, responsive to inputting a two-dimensional molecular graph of the respective candidate molecule into the first model, retrieving, as output from the first model, a corresponding first plurality of interaction features for a complex formed between the respective candidate molecule and the target macromolecule or the target macromolecule complex. The first plurality of interaction features for the respective candidate molecule is tallied, thereby obtaining a corresponding interaction feature count. A number of heavy atoms in the respective candidate molecule is tallied, thereby obtaining a corresponding heavy atom count. The respective first score for the interaction between the respective candidate molecule and the target macromolecule or the target macromolecule complex is calculated as a ratio between (i) the corresponding interaction feature count and (ii) the corresponding heavy atom count.

In some embodiments, the collection of candidate molecules is ranked based on the respective first score for the interaction between each respective candidate molecule in the collection of candidate molecules and the target macromolecule or the target macromolecule complex.

In some embodiments, the method includes determining multiple scores for a respective molecule (e.g., a molecular intermediate and/or a candidate molecule). In some embodiments, two or more types of scores are obtained for a respective molecule within a single iteration of molecular generation. Alternatively or additionally, in some embodiments, a respective molecule is iteratively scored after each iteration of molecular generation. In some embodiments, multiple iterations of scoring, evaluation, and/or removing are performed, in order to further refine the plurality of molecular intermediates and/or the plurality of candidate molecules.

An example implementation for using the causal binding hypothesis generation procedure for obtaining an additional score for selection of molecular intermediates during molecular generation is illustrated in step D5 and block 810 in FIGS. 4D and 8A.

In some embodiments, the method further includes, prior to the selecting A), for each respective candidate molecule in the collection of candidate molecules, determining a corresponding plurality (e.g., a fifth plurality) of interaction features, where each respective interaction feature in the fifth plurality of interaction features is associated with a binding affinity between the respective candidate molecule and the target macromolecule or the target macromolecule complex. One or more candidate molecules are removed, from the collection of candidate molecules, based at least on a count of interaction features in the fifth plurality of interaction features for each respective candidate molecule in the collection of candidate molecules. In some embodiments, the one or more candidate molecules are not removed from the collection of molecular intermediates.

In some embodiments, referring again to block 202, the selecting A) is performed when the first score fails to satisfy a criterion value. In some implementations, criterion values and their conditions for satisfaction are dependent on the type of score being obtained. For instance, when the score is an absolute count of interaction features causal for binding, the candidate molecule fails to satisfy the criterion when the absolute count is less than a threshold number of interaction features deemed to be sufficient for potent binding (e.g., less than 100, less than 50, less than 20, less than 10, etc.). Alternatively or additionally, when the score is an individual treatment score calculated as a dot product of an interaction feature vector and corresponding average treatment effects (ATEs) of the respective interaction features, the candidate molecule fails to satisfy the criterion when the individual treatment score is greater than a threshold value (e.g., greater than −1, greater than −0.5, greater than −0.1, greater than 0, etc.). In general, because the individual treatment score is calculated using the ATEs of individual interaction features, and because ATEs are representative of the Gibbs free energy of a particular conformation of the respective molecule complexed with the target or off-target macromolecule or macromolecule complex, higher individual treatment scores are predictive of poor overall binding affinity or specificity.

In some embodiments, a criterion value is determined based on a predetermined hypothesis or prior.

In some embodiments, a criterion value is determined based on one or more predetermined parameters known to be associated, highly causal, or necessary with a particular property relevant to interaction (e.g., binding potency, selectivity, ADME properties, toxicity, etc.). Predetermined parameters can be obtained from literature, published data, and/or experimental results. For instance, in some implementations, cutoff thresholds for ADME properties are determined based on outcomes of historical data on other molecules.

In some embodiments, a criterion value is determined based on one or more parameters for a control molecule known to exhibit target properties. For instance, in some implementations, a criterion value is determined by identifying one or more lead candidates or tool compounds that have been observed to exhibit target levels of binding, specificity, ADME properties, and/or drug-likeness. A lead candidate or tool compound is scored, using any one or more of the scoring methods disclosed above. The values obtained from the scoring methods are then used as a baseline threshold to establish the criterion value for further assessment of other candidate molecules. In some embodiments, a value obtained for a lead candidate or tool compound is used to establish the criterion value without alteration. Alternatively, in some embodiments, a value obtained for a lead candidate or tool compound is used to establish the criterion value is adjusted in order to establish the criterion value (e.g., to encourage identification of candidate molecules having improved performance over the control molecules).

In some embodiments, a criterion value is determined by an optimization process. In some implementations, the optimization process includes, for each respective candidate molecule in the plurality of candidate molecules, classifying the respective candidate molecule as an active or inactive molecule, thus obtaining a first subset of active molecules and a second subset of inactive molecules. Determination of active and inactive molecules is described in further detail elsewhere herein (see, e.g., the section entitled “Causal binding hypothesis generation,” below). The respective criterion value is then initialized randomly or using knowledge-based or historical data. Each candidate molecule in the plurality of candidate molecules is scored, using the criterion value as a cutoff threshold, and the plurality of candidate molecules is assessed to determine whether the criterion value achieved accurate separate of active molecules from inactive molecules. The criterion value is adjusted and the scoring, assessing, and adjusting is repeated until a maximal separation of active molecules and inactive molecules is achieved.

In some embodiments, the selecting A) includes ranking and selecting the top ranked N candidate molecules in the collection of candidate molecules. In some embodiments, N is at least at least 5, at least 10, at least 20, at least 50, at least 100, at least 500, or at least 1000. In some embodiments, N is no more than 5000, no more than 1000, no more than 100, no more than 50, or no more than 10. In some embodiments, N is from 5 to 50, from 30 to 500, or from 1000 to 5000. In some embodiments, N falls within another range starting no lower than 2 and ending no higher than 5000.

In some embodiments, the selecting A) includes ranking and selecting the top ranked M percent of candidate molecules in the collection of candidate molecules. In some embodiments, M is at least 5, at least 10, at least 20, or at least 50. In some embodiments, M is no more than 80, no more than 50, no more than 20, or no more than 10. In some embodiments, M is from 2 to 10, from 8 to 40, or from 40 to 80. In some embodiments, M falls within another range starting no lower than 2 and ending no higher than 80.

### Evaluating Molecules Via Machine Learning Models.

Returning again to FIGS. 2A-J, referring to block 211, the method 200 further includes B) performing a first filtering step for the plurality of candidate molecules 132. The first filter step comprises, for each respective candidate molecule 132 in the plurality of candidate molecules, responsive to inputting a two-dimensional molecular graph 142 of the respective candidate molecule into a first model 152-1, retrieving, as output from the first model, a corresponding first plurality of interaction features 154-1 for a complex formed between the respective candidate molecule 132 and the target macromolecule or the target macromolecule complex 122. Alternatively or additionally, responsive to inputting the two-dimensional molecular graph 142 of the respective candidate molecule into a second model 152-2, there is retrieved, as output from the second model, a corresponding second plurality of interaction features 154-2 for a complex formed between the respective candidate molecule 132 and an off-target macromolecule or off-target macromolecule complex 124, other than the target macromolecule or target macromolecule complex 122.

At least the first plurality of interaction features 154-1 or the second plurality of interaction features 154-2 is used to obtain a corresponding second score 136 for the interaction between the respective candidate molecule 132 and the target macromolecule or the target macromolecule complex 122. One or more candidate molecules 132 are removed from the plurality of candidate molecules based on an evaluation of the corresponding second score 136 for each respective candidate molecule in the plurality of candidate molecules. In some embodiments, the first model 152-1 comprises a first plurality of at least 1000 parameters 156 and the second model 152-2 comprises a second plurality of at least 1000 parameters 156. In some embodiments, the one or more candidate molecules are not removed from the plurality of molecular intermediates.

Optionally, in some embodiments, one or more molecular candidates are stored in a molecular database for further evaluation. See, for example, steps D8-D10 in FIG. 4D and blocks 816-820 in FIG. 8B.

### First Model.

Referring to block 212, in some embodiments, the first model is a first graph neural network. In some embodiments, input to the model includes, for each respective candidate molecule in the plurality of candidate molecules, a two-dimensional molecular graph of the respective candidate molecule.

Graph Neural Networks (GNNs) are an effective framework for representation learning of graphs. GNNs follow a neighborhood aggregation scheme, where the representation vector of a node is computed by recursively aggregating and transforming representation vectors of its neighboring nodes. After k iterations of aggregation, a node is represented by its transformed feature vector, which captures the structural information within the node's k-hop neighborhood. The representation of an entire graph can then be obtained through pooling, for example, by summing the representation vectors of all nodes in the graph. Input to a GNN includes molecular graphs, labeled graphs where the vertices and edges represent the atoms and bonds of the molecule, respectively. Graph neural networks and molecular graphs are further described, for example, in Xu et al., “How powerful are graph neural networks?” ICLR 2019, arXiv:1810.00826v3, which is hereby incorporated herein by reference in its entirety.

GNN variants for both node and graph classification tasks are known in the art. For example, in some embodiments, the first model is a graph convolutional neural network. Nonlimiting examples of graph convolutional neural networks are disclosed in Behler Parrinello, 2007, “Generalized Neural-Network Representation of High Dimensional Potential-Energy Surfaces,” Physical Review Letters 98, 146401; Chmiela et al., 2017, “Machine learning of accurate energy-conserving molecular force fields,” Science Advances 3(5):e1603015; Schütt et al., 2017, “SchNet: A continuous-filter convolutional neural network for modeling quantum interactions,” Advances in Neural Information Processing Systems 30, pp. 992-1002; Feinberg et al., 2018, “PotentialNet for Molecular Property Prediction,” ACS Cent. Sci. 4, 11, 1520-1530; and Stafford et al., “AtomNet PoseRanker: Enriching Ligand Pose Quality for Dynamic Proteins in Virtual High Throughput Screens,” chemrxiv.org/engage/chemrxiv/article-details/614b905e39ef6a1c36268003, each of which is hereby incorporated by reference.

In some embodiments, the first model is a neural network (e.g., a multi-layer perceptron, a fully connected neural network, a partially connected neural network, etc.), a support vector machine, a Naive Bayes algorithm, a nearest neighbor algorithm, a boosted trees algorithm (e.g., XGBoost, LightGBM), a random forest algorithm, a decision tree algorithm, a logistic regression algorithm, a linear model, a linear regression algorithm, and/or any combination thereof. Various other model architectures are possible for use in obtaining, for a respective molecule or a representation thereof, a corresponding plurality of interaction features for a complex formed between the respective molecule and a macromolecule or macromolecule complex, as will be apparent to one skilled in the art.

In some implementations, the model is any of the model architectures disclosed herein, where the input to the model varies depending on the architecture selected for use, as will be apparent to one skilled in the art. For example, in some embodiments, input to the model includes, for each respective candidate molecule in the plurality of candidate molecules, a respective molecular fingerprint of a chemical structure of the respective candidate molecule.

Referring to block 214, in some embodiments, each respective interaction feature in the first plurality of interaction features is predicted by the first model to be causal for a binding affinity between the respective candidate molecule and the target macromolecule or the target macromolecule complex.

Referring to block 216, the method further includes, prior to the performing B), training the first model.

As described above, any number of architectures or systems are contemplated for use for characterizing an interaction between a candidate molecule and a target macromolecule or the target macromolecule complex. Before each such architecture or system can be used to characterize an interaction between a candidate molecule and the target macromolecule or macromolecule complex, it is trained against training molecules. A significant difference between a candidate molecule and training molecules is that the training molecules are labeled (e.g., with interaction feature vectors against the target macromolecule or macromolecule complex obtained from a candidate binding hypothesis and/or a candidate selectivity hypothesis, etc.) and such labeling is used to train the first model, second model, third model, and/or any subsequent models of the present disclosure, or any component or ensemble models thereof, whereas each candidate molecule is either not labeled or the labels are not used and the first model, second model, third model, and/or any subsequent models of the present disclosure, or any component or ensemble models thereof, are used to characterize an interaction between each candidate molecule and the target macromolecule or macromolecule complex. In other words, the training molecules are already characterized by labels (characterization of the interaction between the training molecules and the target macromolecule or macromolecule complex), and such characterization is used to train the models of the present disclosure to characterize an interaction between the candidate molecules and the target macromolecule or macromolecule complex. The interaction between the candidate molecules and the target macromolecule or macromolecule complex are typically not characterized prior to application of the first model, second model, third model, and/or any subsequent models of the present disclosure, or any component or ensemble models thereof. In typical embodiments, the characterizations of the interactions between the training molecules and the target macromolecule or macromolecule complex that are available includes interaction feature vectors against the target macromolecule or macromolecule complex obtained from a candidate binding hypothesis and/or a candidate selectivity hypothesis. In some embodiments, each respective interaction feature vector for a respective training molecule includes, for each respective interaction feature in the interaction feature vector, a corresponding geometric representation, attribute value, and/or representation thereof that indicates whether, or to what degree, the respective interaction feature is causal for a binding affinity and/or selectivity between the respective training molecule and the target macromolecule or the target macromolecule complex.

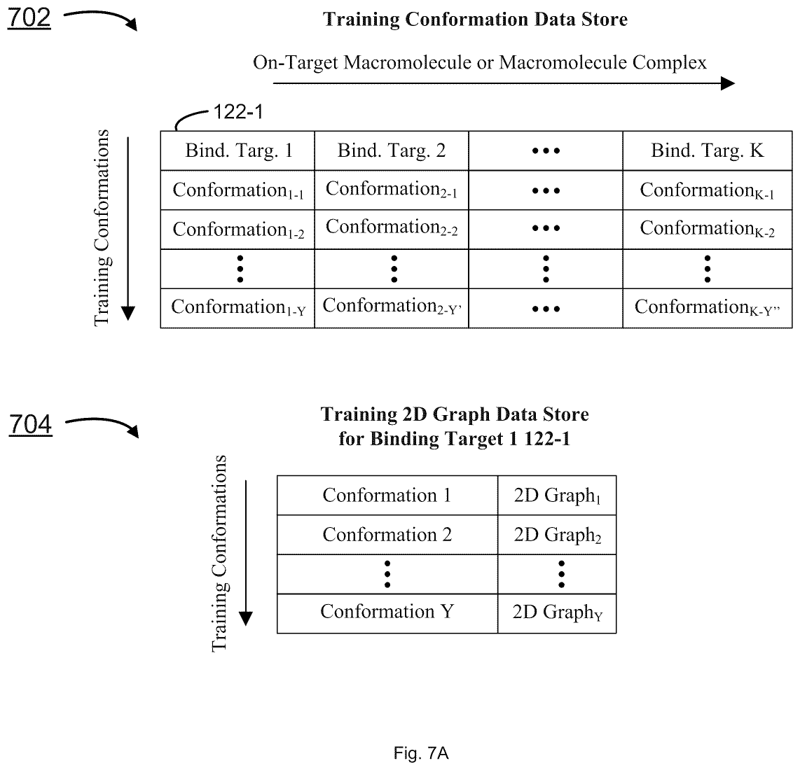

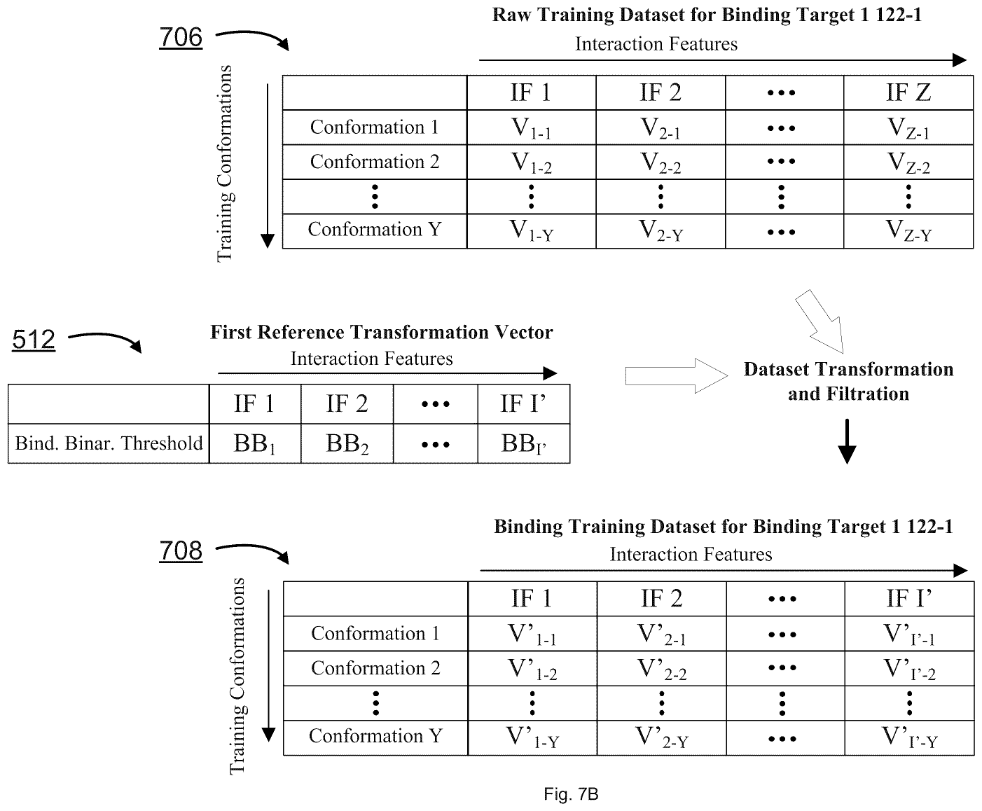

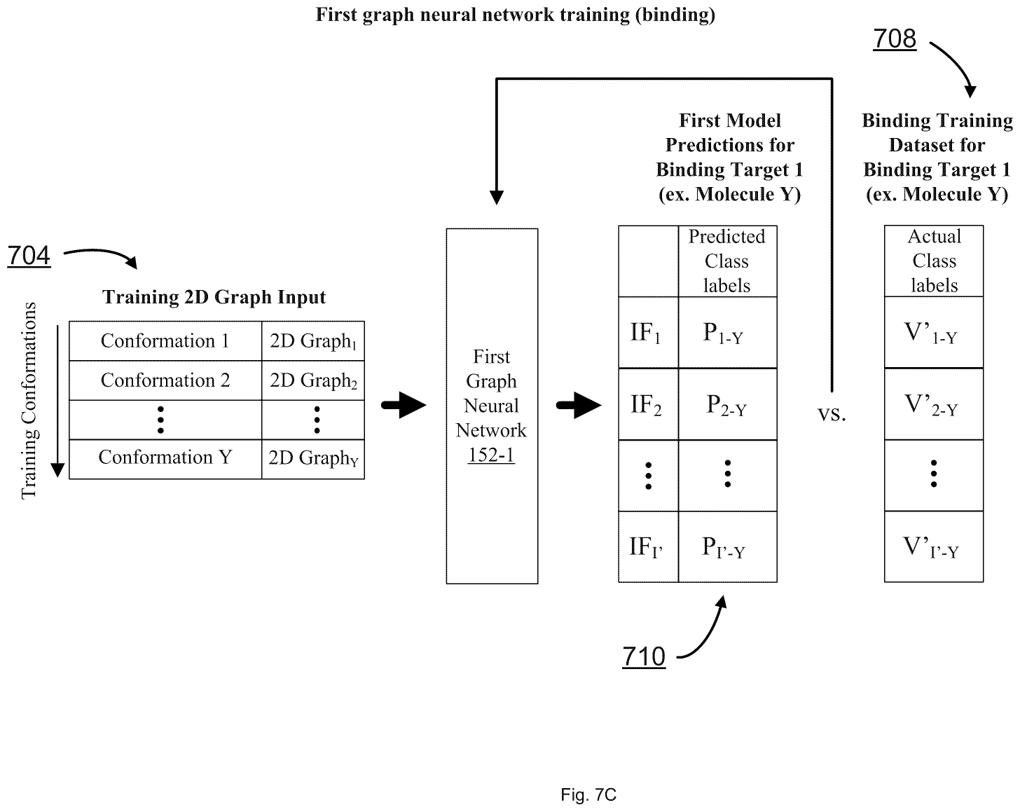

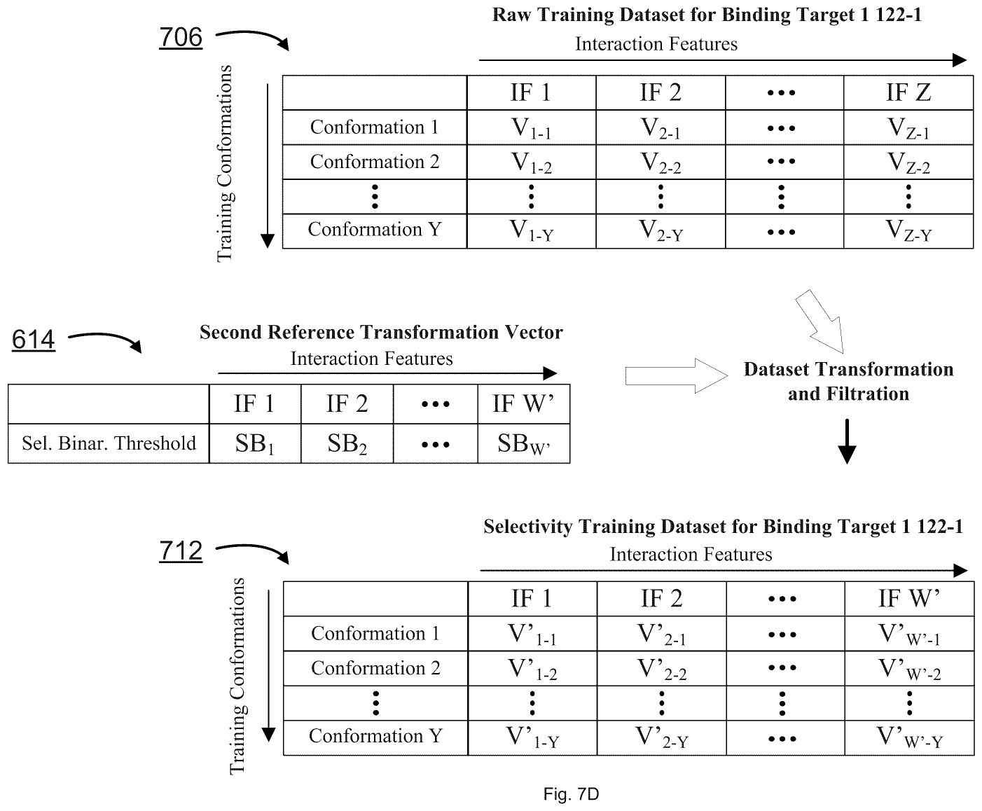

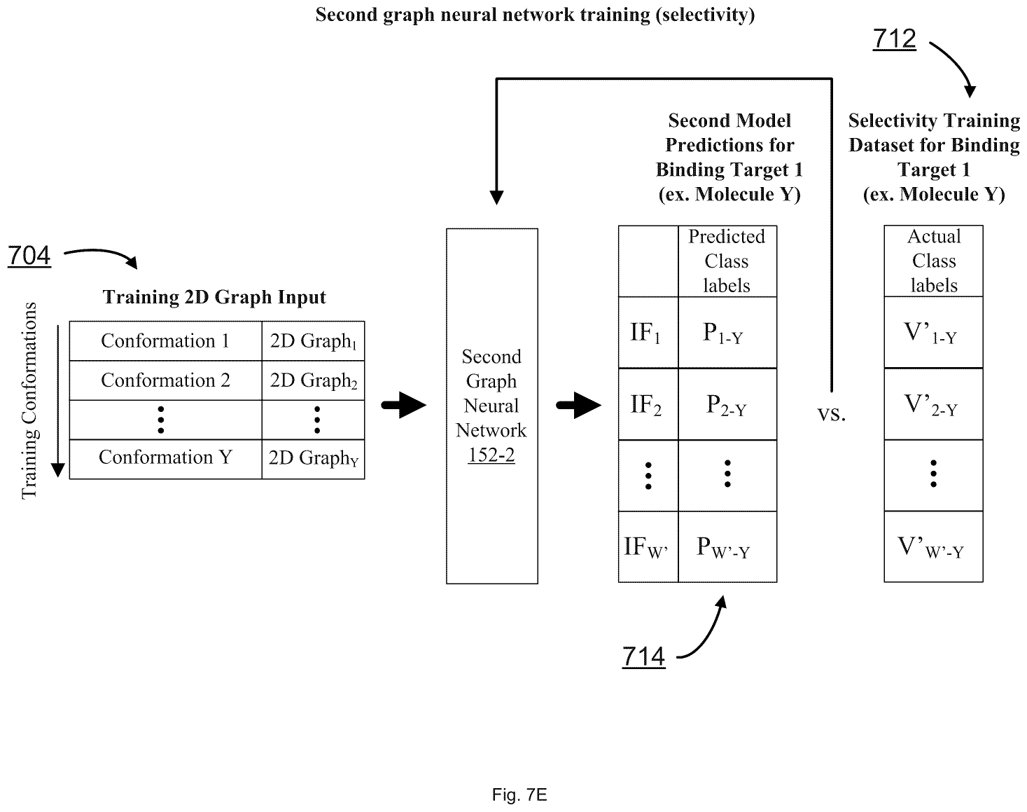

In some embodiments, a model in accordance with the present disclosure, such as the first and/or second model collectively depicted in FIGS. 7A-E, is trained to output, responsive to receiving as input a two-dimensional molecule graph of a respective molecule, a corresponding plurality of interaction features for a complex formed between the respective molecule and a macromolecule or the macromolecule complex. For instance, in some embodiments, the first model outputs, responsive to receiving as input a two-dimensional molecule graph of a respective training molecule, a corresponding first plurality of interaction features for a complex formed between the respective training molecule and the target macromolecule or the target macromolecule complex. Alternatively or additionally, in some embodiments, the second model outputs, responsive to receiving as input a two-dimensional molecule graph of the respective training molecule, a corresponding second plurality of interaction features for a complex formed between the respective training molecule and an off-target macromolecule or off-target macromolecule complex, other than the target macromolecule or target macromolecule complex. In some embodiments, a respective model outputs, for each respective interaction feature in the corresponding plurality of interaction features, a corresponding geometric representation, attribute value, and/or representation thereof that indicates whether, or to what degree, the respective interaction feature is causal for a binding affinity and/or selectivity between the respective training molecule and the target macromolecule or the target macromolecule complex. As illustrated in FIGS. 7A-E, for each respective training molecule in a plurality of training molecules, the output from the model (e.g., interaction features, geometric representations, attribute values, and/or representations thereof) serves as a predicted class label, while the training molecule labels (e.g., interaction feature vectors against the target macromolecule or macromolecule complex obtained from a candidate binding hypothesis and/or a candidate selectivity hypothesis) serves as an actual class label.

Errors in the predicted class labels (e.g., the plurality of interaction features outputted by the systems of the present disclosure), as verified against the actual class labels, are then back-propagated through the parameters of the each of the models of the systems of the present disclosure (e.g., first model, second model, third model, and/or any subsequent models, or any component or ensemble models thereof) in order to train the system. In an example embodiment, a model of the present disclosure is trained against the errors in the predicted class labels made by the model, in view of the actual class labels, by stochastic gradient descent with the AdaDelta adaptive learning method (Zeiler, 2012 “ADADELTA: an adaptive learning rate method,” CoRR, vol. abs/1212.5701, which is hereby incorporated by reference), and the back propagation algorithm provided in Rumelhart et al., 1988, “Neurocomputing: Foundations of research,” ch. Learning Representations by Back-propagating Errors, pp. 696-699, Cambridge, MA, USA: MIT Press, which is hereby incorporated by reference.

In some embodiments, model training involves modifying the parameters of one or more models, or any components or ensembles thereof. In some embodiments, the parameters are further constrained with various forms of regularization such as L1, L2, weight decay, and dropout.

In an embodiment, any one or more of the models disclosed herein, or any components or ensembles thereof, optionally, where training data is labeled (e.g., with interaction features, geometric representations, attribute values, and/or representation thereof), have their parameters (e.g., weights) tuned (adjusted to potentially minimize the error between the system's predicted class labels and the training data's actual class labels). Various methods are contemplated for minimizing the error function, such as gradient descent methods, including but not limited to log-loss, sum of squares error, hinge-loss methods. In some implementations, these methods include second-order methods or approximations such as momentum, Hessian-free estimation, Nesterov's accelerated gradient, adagrad, etc. In some implementations, model training further includes unlabeled generative pretraining and/or labeled discriminative training.

An example model training process is shown as block 306 within pipeline 300 for characterizing interactions between candidate molecules and target macromolecules, depicted in FIG. 3. Model training process 306 is further depicted as a workflow in FIG. 4C, with reference to data structures 702, 704, 706, 708, 710, 712, and 714 in FIGS. 7A-E.

Referring to block 218, in some embodiments, the training comprises, for each respective training molecule in a first plurality of at least 100,000 training molecules, performing a procedure comprising (i) obtaining a respective training two-dimensional molecular graph of a chemical structure of the respective training molecule. The procedure further includes (ii) responsive to inputting the respective training two-dimensional molecular graph of the chemical structure of the respective training molecule into the first model, retrieving, as respective training output from the first model, for each respective interaction feature in a first collection of interaction features, a corresponding training predicted label that indicates whether, or to what degree, the respective interaction feature is causal for a binding affinity between the respective training molecule and the target macromolecule or the target macromolecule complex. The procedure further includes (iii) applying a respective difference to a loss function to obtain a respective output of the loss function, wherein the respective difference is between, for each respective interaction feature in the first collection of interaction features, (a) the corresponding training predicted label from the first model and (b) a corresponding reference label that indicates whether, or to what degree, the respective interaction feature is causal for a binding affinity between the respective training molecule and the target macromolecule or the target macromolecule complex. The procedure further includes (iv) using the respective output of the loss function to adjust the first plurality of parameters.

In some embodiments, the first plurality of training molecules includes at least 100, at least 500, at least 1000, at least 5000, at least 10,000, at least 100,000, at least 1×10⁶, at least 5×10⁶, at least 1×10⁷, at least 1×10⁸, or at least 5×10⁸ training molecules. In some embodiments, the first plurality of training molecules includes no more than 1×10¹, no more than 1×10⁸, no more than 1×10⁷, no more than 1×10⁶, no more than 500,000, no more than 100,000, no more than 10,000, or no more than 1000 training molecules. In some embodiments, the first plurality of training molecules consists of from 100 to 1000, from 1000 to 100,000, from 10,000 to 5×10⁶, from 1×10⁶ to 1×10⁷, from 1×10⁷ to 1×10⁸, or from 1×10⁸ to 1×10⁸ training molecules. In some embodiments, the first plurality of training molecules falls within another range starting no lower than 100 training molecules and ending no higher than 1×10¹ training molecules.

Referring to block 220, in some embodiments, the method further includes, prior to the training, for each respective training molecule in the first plurality of training molecules, obtaining a corresponding three-dimensional pose of the respective training molecule complexed to the target macromolecule or the target macromolecule complex. A corresponding interaction feature vector is determined for the respective training molecule comprising, for each respective interaction feature in the first collection of interaction features, a respective geometric representation of the respective interaction feature in the corresponding three-dimensional pose of the respective training molecule complexed to the target macromolecule or the target macromolecule complex. In some embodiments, the corresponding interaction feature vector for the respective training molecule further comprises, for each respective interaction feature in the first collection of interaction features, a corresponding attribute value of the respective interaction feature. In some embodiments, the corresponding value is a scalar value. The corresponding interaction feature vector is transformed using a first reference transformation vector, thereby obtaining, for each respective interaction feature in the first collection of interaction features, the corresponding reference label that indicates whether, or to what degree, the respective interaction feature is causal for a binding affinity between the respective training molecule and the target macromolecule or the target macromolecule complex.

In some embodiments, for each respective training molecule in the first plurality of training molecules, the corresponding three-dimensional pose of the respective training molecule complexed to the target macromolecule or the target macromolecule complex is selected from a set of corresponding three-dimensional poses. In some embodiments, the corresponding three-dimensional pose is selected based on a docking program, as described elsewhere herein (see, e.g., the section entitled “Definitions: Pose,” above). In some implementations, the corresponding three-dimensional pose is selected as the best pose from a set of possible three-dimensional poses for the respective training molecule (e.g., based on a measure of binding affinity and/or specificity associated with the respective pose).

In some embodiments, a respective geometric representation and/or attribute value is transformed using a segmentation algorithm (e.g., a thresholding and/or binarization algorithm). In some embodiments, the transformation further includes filtering (e.g., removing), from the first collection of interaction features, each respective interaction feature that is not also included in the first reference transformation vector. In some embodiments, one or more interaction features are not removed from the first collection of interaction features.

For instance, referring to block 222, in some embodiments, the transforming applies a dimension reduction to the geometric representation of each respective interaction feature in the corresponding interaction feature vector for the respective training molecule. In some embodiments, the transforming applies a dimension reduction to the corresponding attribute value of each respective interaction feature in the corresponding interaction feature vector for the respective training molecule. In some implementations, for each respective interaction feature in the corresponding interaction feature vector, the transforming reduces a number of dimensions for the geometric representation from 3 to 1.

Referring to block 224, in some embodiments, the first reference transformation vector comprises, for each respective interaction feature in the first collection of interaction features, a corresponding binarization threshold for the respective interaction feature. In some embodiments, the transforming generates, for each respective interaction feature in the corresponding interaction feature vector for the respective training molecule, a corresponding binary value for the respective interaction feature. For instance, in some implementations, the transforming transforms, for each respective interaction feature in the corresponding interaction feature vector, a scalar value corresponding to the geometric representation and/or the attribute value of the respective interaction feature from a scalar value to a binary value.

Methods of obtaining reference transformation vectors (e.g., the first and/or the second reference transformation vector) are further disclosed elsewhere herein (see, e.g., the sections entitled “Causal binding hypothesis generation” and “Causal selectivity hypothesis generation,” below).

### Second Model

Referring to block 226, in some embodiments, the second model is a second graph neural network.

Referring to block 228, in some embodiments, each respective interaction feature in the second plurality of interaction features is predicted by the second model to be causal for a binding selectivity between the respective candidate molecule and the target macromolecule or the target macromolecule complex.

As described elsewhere herein, in some embodiments, a target macromolecule or macromolecule complex is a macromolecule or complex of interest as a primary binding target for a respective molecule, whereas an off-target macromolecule or macromolecule complex is a macromolecule or complex that is not the primary binding target but is associated with the target macromolecule or macromolecule complex as a candidate for off-target interactions. See, for example, the section entitled “Definitions: Target,” above.

In some embodiments, the target macromolecule or target macromolecule complex is associated with a set of off-target macromolecules or macromolecule complexes. In some embodiments, the set of off-target macromolecules or macromolecule complexes comprises at least 1, at least 2, at least 3, at least 4, at least 5, at least 10, at least 15, or at least 20 off-target macromolecules or macromolecule complexes. In some embodiments, the set of off-target macromolecules or macromolecule complexes comprises no more than 50, no more than 20, no more than 10, or no more than 5 off-target macromolecules or macromolecule complexes. In some embodiments, the set of off-target macromolecules or macromolecule complexes consists of from 1 to 5, from 2 to 10, from 5 to 20, or from 20 to 50 off-target macromolecules or macromolecule complexes. In some embodiments, the set of off-target macromolecules or macromolecule complexes falls within another range starting no lower than 1 and ending no higher than 50 off-target macromolecules or macromolecule complexes.

In some embodiments, the target macromolecule or target macromolecule complex is not associated with an off-target macromolecule or macromolecule complex.

In some embodiments, each respective target macromolecule or target macromolecule complex in a plurality of target macromolecules or macromolecule complexes has a same or different number of associated off-target macromolecules or macromolecule complexes.

In some embodiments, the second model generates, as output, the corresponding second plurality of interaction features for a complex formed between the respective candidate molecule and an off-target macromolecule or off-target macromolecule complex (e.g., where the second model is trained to generate outputs for a single off-target macromolecule or macromolecule complex). In some embodiments, the second model generates, as output, for each respective an off-target macromolecule or macromolecule complex in a plurality of off-target macromolecules or macromolecule complexes, a corresponding second plurality of interaction features for a respective complex formed between the candidate molecule and the respective off-target macromolecule or off-target macromolecule complex (e.g., where the second model is trained to generate outputs associated with a plurality of off-target macromolecules or macromolecule complexes). In some implementations, the method further includes obtaining a plurality of selectivity models, each respective model in the plurality of selectivity models associated with a different respective off-target macromolecule or macromolecule complex, and using each respective selectivity model in the plurality of selectivity models to generate a corresponding plurality of interaction features that is predicted by the respective selectivity model to be causal for a binding selectivity between the respective candidate molecule and the target macromolecule or the target macromolecule complex, relative to the respective off-target macromolecule or macromolecule complex. In some embodiments, the second model is an ensemble model comprising a plurality of component models. In some embodiments, the second model aggregates outputs (e.g., each respective plurality of interaction features in a set of pluralities of interaction features) obtained for each different respective off-target macromolecule or macromolecule complex in the set of off-target macromolecules or macromolecule complexes.

In some embodiments, the method further includes, prior to the performing B), training the second model.

In some implementations, any of the model architectures and/or model training embodiments disclosed herein for a first respective model are contemplated for use for a second, third, and/or any subsequent model, or any components or ensembles thereof, as will be apparent to one skilled in the art.

For example, in some implementations, the training comprises, for each respective training molecule in a second plurality of at least 100,000 training molecules, performing a procedure comprising (i) obtaining a respective training two-dimensional molecular graph of a chemical structure of the respective training molecule. The procedure further includes (ii) responsive to inputting the respective training two-dimensional molecular graph of the chemical structure of the respective training molecule into the second model, retrieving, as respective training output from the second model, for each respective interaction feature in a second collection of interaction features, a corresponding training predicted label that indicates whether, or to what degree, the respective interaction feature is causal for a binding selectivity between the respective training molecule and the target macromolecule or the target macromolecule complex. The procedure further includes (iii) applying a respective difference to a loss function to obtain a respective output of the loss function, where the respective difference is between, for each respective interaction feature in the second collection of interaction features, (a) the corresponding training predicted label from the second model and (b) a corresponding reference label that indicates whether, or to what degree, the respective interaction feature is causal for a binding selectivity between the respective training molecule and the target macromolecule or the target macromolecule complex. The procedure further includes (iv) using the respective output of the loss function to adjust the second plurality of parameters.

In some embodiments, the second plurality of training molecules includes all or a portion of the first plurality of training molecules. In some embodiments, the second plurality of training molecules does not share any training molecules in common with the first plurality of training molecules. In some implementations, any of the embodiments disclosed herein for a first plurality of training molecules are contemplated for use for a second, third, and/or any subsequent plurality of training molecules, as will be apparent to one skilled in the art.

For instance, in some embodiments, the second plurality of training molecules includes at least 100, at least 500, at least 1000, at least 5000, at least 10,000, at least 100,000, at least 1×10⁶, at least 5×10⁶, at least 1×10⁷, at least 1×10⁸, or at least 5×10⁸ training molecules. In some embodiments, the second plurality of training molecules includes no more than 1×10¹, no more than 1×10⁸, no more than 1×10⁷, no more than 1×10⁶, no more than 500,000, no more than 100,000, no more than 10,000, or no more than 1000 training molecules. In some embodiments, the second plurality of training molecules consists of from 100 to 1000, from 1000 to 100,000, from 10,000 to 5×10⁶, from 1×10⁶ to 1×10⁷, from 1×10⁷ to 1×10⁸, or from 1×10⁸ to 1×10⁸ training molecules. In some embodiments, the second plurality of training molecules falls within another range starting no lower than 100 training molecules and ending no higher than 1×10¹ training molecules.

In some embodiments, the method further includes, prior to the training, for each respective training molecule in the second plurality of training molecules, obtaining a corresponding three-dimensional pose of the respective training molecule complexed to the target macromolecule or the target macromolecule complex. A corresponding interaction feature vector is determined for the respective training molecule comprising, for each respective interaction feature in the second collection of interaction features, a respective geometric representation of the respective interaction feature in the corresponding three-dimensional pose of the respective training molecule complexed to the target macromolecule or the target macromolecule complex. The corresponding interaction feature vector is transformed using a second reference transformation vector, thereby obtaining, for each respective interaction feature in the second collection of interaction features, the corresponding reference label that indicates whether, or to what degree, the respective interaction feature is causal for a binding selectivity between the respective training molecule and the target macromolecule or the target macromolecule complex.

In some embodiments, the second collection of interaction features includes all or a portion of the first collection of interaction features. In some embodiments, the second collection of interaction features does not share any training molecules in common with the first collection of interaction features. In some implementations, any of the embodiments disclosed herein for a first collection of interaction features are contemplated for use for a second, third, and/or any subsequent collection of interaction features, as will be apparent to one skilled in the art.

In some embodiments, the second reference transformation vector comprises, for each respective interaction feature in the second collection of interaction features, a corresponding binarization threshold for the respective interaction feature. In some embodiments, the transforming applies a dimension reduction to the geometric representation of each respective interaction feature in the corresponding interaction feature vector for the respective training molecule. In some embodiments, the transforming generates, for each respective interaction feature in the corresponding interaction feature vector for the respective training molecule, a corresponding binary value for the respective interaction feature.

### Interaction Features.

Referring to block 230, in some embodiments, a respective interaction feature is selected from the group consisting of: three-dimensional partial charges, three-dimensional pharmacophores, or molecular dynamics residue interaction time.

In some embodiments, a respective interaction feature is selected from the group consisting of hydrophobic interaction, hydrophobic areas, aromatic ring members, hydrogen bond acceptors, hydrogen bond donors, hydrogen bond acceptor in an aromatic ring, negatively charged species, positively charged species, metal coordination, and/or halogen bonds. In some embodiments, a respective interaction feature is a pharmacophore, such as a three-dimensional pharmacophore.

Three-dimensional pharmacophores have been used to capture the nature and three-dimensional arrangement of chemical functionalities in ligands that are relevant for molecular interactions with the macromolecular target. Besides chemical nature and spatial arrangement, three-dimensional pharmacophores can capture feature directionality, such as in the case of hydrogen bonds and aromatic interactions. Additionally, spatial tolerance and weight can be fine-tuned for each pharmacophore feature to adjust its size and importance in the three-dimensional pharmacophore. In order to describe the preferable shape of molecules in the binding site, pharmacophore features are often combined with exclusion volume constraints (also referred to as excluded volume constraints). For instance, an exclusion volume constraint can consist of a set of spheres that represent the protein residues imposing a barrier for binding of potential ligands.

Various tools are available in the art for modeling pharmacophores for ligand-target interactions, including but not limited to FLAP, Pharmer, LigandScout, Catalyst, MOE, PHASE, Pharao, UNITY, and/or Forge. Three-dimensional pharmacophore elucidation methods can be classified as feature-based, substructure pattern-based, or molecular field-based, depending on how the pharmacophore features are derived. Feature-based methods derive pharmacophore features by filtering for geometric descriptors that match the characteristics of molecular interactions. Pattern-based methods, such as those implemented in PHASE, LigandScout, and Catalyst, detect substructures for chemical features in molecules. For example, all hydroxyl groups are defined as hydrogen bond donors and acceptors. In contrast, molecular field-based methods such as FLAP and Forge sample the molecular surface of either ligand or macromolecular target with different chemical probes and calculate interaction energy maps which can be translated into pharmacophore features. An additional distinction between three-dimensional pharmacophore generation methods is based on the type of employed data. This could be a set of active ligands, structural data on the ligand in complex with its macromolecular target, and/or structural data of the macromolecular target alone. Pharmacophores are further described, for example, in Schaller D, Sribar D, Noonan T, et al., “Next generation 3D pharmacophore modeling,” WIRES Comput Mol Sci. 2020; 10(4); Jiang L, Rizzo R C, “Pharmacophore-based similarity scoring for dock,” J Phys Chem B. 2015; 119(3):1083-1102; and Arthur G, Oliver W, Klaus B, et al., “Hierarchical graph representation of pharmacophore models,” Front Mol Biosci. 2020; 7:599059, each of which is hereby incorporated herein by reference in its entirety.

In some embodiments, a respective interaction feature includes one or more corresponding geometric representations and/or one or more attribute values. In some embodiments, the dimensionality and nature of the geometric representations and/or attribute values of interaction features are dependent on the type of interaction feature; that is, a corresponding measurement appropriate for the respective interaction feature, as will be apparent to one skilled in the art. For instance, in some embodiments, a geometric representation of a respective interaction feature is a set of coordinates that indicates the position of the respective interaction feature in three-dimensional space for a respective conformation of the complex formed between a respective molecule and a corresponding target macromolecule or target macromolecule complex. In some embodiments, a geometric representation of a respective interaction feature is a direction vector that indicates the direction or orientation of the respective interaction feature in three-dimensional space for the respective conformation of the complex formed between the respective molecule and the corresponding target macromolecule or target macromolecule complex.

As another example, in some embodiments, an attribute value for a partial charge is a non-integer charge value when measured in elementary charge units; in yet another example, in some implementations, an attribute value for an aromatic ring pharmacophore includes a radius r of the aromatic ring.

Alternatively or additionally, in some embodiments, an attribute value for a respective interaction feature is a similarity score that measures a difference or a distance between the respective interaction feature in in a candidate conformation of a complex formed between the respective molecule and the corresponding target macromolecule or target macromolecule complex and a corresponding interaction feature in a reference conformation.

Alternatively or additionally, in some embodiments, an attribute value for a respective interaction feature is an indication of a presence or absence of the respective interaction feature at a corresponding position in a respective conformation of a complex formed between a respective molecule and a corresponding target macromolecule or target macromolecule complex. In some embodiments, a corresponding geometric representation and/or a corresponding attribute value for a respective interaction feature is represented in a multi-dimensional space; for instance, in some embodiments, an attribute value for a hydrophobic interaction feature is represented as (1, 0, 0).

Interaction features are further described, for example, in Jiang L, Rizzo R C, “Pharmacophore-based similarity scoring for dock,” J Phys Chem B. 2015; 119(3):1083-1102; and Arthur G, Oliver W, Klaus B, et al., “Hierarchical graph representation of pharmacophore models,” Front Mol Biosci. 2020; 7:599059, each of which is hereby incorporated herein by reference in its entirety.

In some embodiments, one or more dimension reduction techniques are applied to one or more geometric representations and/or one or more attribute values for a respective interaction feature.

In some embodiments, a dimension reduction reduces the dimensionality of a respective interaction feature from a first number of dimensions to a second number of dimensions. In some implementations, the starting number of dimensions varies between interaction features (e.g., a first interaction feature in a plurality of interaction features has the same or different number of starting dimensions as a second interaction feature in the plurality of interaction features). In some embodiments, the second number of dimensions after dimension reduction is the same or different for each interaction feature in a plurality of interaction features. For example, in some implementations, each respective interaction feature in a plurality of interaction features has a dimensionality of 1 after transformation.

In some embodiments, the dimension reduction is a principal components algorithm, a random projection algorithm, an independent component analysis algorithm, a feature selection method, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi:10.5772/16863. ISBN 978-953-307-996-7; and Lakshmi et al., 2016, “2016 IEEE 6th International Conference on Advanced Computing (IACC),” pp. 31-34. doi:10.1109/IACC.2016.16, ISBN 978-1-4673-8286-1, each of which is hereby incorporated by reference.

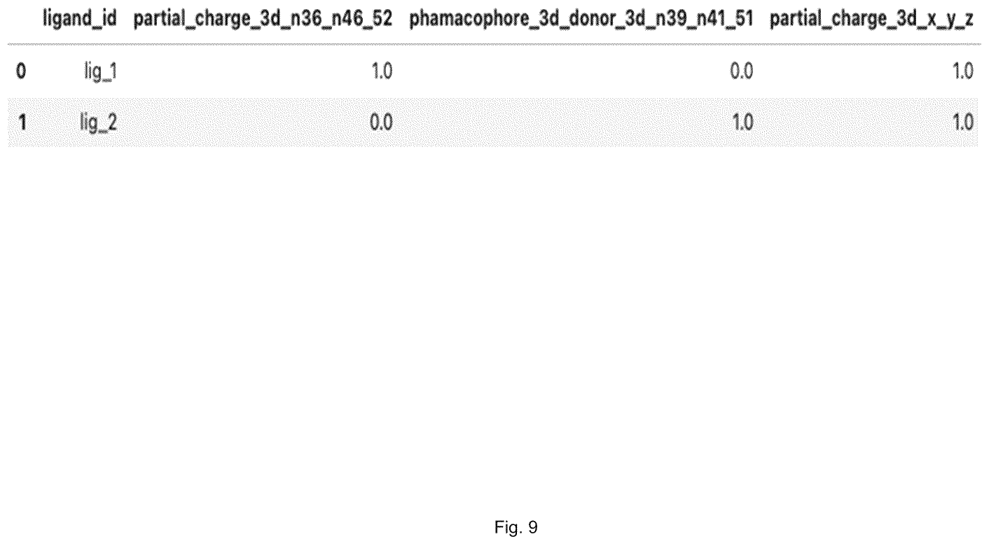

In some implementations, a geometric representation and/or an attribute value for a respective interaction feature is represented in scalar or binary values. In some implementations, upon application of a transformation to a respective interaction feature, the geometric representation and/or attribute value is further transformed from scalar values to binary values (e.g., 0 or 1). An example of an interaction feature vector for a corresponding candidate molecule, where the geometric representations and/or attribute values for each interaction feature in the interaction feature vector is binarized to 0s and 1s, is illustrated in FIG. 9.

In some implementations, any of the embodiments disclosed herein for a first plurality of interaction features is similarly contemplated for use with a second, third, fourth, fifth, or any subsequent plurality of interaction features, or any substitutions, modifications, additions, deletions, and/or combinations thereof, as will be apparent to one skilled in the art. For instance, in some embodiments, the first plurality of interaction features is obtained using the same method as the third and/or fifth plurality of interaction features (e.g., using a causal binding hypothesis). In some embodiments, the second plurality of interaction features is obtained using the same method as the fourth plurality of interaction features (e.g., using a causal selectivity hypothesis). In some embodiments, any one plurality of interaction features disclosed herein is obtained using a different method as any other plurality of interaction features disclosed herein (e.g., using a causal binding hypothesis, a causal selectivity hypothesis, a first model, a second model, a third model, or any subsequent model, or any components or ensembles thereof). Moreover, in some embodiments, any one plurality of interaction features disclosed herein includes all, none, or a portion of any other plurality of interaction features disclosed herein.

### Additional Embodiments for Machine Learning Models.

Referring to block 232, in some embodiments, the performing B) further comprises, for each respective candidate molecule in the plurality of candidate molecules, responsive to inputting a corresponding representation of a chemical structure of the respective candidate molecule into a third model, retrieving, as output from the third model, a corresponding measure of activity for the respective candidate molecule; and using the corresponding measure of activity to obtain the corresponding second score for the interaction between the respective candidate molecule and the target macromolecule or the target macromolecule complex.

Referring to block 234, in some embodiments, the corresponding measure of activity is an ADME (e.g., absorption, distribution, metabolism, and excretion) score. In some embodiments, the third model accepts, as input, for each respective candidate molecule in the plurality of candidate molecules, a corresponding molecular fingerprint and/or a two-dimensional molecular graph. In some embodiments, the corresponding measure of activity is an ADMET (absorption, distribution, metabolism, excretion, and toxicity) score.

Typically, drug development involves assessment of absorption, distribution, metabolism, and excretion (ADME) and/or toxicity (ADMET) to determine the effectiveness of a candidate molecule as a drug. Such effectiveness is measured, in some implementations, as the ability of a candidate molecule to reach its target in the subject in sufficient concentration, maintain bioactivity for long enough to achieve a target effect, and cause minimal toxicity. In some implementations, ADME or ADMET properties are determined using any one or more of a variety of techniques, including but not limited to substructure searches, molecular fingerprint methods, support vector machine (SVM) or Bayesian techniques, and/or deep neural networks. Various tools for predicting ADME or ADMET properties are known in the art and provide indications of a candidate molecule's physicochemical properties, pharmacokinetics, drug-likeness and/or medicinal chemistry friendliness, among others. Examples of such models include, but are not limited to, SwissADME, pk-CSN, admetSAR, iLOGP, BOILED-Egg, and/or Bioavailability Radar.

Any number of ADME or ADMET models are contemplated for use in the present disclosure. For instance, available tools for predicting ADME or ADMET properties include those that focus on all or less than all ADME or ADMET properties. Accordingly, in some implementations, a plurality of ADME or ADMET models are used to determine a broad range of target properties, where each respective ADME or ADMET model outputs a corresponding measure of activity for the respective candidate molecule that corresponds to one or more respective ADME or ADMET properties in a plurality of ADME or ADMET properties. ADME and ADMET models are further described, for example, in Daina A, Michielin O, Zoete V, “SwissADME: a free web tool to evaluate pharmacokinetics, drug-likeness and medicinal chemistry friendliness of small molecules,” Sci Rep. 2017; 7(1):42717, which is hereby incorporated by reference in its entirety.

Thus, in some embodiments, the third model comprises at least 1, at least 2, at least 3, at least 5, at least 10, or at least 20 component models. In some embodiments, the third model includes no more than 20, no more than 15, no more than 10, or no more than 5 component models. In some embodiments, the third model consists of from 1 to 5, from 2 to 10, from 5 to 18, or from 10 to 20 component models. In some embodiments, the third model includes a plurality of component models that falls within another range starting no lower than 1 model and ending no higher than 20 models. Additional models beyond the third model are also contemplated for use in the present disclosure.

In some embodiments, the corresponding measure of activity includes a corresponding at least 1, at least 2, at least 3, at least 5, at least 10, or at least 20 measures of activity. In some embodiments, the corresponding measure of activity includes no more than 20, no more than 15, no more than 10, or no more than 5 measures of activity. In some embodiments, the corresponding measure of activity consists of from 1 to 5, from 2 to 10, from 5 to 18, or from 10 to 20 measures of activity. In some embodiments, the corresponding measure of activity falls within another range starting no lower than 1 and ending no higher than 20 measures of activity.

In some embodiments, the measure (or measures) of activity are further used to obtain the corresponding second score for the interaction between the respective candidate molecule and the target macromolecule or the target macromolecule complex.

In some embodiments, the second score is aggregated based on the respective outputs of the first model, the second model, the third model, and/or any subsequent models, or any components or ensembles thereof, as will be apparent to one skilled in the art. In some embodiments, the second score is a measure of central tendency (e.g., mean, median, mode, weighted mean, weighted median, and/or weighted mode) based on the respective outputs of the first model, the second model, the third model, and/or any subsequent models. For example, as shown in FIG. 8B, in an embodiment, the second score is a normalized score generated based on the output from the first model (e.g., binding affinity score), the output from the second model (e.g., binding selectivity score), and the output from the third model (e.g., ADME score).

In some embodiments, the first plurality of parameters comprises at least 10,000, at least 100,000, or at least 1×10⁶ parameters. In some embodiments, the second plurality of parameters comprises at least 10,000, at least 100,000, or at least 1×10⁶ parameters.

In particular, in some embodiments, the first and/or second plurality of parameters includes at least 10, at least 100, at least 1000, at least 10,000, at least 100,000, at least 1×10⁶, at least 1×10⁷, or more parameters. In some embodiments, the first and/or second plurality of parameters includes no more than 1×10⁸, no more than 1×10⁷, no more than 1×10⁶, no more than 100,000, no more than 10,000, no more than 1000, or no more than 100 parameters. In some embodiments, the first and/or second plurality of parameters consists of from 10 to 1000, from 100 to 100,000, from 10,000 to 1×10⁷, or from 1×10⁶ to 1×10⁸ parameters. In some embodiments, the first and/or second plurality of parameters falls within another range starting no lower than 10 parameters and ending no higher than 1×10⁸ parameters.

### Causal Binding Hypothesis Generation.

In some embodiments, a causal binding hypothesis is used to generate a plurality of interaction features, geometric representations, attribute values, and/or a representation thereof, as described above. Alternatively or additionally, in some embodiments, a causal binding hypothesis is used to generate a first reference transformation vector, as described above.

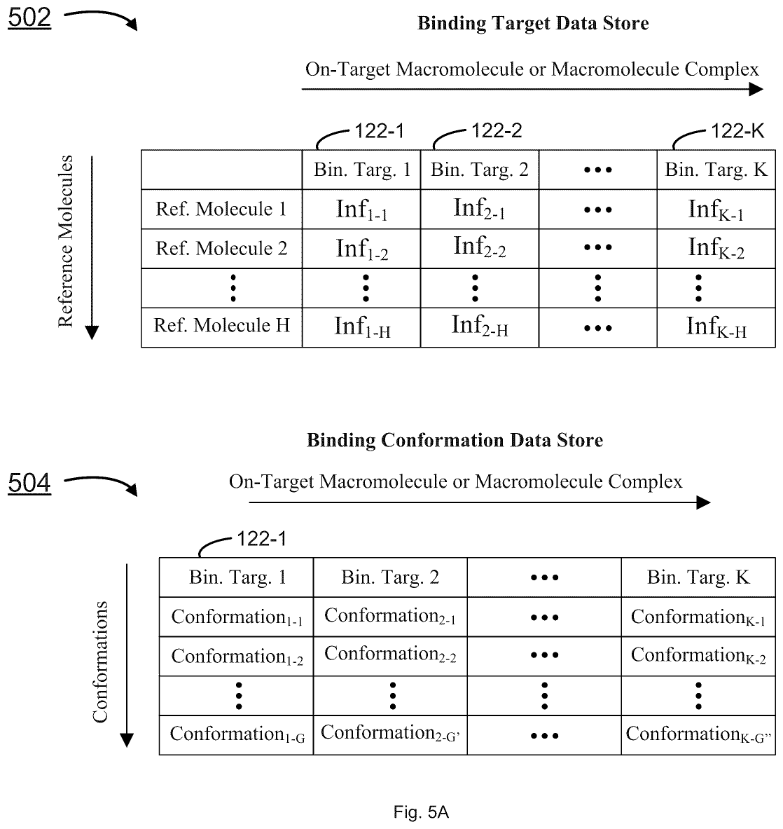

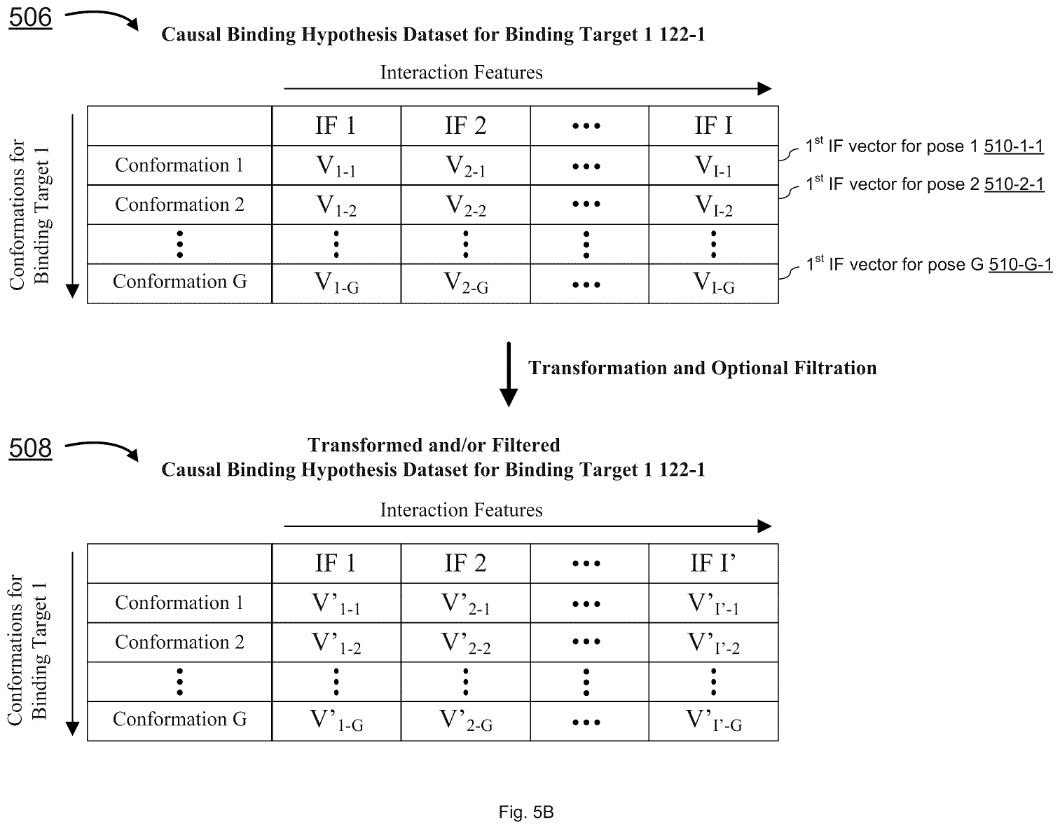

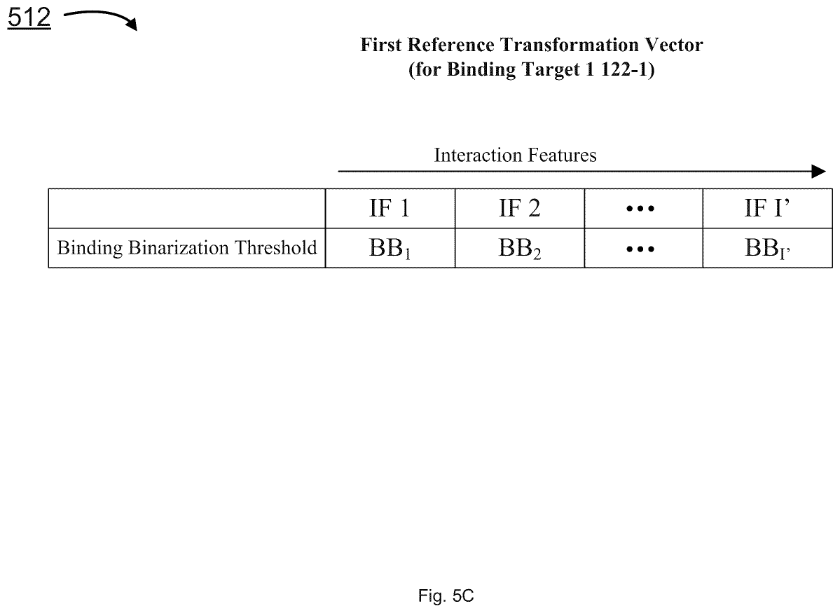

An example causal binding hypothesis generation process is shown as block 302 within pipeline 300 for characterizing interactions between candidate molecules and target macromolecules, depicted in FIG. 3. Causal binding hypothesis generation process 302 is further depicted as a workflow in FIG. 4A, with reference to data structures 502, 504, 506, 508, 510, and 512 in FIGS. 5A-C.

In some embodiments, the causal binding hypothesis generation procedure includes, for each respective reference molecule in a first plurality of reference molecules, (i) obtaining one or more corresponding three-dimensional poses of the respective reference molecule complexed to the target macromolecule or the target macromolecule complex, where the corresponding three-dimensional pose comprises a respective measure of on-target binding energy, thereby obtaining a plurality of three-dimensional poses.

In some embodiments, the first plurality of reference molecules includes ligands for binding to the target macromolecule or target macromolecule complex.

In some embodiments, the first plurality of reference molecules includes at least 100, at least 500, at least 1000, at least 5000, at least 10,000, at least 100,000, at least 1×10⁶, at least 5×10⁶, at least 1×10⁷, at least 1×10⁸, or at least 5×10⁸ reference molecules. In some embodiments, the first plurality of reference molecules includes no more than 1×10¹, no more than 1×10⁸, no more than 1×10⁷, no more than 1×10⁶, no more than 500,000, no more than 100,000, no more than 10,000, or no more than 1000 reference molecules. In some embodiments, the first plurality of reference molecules consists of from 100 to 1000, from 1000 to 100,000, from 10,000 to 5×10⁶, from 1×10⁶ to 1×10⁷, from 1×10⁷ to 1×10⁸, or from 1×10⁸ to 1×10⁸ reference molecules. In some embodiments, the first plurality of reference molecules falls within another range starting no lower than 100 reference molecules and ending no higher than 1×10¹ reference molecules.

In some embodiments, for a respective reference molecule in the first plurality of reference molecules, the one or more corresponding three-dimensional poses of the respective reference molecule complexed to the target macromolecule or the target macromolecule complex includes at least 1, at least 2, at least 3, at least 4, at least 5, at least 10, at least 50, at least 100, or at least 1000 three-dimensional poses. In some embodiments, the one or more three-dimensional poses comprises no more than 5000, no more than 1000, no more than 100, no more than 50, no more than 10, no more than 5, or no more than 2 three-dimensional poses. In some embodiments, the one or more three-dimensional poses consists of from 1 to 10, from 5 to 100, from 50 to 1000, or from 100 to 5000 three-dimensional poses. In some embodiments, the one or more three-dimensional poses falls within another range starting no lower than 1 three-dimensional pose and ending no higher than 1000 three-dimensional poses.

In some embodiments, the plurality of three-dimensional poses for the target macromolecule or macromolecule complex includes at least 100, at least 500, at least 1000, at least 5000, at least 10,000, at least 100,000, at least 1×10⁶, at least 5×10⁶, at least 1×10⁷, at least 1×10⁸, or at least 5×10⁸ three-dimensional poses. In some embodiments, the plurality of three-dimensional poses for the target macromolecule or macromolecule complex includes no more than 1×10⁹, no more than 1×10⁸, no more than 1×10⁷, no more than 1×10⁶, no more than 500,000, no more than 100,000, no more than 10,000, or no more than 1000 three-dimensional poses. In some embodiments, the plurality of three-dimensional poses for the target macromolecule or macromolecule complex consists of from 100 to 1000, from 1000 to 100,000, from 10,000 to 5×10⁶, from 1×10⁶ to 1×10⁷, from 1×10⁷ to 1×10⁸, or from 1×10⁸ to 1×10⁸ three-dimensional poses. In some embodiments, the plurality of three-dimensional poses for the target macromolecule or macromolecule complex falls within another range starting no lower than 100 three-dimensional poses and ending no higher than 1×10⁹ three-dimensional poses.

In some embodiments, a first respective target in a plurality of targets has the same or different number of three-dimensional poses in a corresponding plurality of three-dimensional poses as a second respective target in the plurality of targets. Moreover, in some embodiments, for a respective target macromolecule or macromolecule complex, a first respective reference molecule in the first plurality of reference molecules has the same or different number of three-dimensional poses in the corresponding one or more three-dimensional poses as a second respective reference molecule in the first plurality of reference molecules.

In some embodiments, the target macromolecule or macromolecule complex is a polymer with an active site, and each of the poses is obtained by docking the reference molecule into the active site of the target macromolecule or macromolecule complex. In some embodiments, the reference molecule is docked onto the target macromolecule or macromolecule complex a plurality of times to form a plurality of poses. In some embodiments, the reference molecule is docked onto the target macromolecule or macromolecule complex at least 2, at least 3, at least 4, at least 5, at least 10, at least 50, at least 100, or at least 1000 times. Each such docking represents a different pose of the reference molecule docked onto the target macromolecule or macromolecule complex. In some embodiments, the target macromolecule or macromolecule complex is a polymer with an active site and the reference molecule is docked into the active site in each of plurality of different ways, each such way representing a different pose. In some embodiments, the target macromolecule or macromolecule complex comprises a plurality of active sites and the reference molecule is docked into one or more of the active sites in each of plurality of different ways, each such way representing a different pose. In some such embodiments, separate studies are individually also conducted on one or more of the other active sites of the target macromolecule or macromolecule complex using the systems and methods of the present disclosure.

In some embodiments, for a respective reference molecule in the first plurality of reference molecules, the one or more corresponding three-dimensional poses are obtained using any of the methods disclosed herein (see, e.g., the section entitled “Definitions: Pose,” above). In some embodiments, for a respective reference molecule in the first plurality of reference molecules, the one or more corresponding three-dimensional poses are obtained from any of a variety of sources including, but not limited to, structure ensembles generated by solution NMR, co-complexes as interpreted from X-ray crystallography, neutron diffraction, cryo-electron microscopy, sampling from computational simulations, homology modeling, rotamer library sampling, or any combination thereof.

In some embodiments, for a respective reference molecule in the first plurality of reference molecules, the one or more corresponding three-dimensional poses are obtained using DiffDock. DiffDock is a diffusion generative model (DGM) developed over the space of ligand poses for molecular docking. The diffusion process is defined over the degrees of freedom involved in docking, including the position of the ligand relative to the protein (locating the binding pocket), its orientation in the pocket, and the torsion angles describing its conformation. DiffDock samples poses by running the learned (reverse) diffusion process, which iteratively transforms an uninformed, noisy prior distribution over ligand poses into the learned model distribution. See, for example, Corso et al., “DiffDock: Diffusion steps, twists, and turns for molecular docking,” ICLR 2023, available on the Internet at arXiv:2210.01776v2, which is hereby incorporated herein by reference in its entirety.

In some embodiments, each respective pose comprises a respective measure of on-target binding energy (e.g., a known label) that indicates a binding affinity for the respective conformation of the reference molecule when complexed to the target. In some embodiments, the respective measure of on-target binding energy is Gibbs free energy.

In some embodiments, the causal binding hypothesis generation procedure further includes, for each three-dimensional pose in the plurality of three-dimensional poses, (ii) determining a first interaction feature vector for the respective pose comprising, for each respective interaction feature in a first collection of interaction features, one or more respective geometric representations and/or one or more respective attribute values for the respective interaction feature in the corresponding three-dimensional pose of the respective reference molecule complexed to the target macromolecule or the target macromolecule complex, thereby obtaining a plurality of first interaction feature vectors.

In some embodiments, the causal binding hypothesis generation procedure further includes performing one or more filtering steps and/or one or more transformations to each respective interaction feature vector in the plurality of first interaction feature vectors.

In some implementations, the one or more filtering steps and/or one or more transformations further includes removing, from the first collection of interaction features, each respective interaction feature that has a geometric representation and/or an attribute value that fails to satisfy a first filtering criterion in each respective pose in the plurality of poses for the target macromolecule or macromolecule complex. In some implementations, a respective interaction feature fails to satisfy the first filtering criterion when the respective interaction feature has a value of zero across all poses for the target macromolecule or macromolecule complex.

In some implementations, the one or more filtering steps and/or one or more transformations further includes removing, from the first collection of interaction features, each respective interaction feature that has a geometric representation and/or an attribute value that fails to satisfy a second filtering criterion across the plurality of poses for the target macromolecule or macromolecule complex. In some implementations, a respective interaction feature fails to satisfy the second filtering criterion when the respective interaction feature has a measure of dispersion that is below a threshold dispersion across all poses for the target macromolecule or macromolecule complex. In some embodiments, the measure of dispersion is a variance, standard deviation, and/or standard error.

In some implementations, the one or more filtering steps and/or one or more transformations further includes inputting each corresponding interaction feature vector as input to a segmentation algorithm, thereby obtaining, for each respective interaction feature in the first collection of interaction features, a corresponding binary value for the respective interaction feature in the corresponding three-dimensional pose of the respective reference molecule complexed to the target macromolecule or the target macromolecule complex.

In the context of images, segmentation refers to the process of subdividing a digital image into multiple regions or objects. The goal of segmentation is to simplify or change the representation of an image into a form in which patterns and distinct regions are more easily differentiated. Segmentation algorithms include region-based, boundary-based, thresholding, or hybrid methods. Image thresholding is an efficient technique for image segmentation applications and for pattern recognition, in which a particular threshold is selected. Various approaches for thresholding further include global and local thresholding. Global thresholding techniques segment the entire image using a single global threshold based on gray level values. Local thresholding techniques segment the image into smaller sub-images for which thresholds are calculated depending on local properties of each respective point in the image, as well as its respective position and gray level values. Various methods for thresholding are known in the art, including but not limited to artificial bee colony (ABC), locust swarms (LS), cuckoo search, particle swarm optimization and/or metaheuristics. Metaheuristic methods involve a stochastic process, executing random operations which lead to slow execution. Entropic thresholding is used to minimize the cross entropy (minimum cross-entropy thresholding (MCET)) between the original and the segmented images through selecting an optimum threshold between two probabilistic distributions. Methods for segmentation and/or thresholding are further described in Al-Ajlan and El-Zaart, “Image Segmentation Using Minimum Cross-Entropy Thresholding,” Proceedings of the 2009 IEEE International Conference on Systems, Man, and Cybernetics, doi: 10.1109/ICSMC.2009.5346619; and Rawas, S. and El-Zaart, A. (2020), “Precise and parallel segmentation model (PPSM) via MCET using hybrid distributions,” Applied Computing and Informatics, doi: 10.1108/ACI-11-2020-0123, each of which is hereby incorporated herein by reference in its entirety.

Segmentation and/or thresholding techniques can be similarly applied to datasets, in which pixels or points of an image are represented as feature values (e.g., coordinate sets and/or attribute values of a respective interaction feature for a respective pose, as shown, for instance, in FIG. 5B. Moreover, an illustrative example of an interaction feature vector that has undergone thresholding is illustrated in FIG. 9.

In some embodiments, the thresholding algorithm is binary cross-entropy. As described above, in some implementations, a thresholding algorithm seeks to determine a threshold between two or more distributions in a dataset (such as an image, which can also be represented as values in a matrix). In this case, the thresholding algorithm seeks to determine a threshold between distributions of continuous variables represented in the target dataset as values for three-dimensional coordinates. Binary cross-entropy thresholding utilizes an information gain approach in that entropy is related to the uncertainty that exists when sampling from one or more distributions (e.g., higher entropy indicating greater uncertainty). Thus, the approach determines a threshold that minimizes a cross-entropy calculated for the original dataset (e.g., in three dimensions) and the transformed dataset (e.g., in one dimension). The cross-entropy to be minimized can be represented by the equation H(P,Q)=−P(x)log(Q(x)) summed over X, for a first distribution P and a second distribution Q.

In some implementations, the one or more filtering steps and/or one or more transformations further includes performing a debias strategy comprising propensity score matching (PSM). PSM is a statistical matching technique that attempts to estimate the effect of a treatment, policy, or other intervention by accounting for the covariates that predict receiving the treatment. PSM thus reduces bias due to confounding variables that could be found in an estimate of the treatment effect obtained from simply comparing outcomes among units that received the treatment versus those that did not. For instance, in some implementations, the debias strategy is a fingerprint debias strategy that seeks to remove interaction features that are included due to structural bias. In some implementations, a particular structural characteristic acts as a confounding variable that results in interaction features coincident with this structural characteristic to appear causal for binding, when in fact the interaction features in isolation do not have such effect. In some implementations, comparing interaction feature profiles across active and inactive reference molecules having similar chemical structures (e.g., via Tanimoto similarity), allows for the identification of causal interaction features while accounting for structure-based confounders.

For each respective interaction feature in the first collection of interaction features, the respective interaction feature is assigned as a “treatment” and all other interaction features, other than the respective interaction feature, is assigned as a confounder or covariate. In some embodiments, a treatment refers to a variable that is being tested for its effect on an outcome. In an example embodiment, a treatment includes a given partial charge that affects delta G, given the other partial charges as confounders. In some embodiments, a confounder refers to a variable that influences both the dependent and independent variable, causing a spurious association. In the example implementation, confounders include other partial charges in a hypothesis other than the partial charge representing the treatment.

For each respective reference molecule in the first plurality of reference molecules, for each respective interaction feature I in the first collection of interaction features IF, a propensity score is determined using logistic regression P=−(Î/ÎF). For each respective reference molecule in the first plurality of reference molecules, (i) the plurality of propensity scores for the corresponding plurality of interaction features in the respective reference molecule is matched with (ii) a corresponding plurality of matched propensity scores for a corresponding plurality of interaction features of a respective matching reference molecule, thus obtaining a plurality of matched pairs of reference molecules. In some embodiments, the matching comprises nearest neighbor matching. In some embodiments, the matching matches each respective reference molecule in the first plurality of reference molecules with a unique matched reference molecule in the first plurality of reference molecules (e.g., each reference molecule is matched with only one other reference molecule).

In some embodiments, other methods for determining similarity between reference molecules are contemplated. For instance, non-limiting example methods for classifying reference molecules as active or inactive include naïve Bayes methods, Tanimoto similarity, Dice similarity, Cosine similarity, Substructure similarity, Superstructure similarity, distance-based similarity (e.g., derived from the Manhattan, Euclidean and Soergel distances), support vector machine, graph convolutional neural network fingerprint, and/or random matrix discriminant. See, for example, Lee A A, Yang Q, Bassyouni A, et al., “Ligand biological activity predicted by cleaning positive and negative chemical correlations,” Proc Natl Acad Sci USA. 2019; 116(9):3373-3378; and Bajusz D, Ricz A, Héberger K, “Why is Tanimoto index an appropriate choice for fingerprint-based similarity calculations?” Journal of Cheminformatics. 2015; 7(1):20, each of which is hereby incorporated herein by reference in its entirety.

In some embodiments, the matching matches each respective reference molecule in a first subset of reference molecules in the plurality of reference molecules with a corresponding matching reference molecule in a second subset of reference molecules. In some embodiments, the first subset of reference molecules is active molecules, and the second subset of reference molecules is inactive molecules.

In some embodiments, a respective reference molecule is classified as active or inactive according to their biological activity. For instance, in an illustrative implementation, a reference molecule is considered active against a given target if its Ki, Kd, IC50, or EC50 is 1 pM or less and inactive otherwise. In some embodiments, an activity of a respective reference molecule is determined using experimental data. Alternatively or additionally, in some embodiments, an activity of a respective reference molecule is determined using a compound database. Example databases contemplated for use in the present disclosure include, but are not limited to, ChEMBL, Pfizer, PDBeChem, Protein Data Bank (PDB), KEGG Compound Database, Natural Ligand DB, PubChem, and/or DrugBank. In some embodiments, a respective reference molecule is classified as active or inactive if it is chemically similar to one or more known active molecules and/or known inactive molecules. For example, in some embodiments, a respective reference molecule is classified as active or inactive based on a presence or absence of one or more chemical motifs that are associated with one or more known active molecules or one or more known inactive molecules. Active and inactive reference molecules are further described, for example, in Lee A A, Yang Q, Bassyouni A, et al., “Ligand biological activity predicted by cleaning positive and negative chemical correlations,” Proc Natl Acad Sci USA. 2019; 116(9):3373-3378; and Kim S, “Getting the most out of PubChem for virtual screening,” Expert Opin Drug Discov. 2016; 11(9):843-855, each of which is hereby incorporated herein by reference in its entirety.

In some embodiments, the propensity score matching strategy is performed using the molecular fingerprint of each respective reference molecule in the first plurality of reference molecules, including but not limited to Morgan fingerprint, extended-connectivity fingerprint ECFP4 in 1024-dimensions, extended-connectivity fingerprint ECFP4 in 2048-dimensions, substructure fingerprint, atom-pair (AP) fingerprint, MinHashed fingerprint MHFP6 in 1024-dimensions, MinHashed fingerprint MHFP6 in 2048-dimensions, MinHashed atom-pair fingerprint up to four bonds (MAP4), MXFP (macromolecule extended atom-pair fingerprint, 217-dimensions atom-pair fingerprint), Topological Torsion (TT) fingerprint, MACCS fingerprint, SEFP4, LCFP4 and FCFP4/6 fingerprints, Structural Protein-Ligand Interaction Fingerprint (SPLIF), structural interaction fingerprint (SIFt), atom-pairs-based interaction fragment (APIFs), and/or ECFP0 fingerprint. Molecular fingerprints are further disclosed, for example, in Capecchi A, Probst D, Reymond J L, “One molecular fingerprint to rule them all: drugs, biomolecules, and the metabolome,” J Cheminform. 2020; 12:43, which is hereby incorporated herein by reference in its entirety.

Accordingly, in some embodiments, the one or more filtering steps and/or one or more transformations further includes performing a debias strategy comprising propensity score matching (PSM), including: obtaining, for each reference molecule in the first plurality of reference molecules, a corresponding molecular fingerprint (e.g., Morgan Fingerprint). The first plurality of reference molecules is subdivided into an “actives” subset and an “inactives” subset. For each active reference molecule in the “actives” subset, a closest matching inactive reference molecule in the “inactives” subset is determined based on a similarity measure between the molecular fingerprint of the respective active reference molecule and the molecular fingerprint of each inactive reference molecule in the “inactives” subset (e.g., via Tanimoto similarity), thus obtaining a set of reference molecule pairs. For each interaction feature in the first collection of interaction features, a tally of the difference in entry values corresponding to the respective interaction feature between each reference molecule pair in the set of reference molecule pairs is obtained. Interaction features that have a negative tally are removed from the first collection of interaction features.

In some embodiments, the causal binding hypothesis generation procedure further includes (iii) responsive to inputting each respective first interaction feature vector in the plurality of first interaction feature vectors into a causal inference model, retrieving, as output from the causal inference model, for each respective interaction feature in the first collection of interaction features, a corresponding feature score for the respective interaction feature; and (iv) removing, from the respective first interaction feature vector, each respective interaction feature having a corresponding feature score that fails to satisfy a threshold feature criterion, thereby obtaining a first plurality of reference interaction features. In some embodiments, one or more interaction features are not removed from the respective first interaction feature vector.

In some embodiments, the causal inference model is a double machine learning causal forest (DMLCF), and the corresponding feature score is determined as:

$$Y = {β}_{0} + D * {β}_{D} + θ X + e ,$$

where D is a first interaction feature in the first collection of interaction features, X is each respective interaction feature, other than the first interaction feature, in the first collection of interaction features, Y is the respective measure of on-target binding energy for the three-dimensional pose of the respective candidate molecule complexed to the target macromolecule or the target macromolecule complex, and the corresponding feature score is an average treatment effect.

In an example implementation of DMLCF, 3 regressions are used to estimate the coefficient RD in the regression equation: Y=β0+D*βD+θX+e. Regression 1 plots Y on X (e.g., how well confounders relate to the outcome). In the resulting plot, each data point in a plurality of data points represents a different pose in a plurality of poses, and X represents a vector of “confounder” interaction feature values corresponding to the pose, such that the regression is a multivariable regression. The residuals (e.g., error) for each data point to the regression line are stored as ω. Regression 2 plots D on X (e.g., how well confounders relate to the treatment). Residuals are stored as ε. Regression 3 plots ε on ω (e.g., residuals-on-residuals). This regression isolates the variation in D that is orthogonal to X, thus providing an indication of causality in the treatment without confounder influence. The coefficient for ω (e.g., ε=aω+b) approximates βD, or the ATE.

As described above, the causal inference analysis seeks to remove interaction features that may be included due to association with confounders. The aim is to identify interaction features that have an independent, actual causal effect on binding, rather than those that are merely affected by the presence of other interaction features and are not directly causal. In some embodiments, average treatment effect (ATE) refers to a measure used to compare treatments (or interventions) in randomized experiments. The ATE measures the difference in mean outcomes between units assigned to the treatment and units assigned to the control. In yet another example implementation, the ATE measures the effect of a treatment (e.g., a single partial charge) on delta G (e.g., where the given partial charge when activated drops delta G by −1.0).

In some embodiments, the corresponding feature score fails to satisfy a threshold feature criterion when the corresponding feature score is greater than zero (e.g., raw ATEs greater than zero). For example, and without being limited to any one theory of operation, in some implementations, because a negative value of G indicates a stronger binding affinity, targeting interaction features with negative ATEs for retention can be used to select for interaction features that are causal for binding. ATEs are further described, for example, in McConnell K J, Lindner S, “Estimating treatment effects with machine learning,” Health Serv Res. 2019; 54(6):1273-1282, which is hereby incorporated herein by reference in its entirety.

In another example implementation, the feature score is further determined based on the following equations:

$$for each I in IF \\ Y = g ( I , \hat{IF} ) + U , E [ U \vert \hat{IF} , I ] = 0 \\ D = m ( \hat{IF} ) + V , E [ V \vert \hat{IF} ] = 0 \\ a ( I , Z ) = \frac{I}{m ( \hat{IF} )} - \frac{1 - I}{1 - m ( \hat{IF} )} \\ m ( Z ) = g ( 1 , \hat{IF} ) - g ( 0 , \hat{IF} ) \\ T = E [ g ( 1 , \hat{IF} ) - g ( 0 , \hat{IF} ) ] \\ ATE = T - m ( \hat{IF} ) - a ( I , \hat{IF} ) ( Y - g ( I , \hat{IF} ) )$$

where IF represents all of the interaction features, I is the individual interaction feature being used to test treatment, both g and m are random forest or XGBoost models, and Y is Gibbs free energy.

Alternatively or additionally, in some embodiments, the causal inference model comprises propensity score matching (PSM). Moreover, any suitable embodiments for PSM as disclosed above are contemplated for use in causal inference. For instance, in some embodiments, the PSM includes, for each respective interaction feature I assigned as a treatment D in the first collection of interaction features IF, for each respective reference molecule in the first plurality of reference molecules, determining a respective propensity score for the respective interaction feature for the respective reference molecule using a logistic regression P=σ(Î/ÎF). For each respective interaction feature I in the first collection of interaction features IF, for each respective reference molecule in the first plurality of reference molecules, (i) the corresponding propensity score for the respective interaction feature in the respective reference molecule is matched with (ii) a corresponding propensity score for the respective interaction feature of a respective matching reference molecule in the first plurality of reference molecules, thus obtaining one or more subsets of matched reference molecules. In some embodiments, the matching comprises nearest neighbor matching. In some embodiments, the matching matches each respective reference molecule with one or more matched reference molecules in the first plurality of reference molecules. In some embodiments, the matching matches each respective reference molecule with a unique matched reference molecule in the first plurality of reference molecules (e.g., each reference molecule is matched with only one other reference molecule).

In some embodiments, the matching matches each respective reference molecule in a first subset of reference molecules in the plurality of reference molecules with a corresponding matching reference molecule in a second subset of reference molecules. In some embodiments, the first subset of reference molecules is active molecules, and the second subset of reference molecules is inactive molecules.

In some embodiments, the performing causal inference via PSM further includes, for each respective interaction feature I in the first collection of interaction features IF, determining a respective treatment effect based on a difference between, for each respective subset of matched reference molecules in the one or more subsets of matched reference molecules, (i) a measure of interaction for a first reference molecule and (ii) a measure of interaction for a second reference molecule. In some embodiments, the treatment effect is an average treatment effect, each respective subset of matched reference molecules is a matched pair of reference molecules in a plurality of matched pairs, each respective matched pair of reference molecules comprises a first reference molecule selected from a first subset of active molecules and a second reference molecule selected from a second subset of inactive molecules, the measure of interaction is Gibbs free energy, and the determining the respective treatment effect comprises (i) for each respective matched pair of reference molecules, determining the difference in Gibbs free energy between the first reference molecule and the second reference molecule (delta G) and (ii) averaging the difference in Gibbs free energy over all of the matched pair of reference molecules in the plurality of matched pairs.

In some embodiments, the causal binding hypothesis generation procedure further includes performing one or more validation steps.

In some implementations, the one or more validation steps further includes performing a placebo refutation process including repeating the (iii) causal inference model (e.g., using double machine learning causal forest (DMLCF) and/or propensity score matching (PSM)), where for each respective first interaction feature vector in the plurality of first interaction feature vectors, the treatment is replaced with a random variable, thereby obtaining one or more validation ATEs for a corresponding one or more interaction features in the first collection of interaction features. Alternatively or additionally, in some implementations, the one or more validation steps further includes performing a bootstrap refutation process including repeating the (iii) causal inference model (e.g., using double machine learning causal forest (DMLCF) and/or propensity score matching (PSM)) for a random subset of poses in the plurality of poses, thereby obtaining one or more validation ATEs for a corresponding one or more interaction features in the first collection of interaction features.

In some embodiments, the one or more validation steps further includes, for each respective interaction feature in the first collection of interaction features, comparing the ATE from the (iii) causal inference model with the validation ATE, and, when a difference between the ATE and the validation ATE is observed, applying a penalty to the ATE to obtain an updated ATE for the respective interaction feature. In some embodiments, the difference is a statistically significant difference. In some embodiments, the penalty replaces the ATE with the validation ATE. In some such embodiments, the one or more validation steps further includes removing each respective interaction feature in the first collection of interaction features that has an updated ATE greater than zero.

In some embodiments, the first plurality of reference interaction features is further used to generate the first reference transformation vector that includes, for each respective reference interaction feature in the first plurality of reference interaction features (e.g., the filtered and/or transformed first collection of interaction features), a corresponding binarization threshold for the respective reference interaction feature.

### Causal Selectivity Hypothesis Generation.

In some embodiments, a causal selectivity hypothesis is used to generate a plurality of interaction features, geometric representations, attribute values, and/or a representation thereof, as described above. Alternatively or additionally, in some embodiments, a causal selectivity hypothesis is used to generate a second reference transformation vector, as described above.

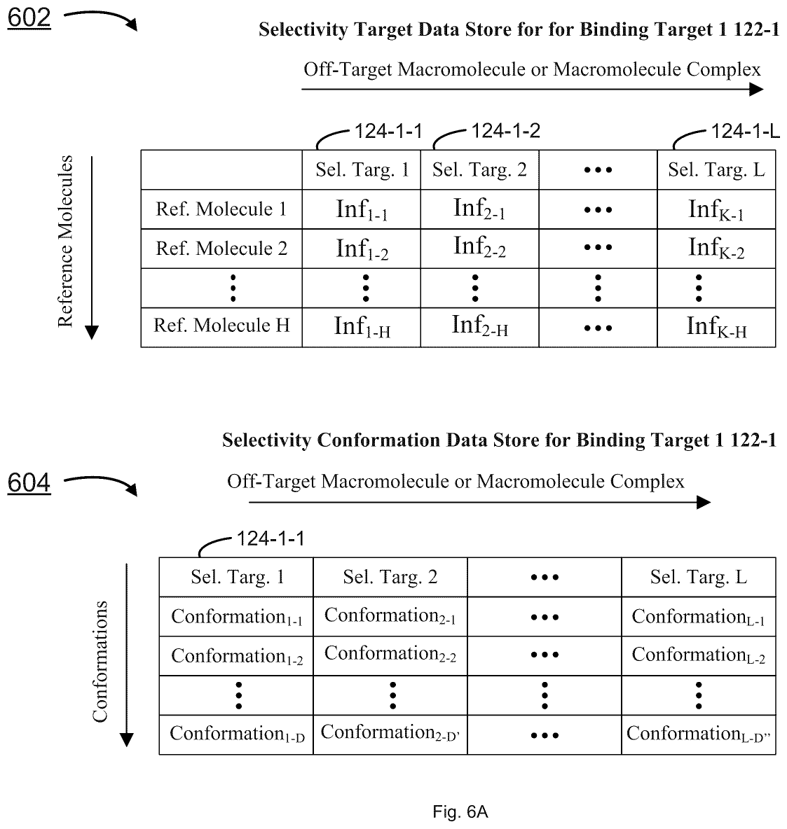

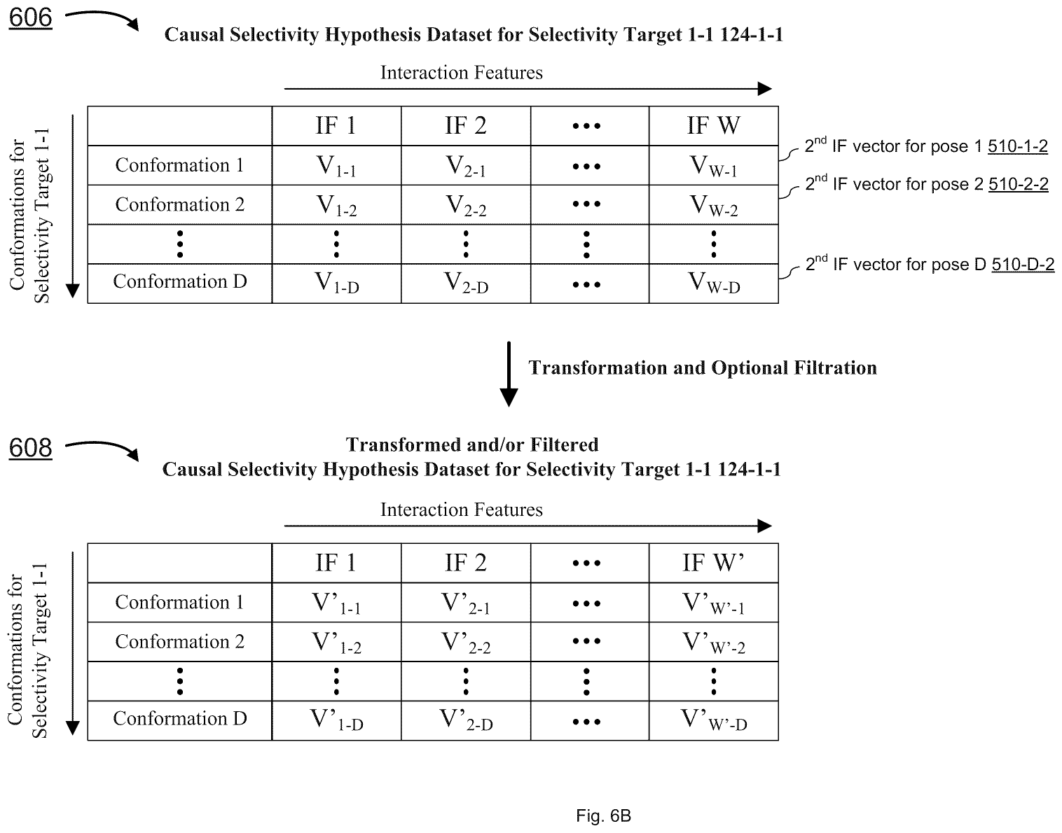

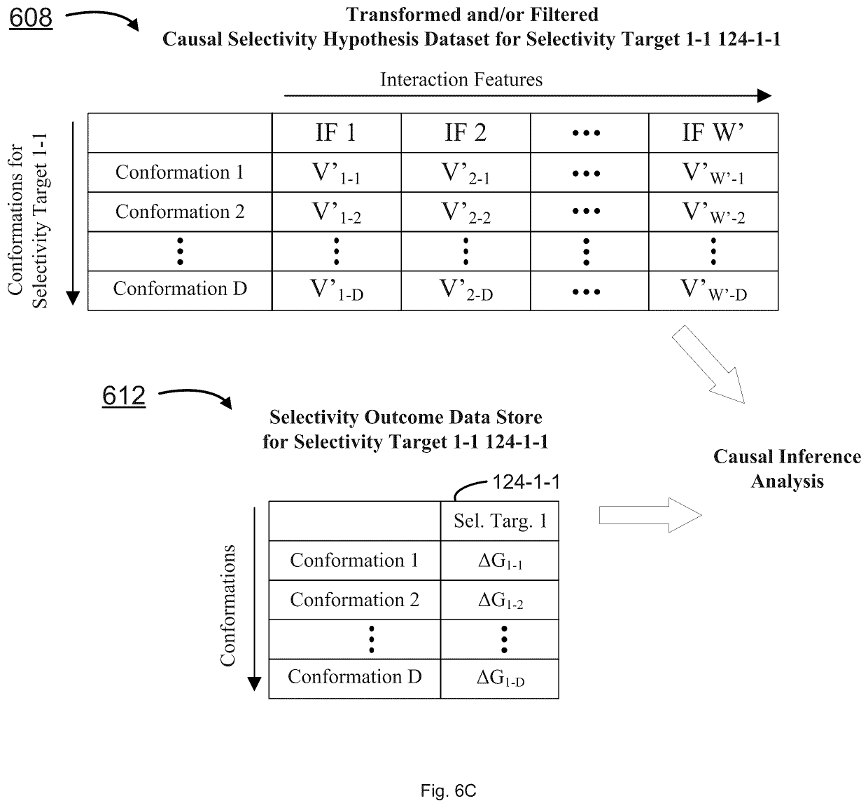

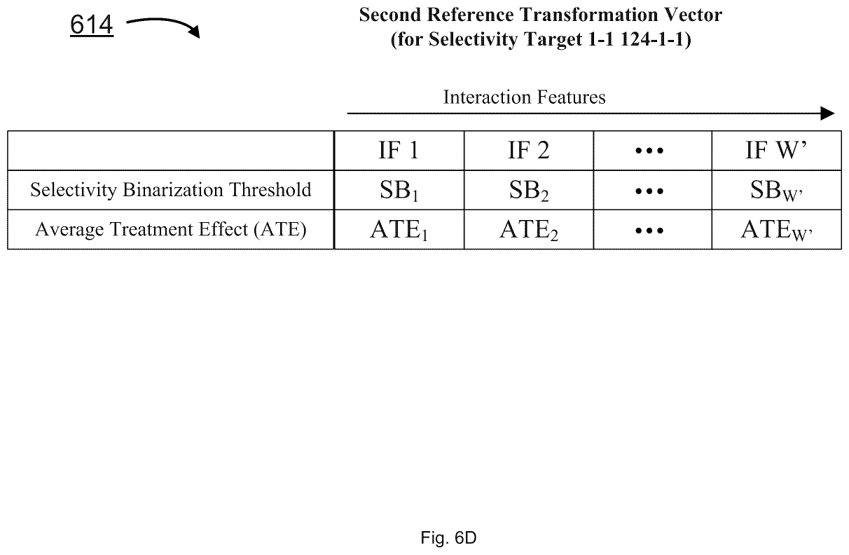

An example causal selectivity hypothesis generation process is shown as block 304 within pipeline 300 for characterizing interactions between candidate molecules and target macromolecules, depicted in FIG. 3. Causal binding hypothesis generation process 304 is further depicted as a workflow in FIG. 4B, with reference to data structures 602, 604, 606, 608, 610, 612, and 614 in FIGS. 6A-D.

In some embodiments, the causal selectivity hypothesis generation procedure includes, for each respective off-target macromolecule or macromolecule complex in a set of off-target macromolecules or macromolecule complexes, relative to the target macromolecule or macromolecule complex, for each respective reference molecule in the first plurality of reference molecules, (v) obtaining one or more corresponding three-dimensional poses of the respective reference molecule complexed to the respective off-target macromolecule or macromolecule complex, wherein each corresponding three-dimensional pose comprises a respective measure of off-target binding energy, thereby obtaining a plurality of three-dimensional poses.

In some embodiments, each respective pose comprises a respective measure of off-target binding energy (e.g., a known label) that indicates a binding specificity for the respective conformation of the reference molecule when complexed to the off-target macromolecule or macromolecule complex. In some embodiments, the respective measure of off-target binding energy is Gibbs free energy.

In some embodiments, the causal selectivity hypothesis generation procedure further includes, for each respective off-target macromolecule or macromolecule complex in a set of off-target macromolecules or macromolecule complexes, relative to the target macromolecule or macromolecule complex, for each three-dimensional pose in the plurality of three-dimensional poses, (vi) determining a second interaction feature vector for the respective pose comprising, for each respective interaction feature in a second collection of interaction features, one or more respective geometric representations and/or one or more respective attribute values for the respective interaction feature in the corresponding three-dimensional pose of the respective reference molecule complexed to the off-target macromolecule or the off-target macromolecule complex, thereby obtaining a plurality of second interaction feature vectors.

In some implementations, any of the embodiments disclosed herein for causal binding hypothesis generation is similarly contemplated for use with causal selectivity hypothesis generation, or any substitutions, modifications, additions, deletions, and/or combinations thereof, as will be apparent to one skilled in the art.

For instance, in some embodiments, the second collection of interaction features includes, none, all, or a portion of the first collection of interaction features. In some embodiments, the number of poses for a first off-target macromolecule or macromolecule complex is the same or different as the number of poses for a second off-target macromolecule or macromolecule complex, as described above. In some embodiments, a first pose in the plurality of poses comprises the same or different number of interaction features as a second pose in the plurality of poses.

In some embodiments, the causal selectivity hypothesis generation procedure further includes performing one or more filtering steps and/or one or more transformations to each respective interaction feature vector in the plurality of second interaction feature vectors.

In some embodiments, the causal selectivity hypothesis generation procedure further includes inputting each corresponding second interaction feature vector as input to a thresholding algorithm, thereby obtaining, for each respective interaction feature in the second collection of interaction features, a corresponding binary value for the respective interaction feature in the corresponding three-dimensional pose of the respective reference molecule complexed to the off-target macromolecule or the off-target macromolecule complex.

In some embodiments, the causal selectivity hypothesis generation procedure further includes (vii) responsive to inputting each respective second interaction feature vector in the plurality of second interaction feature vectors into a causal inference model, retrieving, as output from the causal inference model, for each respective interaction feature in the second collection of interaction features, a corresponding feature score for the respective interaction feature, and (viii) removing, from the respective second interaction feature vector, each respective interaction feature having a corresponding feature score that fails to satisfy a threshold feature criterion, thereby obtaining a second plurality of reference interaction features.

In some embodiments, the causal inference model is a double machine learning causal forest, and the corresponding feature score is determined as:

$$Y = {β}_{0} + D * {β}_{D} + θ X + e ,$$

where D is a first interaction feature in the second collection of interaction features, X is each respective interaction feature, other than the first interaction feature, in the second collection of interaction features, Y is a difference between (i) the respective measure of on-target binding energy for the three-dimensional pose of the respective reference molecule complexed to the target macromolecule or the target macromolecule complex and (ii) the respective measure of off-target binding energy for the three-dimensional pose of the respective reference molecule complexed to the off-target macromolecule or the off-target macromolecule complex, and the corresponding feature score is an average treatment effect.

In some non-limiting embodiments, Y is a difference between (i) a measure of central tendency (e.g., mean, median, mode, weighted mean, weighted median, and/or weighted mode) over each respective measure of on-target binding energy for each three-dimensional pose of the respective reference molecule complexed to the target macromolecule or the target macromolecule complex and (ii) a measure of central tendency over each respective measure of off-target binding energy for each three-dimensional pose of the respective reference molecule complexed to the off-target macromolecule or the off-target macromolecule complex.

For example, in some embodiments, delta G for a respective reference molecule or a respective pose thereof is calculated by combining (e.g., averaging) the Gibbs free energy of a first set of poses for a respective reference molecule with a target macromolecule, combining (e.g., averaging) the Gibbs free energy of a second set of poses for the respective reference molecule with an off-target macromolecule, and determining the difference of the averaged values.

In some embodiments, the corresponding feature score fails to satisfy a threshold feature criterion when the corresponding feature score is greater than zero.

Alternatively or additionally, in some embodiments, the causal inference model comprises propensity score matching (PSM), as disclosed elsewhere herein (see, e.g., the section entitled “Causal binding hypothesis generation,” above).

In some embodiments, the causal selectivity hypothesis generation procedure further includes performing one or more validation steps.

In some embodiments, the second plurality of reference interaction features is further used to generate the second reference transformation vector that includes, for each respective reference interaction feature in the second plurality of reference interaction features, a corresponding binarization threshold for the respective reference interaction feature. In some embodiments, the reference selectivity dataset further includes, for each respective interaction feature in the reference selectivity dataset, the corresponding ATE for the respective interaction feature determined using the causal inference model.

### Evaluating Molecules Via Causal Hypothesis.

Returning again to FIGS. 2A-J, referring to block 235, the method 200 further includes C) performing a second filtering step for the plurality of candidate molecules 132. The second filtering step comprises (i) for each respective candidate molecule 132 in the plurality of candidate molecules, determining a respective third plurality of interaction features 154-3 or a respective fourth plurality of interaction features 154-4 for the respective candidate molecule 132. Each respective interaction feature in the third plurality of interaction features 154-3 is associated with a binding affinity between the respective candidate molecule 132 and the target macromolecule or the target macromolecule complex 122, and each respective interaction feature in the fourth plurality of interaction features 154-4 is associated with a binding specificity between the respective candidate molecule 132 and the target macromolecule or the target macromolecule complex 122. The second filtering step further includes (ii) removing one or more candidate molecules 132 from the plurality of candidate molecules based at least on a count of interaction features 138, in one or both of the respective third plurality of interaction features 154-3 and the respective fourth plurality of interaction features 154-4, for each respective candidate molecule 132 in the plurality of candidate molecules. In some embodiments, the second filtering step does not include removing one or more candidate molecules from the plurality of candidate molecules. See, for example, steps D11-D12 in FIG. 4D and blocks 822-824 in FIG. 8C.

In some embodiments, the third plurality of interaction features is obtained using the causal binding hypothesis generation method disclosed above, or any embodiments thereof, as will be apparent to one skilled in the art.

Referring to block 236, in some embodiments, the method further includes, prior to the performing C): (i) obtaining a corresponding three-dimensional pose of the respective candidate molecule complexed to the target macromolecule or the target macromolecule complex, where the corresponding three-dimensional pose comprises a respective measure of on-target binding energy; (ii) determining a first interaction feature vector for the respective candidate molecule comprising, for each respective interaction feature in a first collection of interaction features, a respective geometric representation (and/or a respective attribute value) of the respective interaction feature in the corresponding three-dimensional pose of the respective candidate molecule complexed to the target macromolecule or the target macromolecule complex; (iii) responsive to inputting the first interaction feature vector into a causal inference model, retrieving, as output from the causal inference model, for each respective interaction feature in the first collection of interaction features, a corresponding feature score for the respective interaction feature; and (iv) removing, from the first interaction feature vector for the respective candidate molecule, each respective interaction feature having a corresponding feature score that fails to satisfy a threshold feature criterion, thereby obtaining the third plurality of interaction features.

In some embodiments, the corresponding three-dimensional pose of the respective candidate molecule is selected from a set of poses for the respective candidate molecule. In some embodiments, the corresponding three-dimensional pose is selected based on a docking program, as described elsewhere herein (see, e.g., the section entitled “Definitions: Pose,” above). In some implementations, the corresponding three-dimensional pose is selected as the best pose from a set of possible three-dimensional poses for the respective candidate molecule (e.g., based on a measure of binding affinity and/or specificity associated with the respective pose).

In some embodiments, the determining a first interaction feature vector for the respective candidate molecule comprises determining a set of interaction feature vectors for the respective candidate molecule, each respective interaction feature vector corresponding to a respective pose in a set of poses for the candidate molecule.

Referring to block 238, in some embodiments, the causal inference model is a double machine learning causal forest, and the corresponding feature score is determined as:

$$Y = {β}_{0} + D * {β}_{D} + θ X + e ,$$

where D is a first interaction feature in the first collection of interaction features, X is each respective interaction feature, other than the first interaction feature, in the first collection of interaction features, Y is the respective measure of on-target binding energy for the three-dimensional pose of the respective candidate molecule complexed to the target macromolecule or the target macromolecule complex, and the corresponding feature score is an average treatment effect.

Alternatively or additionally, in some embodiments, the causal inference model comprises propensity score matching (PSM), as disclosed elsewhere herein (see, e.g., the section entitled “Causal binding hypothesis generation,” above).

In some embodiments, the fourth plurality of interaction features is obtained using the causal selectivity hypothesis generation method disclosed above, or any embodiments thereof, as will be apparent to one skilled in the art.

Referring to block 240, in some embodiments, the method further includes, for each respective off-target macromolecule or off-target macromolecule complex in a set of off-target macromolecules or off-target macromolecule complexes: (v) obtaining a corresponding three-dimensional pose of the respective candidate molecule complexed to the respective off-target macromolecule or the off-target macromolecule complex, wherein the corresponding three-dimensional pose comprises a respective measure of off-target binding energy; (vi) determining a second interaction feature vector for the respective candidate molecule comprising, for each respective interaction feature in a second collection of interaction features, a respective geometric representation (and/or a respective attribute value) of the respective interaction feature in the corresponding three-dimensional pose of the respective candidate molecule complexed to the off-target macromolecule or the off-target macromolecule complex; (vii) responsive to inputting the second interaction feature vector into a causal inference model, retrieving, as output from the causal inference model, for each respective interaction feature in the second collection of interaction features, a corresponding feature score for the respective interaction feature; and (viii) removing, from the second interaction feature vector for the respective candidate molecule, each respective interaction feature having a corresponding feature score that fails to satisfy a threshold feature criterion, thereby obtaining the fourth plurality of interaction features.

In some embodiments, the corresponding three-dimensional pose of the respective candidate molecule is selected from a set of poses for the respective candidate molecule. In some embodiments, the corresponding three-dimensional pose is selected based on a docking program, as described elsewhere herein (see, e.g., the section entitled “Definitions: Pose,” above). In some implementations, the corresponding three-dimensional pose is selected as the best pose from a set of possible three-dimensional poses for the respective candidate molecule (e.g., based on a measure of binding affinity and/or specificity associated with the respective pose).

In some embodiments, the determining a second interaction feature vector for the respective candidate molecule comprises determining a set of interaction feature vectors for the respective candidate molecule, each respective interaction feature vector corresponding to a respective pose in a set of poses for the candidate molecule.

In some embodiments, the causal inference model is a double machine learning causal forest, and the corresponding feature score is determined as:

$$Y = {β}_{0} + D * {β}_{D} + θ X + e ,$$

where D is a first interaction feature in the second collection of interaction features, X is each respective interaction feature, other than the first interaction feature, in the second collection of interaction features, Y is a difference between (i) the respective measure of on-target binding energy for the three-dimensional pose of the respective candidate molecule complexed to the target macromolecule or the target macromolecule complex and (ii) the respective measure of off-target binding energy for the three-dimensional pose of the respective candidate molecule complexed to the off-target macromolecule or the off-target macromolecule complex, and the corresponding feature score is an average treatment effect.

Alternatively or additionally, in some embodiments, the causal inference model comprises propensity score matching (PSM), as disclosed elsewhere herein (see, e.g., the section entitled “Causal binding hypothesis generation,” above).

Referring to block 244, in some embodiments, the method further includes, prior to the inputting (iii) or inputting (vii), inputting the corresponding interaction feature vector as input to a thresholding algorithm, thereby obtaining, for each respective interaction feature in the first collection of interaction features, a corresponding binary value for the respective interaction feature in the corresponding three-dimensional pose of the respective candidate molecule complexed to the target macromolecule or the target macromolecule complex. In some embodiments, the thresholding algorithm is binary cross-entropy.

Referring to block 242, in some embodiments, the corresponding feature score fails to satisfy a threshold feature criterion when the corresponding feature score is greater than zero. Referring to block 246, in some embodiments, the count of interaction features is a weighted count.

### Obtaining Predictions.

Returning again to FIGS. 2A-J, referring to block 248, the method 200 further includes D) determining, for each respective candidate molecule 132 in the plurality of candidate molecules, a corresponding prediction of interaction 140 between the respective candidate molecule 132 and the target macromolecule or the target macromolecule complex 122, where the prediction 140 is obtained using at least the third plurality of interaction features 154-3 or the fourth plurality of interaction features 154-4 corresponding to the respective candidate molecule 132.

In some embodiments, the corresponding prediction of interaction includes any of the causal interaction feature scores disclosed herein, or any substitutions, modifications, additions, deletions, and/or combinations thereof, as will be apparent to one skilled in the art.

Referring to block 250, in some embodiments, the corresponding prediction of interaction between the respective candidate molecule and the target macromolecule or the target macromolecule complex is an individual treatment effect obtained as a dot product between (i) the third plurality of interaction features and (ii) for each respective interaction feature in the third plurality of interaction features, the corresponding feature score outputted by the causal inference model (e.g., via the causal binding hypothesis).

Alternatively or additionally, in some embodiments, the corresponding prediction of interaction between the respective candidate molecule and the target macromolecule or the target macromolecule complex is an individual treatment effect obtained as a dot product between (i) the fourth plurality of interaction features and (ii) for each respective interaction feature in the fourth plurality of interaction features, the corresponding feature score outputted by the causal inference model (e.g., via the causal selectivity hypothesis).

Referring to block 252, in some embodiments, the interaction between the candidate molecule and the target macromolecule or the target macromolecule complex is selected from the group consisting of a binding affinity, a binding specificity, and a measure of activity (e.g., an ADME property).

As described above, and without being limited to any one theory of operation, in some implementations, candidate molecules with individual treatment effects of less than zero are deemed to interact with the target macromolecule or macromolecule complex, due to the observation that negative values of G are associated with stronger binding affinity. Thus, for instance, in some embodiments, candidate molecules with individual treatment effects of less than zero are selected for further analysis, including but not limited to molecular dynamics simulation, synthesis, and/or lead optimization.

Referring to block 254, in some embodiments, the method further includes ranking the plurality of candidate molecules using, for each respective candidate molecule in the plurality of candidate molecules, the corresponding prediction of interaction between the respective candidate molecule and the target macromolecule or the target macromolecule complex.

Referring to block 256, in some embodiments, the method further includes filtering the plurality of candidate molecules using, for each respective candidate molecule in the plurality of candidate molecules, the corresponding prediction of interaction between the respective candidate molecule and the target macromolecule or the target macromolecule complex.

Referring to block 258, in some embodiments, the method further includes E) validating each respective candidate molecule in the plurality of candidate molecules using a molecular dynamics simulation of the respective candidate molecular and the target macromolecule or the target macromolecule complex (see, e.g., step D13 in FIG. 4D and block 826 in FIG. 8C).

Molecular dynamics simulations capture the behavior of proteins and other biomolecules in full atomic detail and at very fine temporal resolution. Such simulations can be used to decipher the functional mechanisms of proteins and other biomolecules, uncover the structural basis for disease, and aid in the design and optimization of small molecules, peptides, and proteins. See, for example, Durrant J D, McCammon J A. Molecular dynamics simulations and drug discovery. BMC Biology. 2011; 9(1):71; and Hollingsworth S A, Dror R O. Molecular dynamics simulation for all. Neuron. 2018; 99(6):1129-1143, each of which is hereby incorporated herein by reference in its entirety.

In some embodiments, a molecular dynamics simulation is performed at any one or more steps in the present disclosure. For instance, in some embodiments, a molecular dynamics simulation is performed for one or more candidate molecules during the selecting A), performing B), performing C), or determining D). In some embodiments, the molecular dynamics simulation is performed after the determining D). In some implementations, such a strategy is advantageous in that it reserves the high computational demands of the simulation for a filtered and reduced set of candidate molecules that have passed one or more criterion values (e.g., for a respective first score, a respective second score, a respective interaction count, and/or a respective interaction prediction). As an example, a one-microsecond simulation of a relatively small system (e.g., approximately 25,000 atoms) running on 24 processors can take several months to complete, limiting the number of simulations that can be feasibly and efficiently performed. See, for example, Durrant J D, McCammon J A. Molecular dynamics simulations and drug discovery. BMC Biology. 2011; 9(1):71.

In some embodiments, one or more compounds identified using the systems and methods of the present are synthesized and tested in a wet lab assay to determine whether they have potency against a therapeutic target. In some embodiments, a goal of such an assay is to determine a binding coefficient of the compound to a target polymer. In some such embodiments the binding coefficient is an IC₅₀, EC₅₀, Kd, KI, or pKI for the compound with respect to the target polymer. IC₅₀, EC₅₀, Kd, KI, and pKI, as well as suitable wet lab assays are generally described in Huser ed., 2006, High-Throughput-Screening in Drug Discovery, Methods and Principles in Medicinal Chemistry 35; and Chen ed., 2019, A Practical Guide to Assay Development and High-Throughput Screening in Drug Discovery, each of which is hereby incorporated by reference.

In some embodiments, the therapeutic target is associated with a condition. In some embodiments, the condition is a disease. In some embodiments, the condition is a cancer, hematologic disorder, autoimmune disease, inflammatory disease, immunological disorder, metabolic disorder, neurological disorder, genetic disorder, psychiatric disorder, gastroenterological disorder, renal disorder, cardiovascular disorder, dermatological disorder, respiratory disorder, viral infection, or other disease or disorder.

In some embodiments the wet lab assay test validates a compound identified by the systems and methods of the present disclosure as being a suitable compound for alleviation of the condition. In some such embodiments the compound is used in in vivo assays such as animal models.

In some embodiments, a compound identified by the systems and methods of the present disclosure is combined with one or more excipient and/or one or more pharmaceutically acceptable carrier and/or one or more diluent when administering to an animal model or a human.

Such excipients and/or carriers include all conventional solvents, dispersion media, fillers, solid carriers, coatings, antifungal and antibacterial agents, dermal penetration agents, surfactants, isotonic and absorption agents and the like.

An exemplary carrier is pharmaceutically “acceptable” in the sense of being compatible with the other ingredients of the composition (e.g., the composition comprising the selected compound in the plurality of compounds) and not injurious to a subject. The compound may conveniently be presented in unit dosage form and may be prepared by any methods well known in the art of pharmacy. Such methods include the step of bringing into association the compound with the carrier that constitutes one or more accessory ingredients. In general, the compound is prepared by uniformly and intimately bringing into association the compound with liquid carriers or finely divided solid carriers or both.

Exemplary compounds formulated for intravenous, intramuscular or intraperitoneal administration, or a pharmaceutically acceptable salt, solvate or prodrug thereof may be administered by injection or infusion.

In some embodiments, injectables for such use are prepared in conventional forms, either as a liquid solution or suspension or in a solid form suitable for preparation as a solution or suspension in a liquid prior to injection, or as an emulsion. In some embodiments, carriers include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

In some embodiments, the compound is also suitable for oral administration and presented as discrete units such as capsules, sachets or tablets each containing a predetermined amount of the test chemical compound; as a powder or granules; as a solution or a suspension in an aqueous or non-aqueous liquid; or as an oil-in-water liquid emulsion or a water-in-oil liquid emulsion. In some embodiments, the compound is presented as a bolus, electuary or paste.

In some embodiments, a tablet of the compound is made by compression or molding, optionally with one or more accessory ingredients. In some embodiments, compressed tablets are prepared by compressing in a suitable machine the test chemical compound in a free-flowing form such as a powder or granules, optionally mixed with a binder [e.g., inert diluent, preservative disintegrant (e.g. sodium starch glycolate, cross-linked polyvinyl pyrrolidone, cross-linked sodium carboxymethyl cellulose) surface-active or dispersing agent]. In some embodiments, molded tablets are made by molding in a suitable machine a mixture of the powdered compound moistened with an inert liquid diluent. In some embodiments, the tablets are optionally coated or scored and may be formulated so as to provide slow or controlled release of the compound therein using, for example, hydroxypropylmethyl cellulose in varying proportions to provide the desired release profile. In some embodiments, tablets are optionally provided with an enteric coating, to provide release in parts of the gut other than the stomach.

In some embodiments, the compound is suitable for topical administration in the mouth including lozenges comprising the active ingredient in a flavored base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

In some embodiments, the compound is suitable for topical administration to the skin. In some such instances, the compound is dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. In some embodiments, transdermal patches are used to administer the compound.

In some embodiments, the compound is suitable for parenteral administration. In such embodiments, the compound includes aqueous and non-aqueous isotonic sterile injection solutions that contain anti-oxidants, buffers, bactericides and solutes that render the compound isotonic with the blood of the intended recipient; and aqueous and non-aqueous sterile suspensions that include suspending agents and thickening agents. In some embodiments, the compound is presented in unit-dose or multi-dose sealed containers, for example, ampoules and vials, and stored in a freeze-dried (lyophilized) condition requiring only the addition of the sterile liquid carrier, for example water for injections, immediately prior to use. In some embodiments, extemporaneous injection solutions and suspensions are prepared from sterile powders, granules and tablets of the kind previously described.

It should be understood that in addition to the compound particularly mentioned above, the composition or combination of this present disclosure (e.g., the compound selected from the plurality of compounds) may include other agents conventional in the art having regard to the type of composition or combination in question, for example, those suitable for oral administration may include such further agents as binders, sweeteners, thickeners, flavoring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

In some embodiments, the present disclosure informs the selection of one or more human subjects for treatment with the compound and/or selection of one or more human subjects for continuation or discontinuation of treatment with the compound.

In some embodiments, the present disclosure informs the dosing amount, duration, and/or frequency of the compound in one or more human subjects for treatment.

In some embodiments, the present disclosure informs the design of a clinical trial, the clinical trial comprising the use of the compound. In some embodiments, the present disclosure informs the design of an adaptive clinical trial, the adaptive clinical trial comprising the use of the compound.

In some embodiments, the present disclosure further comprises formulating the compound for use in a therapy. In some embodiments, this includes formulating the compound with any of the excipients, pharmaceutically acceptable carrier, diluents, or other pharmacological formulations described in the present disclosure or known in the art. In some embodiments, the therapy is to alleviate a condition such as inflammation. In some embodiments the therapy is to alleviate or treat a disease or disorder. In some embodiments the disease or disorder is cancer, a hematologic disorder, an autoimmune disease, an inflammatory disease, an immunological disorder, a metabolic disorder, a neurological disorder, a genetic disorder, a psychiatric disorder, a gastroenterological disorder, a renal disorder, a cardiovascular disorder, a dermatological disorder, a respiratory disorder, a viral infection, or other disease or disorder.

Use cases. In some embodiments, the systems and methods disclosed herein are advantageously used in any number of applications, including but not limited to hit discovery, hit-to-lead discovery, lead optimization, off-target side-effect prediction, molecular dynamics simulations, toxicity prediction, potency optimization, selectivity optimization, fitness modeling, drug repurposing, drug resistance prediction, personalized medicine, drug trial design, agrochemical design, and/or materials science.

### Further Embodiments

Another aspect of the present disclosure provides a computer system comprising: one or more processors; memory; and one or more programs, where the one or more programs are stored in the memory and are configured to be executed by the one or more processors, the one or more programs for characterizing an interaction between a candidate molecule and a target macromolecule or a target macromolecule complex, the one or more programs including instructions for: A) selecting a plurality of candidate molecules from a collection of candidate molecules based on a respective first score for the interaction between each respective candidate molecule in the collection of candidate molecules and the target macromolecule or the target macromolecule complex, wherein the plurality of candidate molecules comprises at least 1×10⁶ candidate molecules; B) performing a first filtering step for the plurality of candidate molecules comprising: for each respective candidate molecule in the plurality of candidate molecules: responsive to inputting a two-dimensional molecular graph of the respective candidate molecule into a first model, retrieving, as output from the first model, a corresponding first plurality of interaction features for a complex formed between the respective candidate molecule and the target macromolecule or the target macromolecule complex, responsive to inputting the two-dimensional molecular graph of the respective candidate molecule into a second model, retrieving, as output from the second model, a corresponding second plurality of interaction features for a complex formed between the respective candidate molecule and an off-target macromolecule or off-target macromolecule complex, other than the target macromolecule or target macromolecule complex, and using at least the first plurality of interaction features or the second plurality of interaction features to obtain a corresponding second score for the interaction between the respective candidate molecule and the target macromolecule or the target macromolecule complex, and removing one or more candidate molecules from the plurality of candidate molecules based on an evaluation of the corresponding second score for each respective candidate molecule in the plurality of candidate molecules, where the first model comprises a first plurality of at least 1000 parameters and the second model comprises a second plurality of at least 1000 parameters; C) performing a second filtering step for the plurality of candidate molecules comprising: (i) for each respective candidate molecule in the plurality of candidate molecules: determining a respective third plurality of interaction features or a respective fourth plurality of interaction features for the respective candidate molecule, where: each respective interaction feature in the third plurality of interaction features is associated with a binding affinity between the respective candidate molecule and the target macromolecule or the target macromolecule complex, and each respective interaction feature in the fourth plurality of interaction features is associated with a binding specificity between the respective candidate molecule and the target macromolecule or the target macromolecule complex, and (ii) removing one or more candidate molecules from the plurality of candidate molecules based at least on a count of interaction features, in one or both of the respective third plurality of interaction features and the respective fourth plurality of interaction features, for each respective candidate molecule in the plurality of candidate molecules; and D) determining, for each respective candidate molecule in the plurality of candidate molecules, a corresponding prediction of interaction between the respective candidate molecule and the target macromolecule or the target macromolecule complex, wherein the prediction is obtained using at least the third plurality of interaction features or the fourth plurality of interaction features corresponding to the respective candidate molecule.

Another aspect of the present disclosure provides a computer readable storage medium storing one or more programs, the one or more programs comprising instructions, which when executed by an electronic device with one or more processors and a memory cause the electronic device to characterize an interaction between a candidate molecule and a target macromolecule or a target macromolecule complex by a method comprising: A) selecting a plurality of candidate molecules from a collection of candidate molecules based on a respective first score for the interaction between each respective candidate molecule in the collection of candidate molecules and the target macromolecule or the target macromolecule complex, wherein the plurality of candidate molecules comprises at least 1×10⁶ candidate molecules; B) performing a first filtering step for the plurality of candidate molecules comprising: for each respective candidate molecule in the plurality of candidate molecules: responsive to inputting a two-dimensional molecular graph of the respective candidate molecule into a first model, retrieving, as output from the first model, a corresponding first plurality of interaction features for a complex formed between the respective candidate molecule and the target macromolecule or the target macromolecule complex, responsive to inputting the two-dimensional molecular graph of the respective candidate molecule into a second model, retrieving, as output from the second model, a corresponding second plurality of interaction features for a complex formed between the respective candidate molecule and an off-target macromolecule or off-target macromolecule complex, other than the target macromolecule or the target macromolecule complex, and using at least the first plurality of interaction features or the second plurality of interaction features to obtain a corresponding second score for the interaction between the respective candidate molecule and the target macromolecule or the target macromolecule complex, and removing one or more candidate molecules from the plurality of candidate molecules based on an evaluation of the corresponding second score for each respective candidate molecule in the plurality of candidate molecules, where the first model comprises a first plurality of at least 1000 parameters and the second model comprises a second plurality of at least 1000 parameters; C) performing a second filtering step for the plurality of candidate molecules comprising: (i) for each respective candidate molecule in the plurality of candidate molecules: determining a respective third plurality of interaction features or a respective fourth plurality of interaction features for the respective candidate molecule, wherein: each respective interaction feature in the third plurality of interaction features is associated with a binding affinity between the respective candidate molecule and the target macromolecule or the target macromolecule complex, and each respective interaction feature in the fourth plurality of interaction features is associated with a binding specificity between the respective candidate molecule and the target macromolecule or the target macromolecule complex, and (ii) removing one or more candidate molecules from the plurality of candidate molecules based at least on a count of interaction features, in one or both of the respective third plurality of interaction features and the respective fourth plurality of interaction features, for each respective candidate molecule in the plurality of candidate molecules; and D) determining, for each respective candidate molecule in the plurality of candidate molecules, a corresponding prediction of interaction between the respective candidate molecule and the target macromolecule or the target macromolecule complex, wherein the prediction is obtained using at least the third plurality of interaction features or the fourth plurality of interaction features corresponding to the respective candidate molecule.

Still another aspect of the present disclosure provides a method for identifying a candidate molecule having a target activity with a target macromolecule or a target macromolecule complex, the method comprising: A) obtaining a plurality of molecular reactions and a plurality of at least 1×10⁶ molecular components; B) performing a procedure comprising: i) obtaining, for each respective molecular component in the set of molecular components, a respective transformation of the respective molecular component that represents a corresponding one or more molecular reactions in the plurality of molecular reactions, thereby generating a plurality of molecular intermediates; ii) removing, from the plurality of molecular intermediates, one or more respective molecular intermediates based on a respective first score for a binding interaction between each respective molecular intermediate in the plurality of molecular intermediates and the target macromolecule or the target macromolecule complex, where: for each respective molecular intermediate in the plurality of molecular intermediates, the respective first score is obtained using at least a corresponding first plurality of interaction features for a complex formed between the respective molecular intermediate and the target macromolecule or target macromolecule complex; iii) assigning, after the removing, the plurality of molecular intermediates to the plurality of molecular components; iv) repeating the obtaining i), removing ii), and assigning iii) until a respective second score for the binding interaction between each respective molecular intermediate in the plurality of molecular intermediates and the target macromolecule or target macromolecule complex satisfies a threshold exit criterion, where: for each respective molecular intermediate in the plurality of molecular intermediates, the respective second score is obtained using at least a corresponding second plurality of interaction features for a complex formed between the respective molecular intermediate and the target macromolecule or target macromolecule complex; and v) generating a collection of candidate molecules using the plurality of molecular intermediates, wherein the plurality of candidate molecules comprises at least 1×10⁶ candidate molecules; and C) determining, for each respective candidate molecule in the plurality of candidate molecules, a corresponding prediction of the target activity between the respective candidate molecule and the target macromolecule or target macromolecule complex.

In some embodiments, the determining the target activity includes determining a binding affinity, a binding specificity, and/or a measure of activity (e.g., an ADME property).

In some embodiments, the determining the target activity comprises performing a molecular dynamics simulation of the respective candidate molecular and the target macromolecule or the target macromolecule complex.

### EXAMPLES

### Example 1—Next-Generation Libraries for the Discovery of Novel, Brain Penetrant and Selective Kinase Inhibitors

WEE1 is a G2 checkpoint kinase, inhibition of which causes preferential cancer death (e.g., mitotic catastrophe). WEE1 inhibitors have efficacy against solid tumors, the best indication of which is found in glioblastoma, in which WEE1 is the most overexpressed kinase and has the strongest correlation with patient survival inhibition. Current WEE1 inhibitors are only being tested in solid tumors outside the brain.

Conventional therapeutics approaches often fail to solve selectivity challenges with traditional medicinal chemistry. For example, previous reports indicated that the major obstacles preventing the success of drug discovery efforts for putative WEE1 inhibitors (e.g., adavosertib, AZD1775) included kinome selectivity and tolerability profiles, such as off-target activity for Polo-like kinase (Plk)1, despite evidence that WEE1 is an important target in cancer.

Brain penetration is a particular challenge, with leading selective WEE1 inhibitors in clinical studies demonstrating encouraging efficacy against peripheral tumors but poor brain exposure. These studies reported low IC₅₀ for WEE1 in vitro indicative of excellent potency for the compounds tested, but considerably poor in vitro and in vivo brain penetration as measured using MDCK-MDR1 efflux ratio and Kpuu. MDCK-MDR1 is an assay that measures brain permeability and export of compounds by P-glycoprotein. Kpuu measures the ratio of free concentration of drug in plasma and brain.

### Prediction of Interaction and Generation of Interaction Feature Scores.

A model was constructed to predict candidate molecules having binding affinity to a target macromolecule, specifically in vivo brain penetration with binding affinity to WEE1. In particular, the model was used to generate candidate molecules that demonstrated both in vivo and in vitro brain penetration. Using the candidate molecules generated from the first model, measures of IC₅₀, MDCK-MDR1 efflux ratio, Kpuu indicated excellent potency, excellent in vitro brain penetration, and excellent in vivo brain penetration for all candidate molecules tested. Moreover, the first model was found to be applicable for generating brain penetrant drugs targeting other target macromolecules other than WEE1.

Candidate molecules that exhibited both binding affinity and binding selectivity were then searched, specifically WEE1 inhibitors that were both brain-penetrant and ultra-selective. To perform this identification, a method was performed for each candidate molecule in a set of commercial libraries of candidate molecules.

Causal binding hypothesis. First, a causal binding hypothesis generation method was performed. The method included obtaining, for a respective binding target macromolecule or macromolecule complex (e.g., WEE1), information for the plurality of candidate molecules including a Gibbs free energy of each molecule when bound to the target macromolecule. A plurality of conformations of each molecule bound to the target were also obtained. For each conformation, a plurality of interaction features (IFs) were calculated (e.g., partial charge information, pharmacophore information, etc.), thus generating, for each respective pose in the plurality of poses, a corresponding IF vector including IFs for the interaction of the corresponding candidate molecule with the target macromolecule WEE1. The IF vectors were optionally filtered, transformed (e.g., binarized by a thresholding algorithm), and used in a debias strategy in order to refute the impact of confounder features present in each conformation, thus providing a respective score (e.g., average treatment effect) for each respective IF in the plurality of IFs that was predictive of a causal relationship to binding affinity. The transformed and/or filtered list of IFs was further used to calculate a binding interaction feature score for each conformation of each respective candidate molecule in the plurality of candidate molecules bound to the target macromolecule.

Causal selectivity hypothesis. Next, a causal selectivity hypothesis generation method was performed. The method included obtaining, for one or more selectivity targets in a set of selectivity targets for the respective binding target macromolecule (e.g., an off-target macromolecule relative to the target macromolecule WEE1), information for the plurality of candidate molecules including a Gibbs free energy of each molecule when bound to the selectivity target. Conformations of each molecule bound to the selectivity target were also obtained. For each conformation, interaction features (IFs) were calculated (e.g., partial charge information, pharmacophore information, etc.), thus generating for each respective pose in the plurality of poses, a corresponding IF vector including IFs for the interaction of the corresponding candidate molecule with the selectivity target.

The IF vectors were optionally filtered, transformed (e.g., binarized by a thresholding algorithm), and used in a debias strategy in order to refute the impact of confounder features present in each conformation, thus providing a respective score (e.g., average treatment effect) for each respective IF in the plurality of IFs that was predictive of a causal relationship to binding specificity. For each respective candidate molecule in the plurality of candidate molecules, an “outcome” or “treatment effect” used in the debias strategy included a corresponding difference (delta G) in (i) the Gibbs free energy for the respective ligand when bound to the binding target WEE1 (G_{P}) and (ii) the Gibbs free energy for the respective ligand with bound to the respective selectivity target (G_{S}) was determined. For instance, using the equation delta G=G_{P}−G_{S}, for a given candidate molecule that binds more strongly to the primary binding target than the selectivity target (G_{P}<G_{S}), the delta G was determined to be negative. The transformed and/or filtered list of IFs was further used to calculate a specificity interaction feature score for each conformation of each respective candidate molecule in the plurality of candidate molecules bound to the target and/or the off-target macromolecule.

Binding graph neural network. In another approach to better predict binding affinity (e.g., potency) of a candidate molecule to a target macromolecule (e.g., WEE1), a first graph neural network was obtained. First, a plurality of training ligands was obtained, where each training ligand in the plurality of training ligands binds to the binding target macromolecule or macromolecule complex. A 2D molecular graph for each training ligand was also obtained. For each training ligand, a corresponding conformation of a complex formed by the target and the training ligand was obtained and used via the causal binding hypothesis method above to generate, for each respective training conformation, a corresponding measured IF vector. For each conformation corresponding to a respective training ligand in the plurality of training ligands, the 2D molecular graph was used as input and the corresponding measured IF vector was used as a true label for backpropagation, thus training the first graph neural network. Use of the trained first graph neural network included inputting a 2D graph of a candidate molecule for prediction of binding affinity and receiving, as output, a corresponding predicted IF vector that included IFs for the interaction of the corresponding candidate molecule with the binding target. The IF vector was further used to calculate a binding interaction feature score for the respective conformation.

Selectivity graph neural network. A similar method was performed as above, via the causal selectivity hypothesis method, to obtain a trained second graph neural network. Use of the trained second graph neural network included inputting a 2D graph of a candidate molecule for prediction of binding specificity and receiving, as output, a corresponding predicted IF vector that included IFs for the interaction of the corresponding candidate molecule with the specificity target. The IF vector was further used to calculate a specificity interaction feature score for the respective conformation.

ADME and other models. Candidate molecules were further used in various models for predicting physicochemical properties and/or drug-likeness properties, among others (e.g., ADME-Tox models). An aggregated score (e.g., normalized score) was then obtained using at least the binding affinity (e.g., potency), selectivity, and optional ADME model scores.

### Results.

Very few compounds in the commercial libraries tested were able to achieve the target candidate profiles (TCP) established in the assay with respect to their interaction with the target macromolecule WEE1. Target candidate profiles refer to a list of all the target requirements of a preclinical drug candidate. For instance, a TCP covers key features such as activity, specificity, stability, and physicochemical properties. In a particular example, no candidate molecules in the ultra-large commercial library of more than 20 billion candidate molecules were predicted using these methods to be both high-affinity binders and meeting the TCP requirements set in the assay.

### Molecular Generation.

Accordingly, a method was used to generate custom candidate molecules for prediction scoring, where the scoring used any one or more of the interaction prediction methods described above.

In an example embodiment, a plurality of more than 165 molecular reactions and a plurality of more than 1 million molecular components was obtained. For each respective molecular component in the plurality of molecular components, the respective molecular component was transformed using a corresponding one or more molecular reactions in the plurality of molecular reactions, thereby generating a plurality of molecular intermediates. For each respective molecular intermediate in the plurality of molecular intermediates, the respective first score for the interaction between the respective molecular intermediate and the target macromolecule or the target macromolecule complex was determined. Optionally, one or more molecular intermediates was removed from the plurality of molecular intermediates based on a respective first score for the interaction between each respective molecular intermediate and the target macromolecule or target macromolecule complex WEE1. The process was performed for one or more iterations, using directed or genetic algorithms to apply reactions to molecular components, or until all generated molecules satisfied a scoring criterion.

In some embodiments, candidate molecules were selected from a collection of molecular intermediates based on scores for the interaction between the molecules and a target. One or more filtering steps were performed based on causal interaction feature scores determined using any of the interaction prediction and scoring methods described above. Alternative or additionally, one or more candidate molecules were selected for further downstream validation and/or lead optimization (e.g., using molecular dynamics simulations) based on a causal interaction feature score (e.g., an individual treatment score) calculated from the IF vectors, and/or the IFs thereof, obtained via any of the interaction prediction and scoring methods described above.

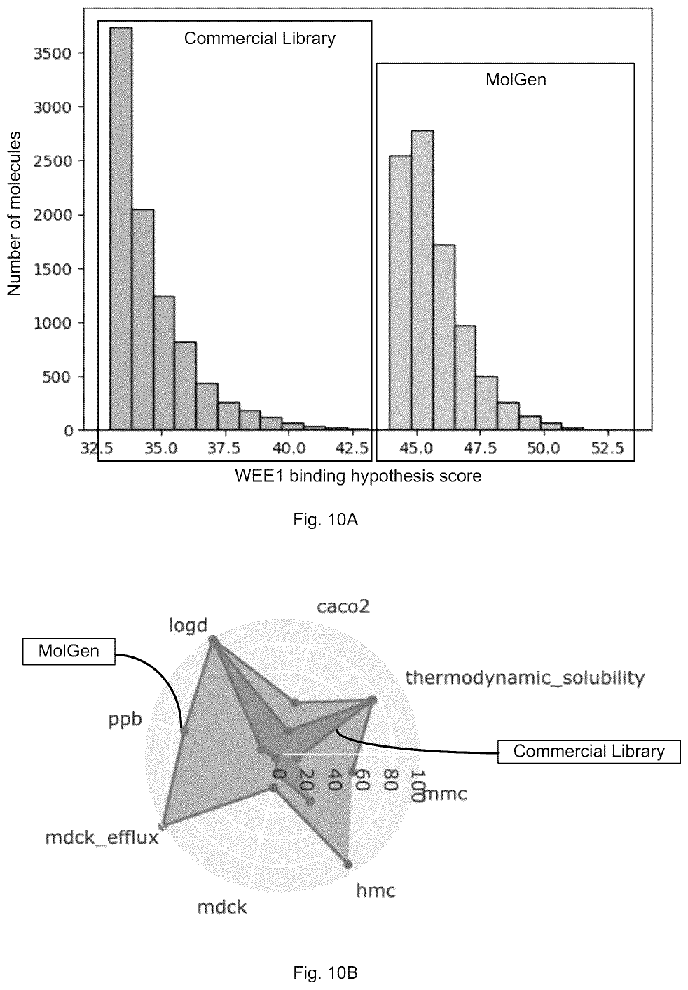

Unlike the commercial libraries tested previously, a next-generation library (“MolGen”) comprising candidate molecules optimized for WEE1 TCP and generated as disclosed herein included candidate molecules that satisfied all of the set TCP requirements. For example, as shown in FIG. 10A, the MolGen library included many more compounds with higher binding affinity (potency) compared to a commercial library, as measured using a WEE1-target binding hypothesis score. Moreover, as shown in FIG. 10B, the MolGen library included many more compounds having target pharmacological properties, compared to a commercial library, where values for the target pharmacological properties are considerably lower and exhibit lower diversity compared to the MolGen library.

A further investigation of the generated molecules identified novel, potent WEE1 inhibitors in just a single iteration of synthesis, highlighting the ability of the molecular generation method to accurately and consistently generate candidate molecules that satisfy target binding, selectivity, and/or pharmacological criteria. In other words, molecular generation method disclosed herein reduces the burden of exhaustively synthesizing and testing a large collection of low-potential or low-quality hit compounds during the hit-to-lead and lead optimization stages of drug discovery. As an illustrative example, 12 molecules identified in the MolGen method were synthesized, of which 7 were both novel and potent WEE1 inhibitors at both 30 μM and 100 nM. 4 out of the 12 had lead-candidate potency against WEE1 at 15 nm, and 6 had greater than 35× selectivity relative to the off-target macromolecule PLK1, of which at least 1 cleared a diverse kinome panel. Additionally, 4 out of the 12 demonstrated blood-brain barrier penetration in vivo (BBBP at less than 1 μM). All compounds synthesized had less than 25% similarity both to each other and to any published WEE1 inhibitors.

In contrast, a commercial library yielded considerably poorer results, with only 6 compound exhibiting binding affinity at 30 μM. The commercial library failed to meet any of the other metrics evaluated (100 nM, 15 nm, PLK1 selectivity, and BBBP at less than 1 μM). Moreover, the molecular generation method of the present disclosure was performed at a lower cost ($22K-$25K) compared to the commercial library ($25K).

Thus, the presently disclosed systems and methods improve lead compound identification at a higher hit rate, potency, selectivity, and cost-efficiency compared to conventional methods, while further reducing major bottlenecks common in drug discovery pipelines.

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1-42. (canceled)

43. A computer system comprising:
one or more processors;
memory; and
one or more programs, wherein the one or more programs are stored in the memory and are configured to be executed by the one or more processors, the one or more programs for characterizing interactions, the one or more programs including:
A) instructions for selecting a plurality of candidates from a candidate collection store based on a respective first score for the interaction between each respective candidate in the candidate collection store and a target, wherein the plurality of candidates comprises at least 1×10⁶ candidate molecules;
B) instructions for performing a first filtering step for the plurality of candidates comprising:
for each respective candidate in the plurality of candidates:
responsive to inputting a two-dimensional graph of the respective candidate into a first model, retrieving, as output from the first model, a corresponding first plurality of modeling features for an interaction between the respective candidate and the target,
responsive to inputting the two-dimensional graph of the respective candidate into a second model, retrieving, as output from the second model, a corresponding second plurality of modeling features for an interaction between the respective candidate and an off-target entity, other than the target, and
using at least the first plurality of modeling features or the second plurality of modeling features to obtain a corresponding second score for the interaction between the respective candidate and the target, and
removing one or more candidates from the plurality of candidates based on an evaluation of the corresponding second score for each respective candidate in the plurality of candidates,
wherein the first model comprises a first plurality of at least 1000 parameters and the second model comprises a second plurality of at least 1000 parameters;
C) instructions for performing a second filtering step for the plurality of candidates comprising:
(i) for each respective candidate in the plurality of candidates:
determining a respective third plurality of modeling features or a respective fourth plurality of modeling features for the respective candidate, wherein:
each respective modeling feature in the third plurality of modeling features is associated with affinity between the respective candidate and the target, and
each respective modeling feature in the fourth plurality of modeling features is associated with specificity between the respective candidate and the target, and
(ii) removing one or more candidates from the plurality of candidates based at least on a count of modeling features, in one or both of the respective third plurality of modeling features and the respective fourth plurality of modeling features, for each respective candidate in the plurality of candidates; and
D) instructions for determining, for each respective candidate in the plurality of candidates, a corresponding prediction of interaction between the respective candidate and the target, wherein the prediction is obtained using at least the third plurality of modeling features or the fourth plurality of modeling features corresponding to the respective candidate.

44-45. (canceled)

46. The method of claim 43, further comprising, prior to the selecting A):
obtaining a plurality of reactions and a plurality of components;
for each respective component in the plurality of components, transforming the respective component using a corresponding one or more reactions in the plurality of reactions, thereby generating a plurality of intermediates;
determining, for each respective intermediate in the plurality of intermediates, the respective first score for an interaction between the respective intermediate and the target; and
removing one or more intermediates from the plurality of intermediates based on the respective first score for the interaction between each respective intermediate and the target.

47. The method of claim 46, further comprising:
for each respective intermediate in the plurality of intermediates, transforming the respective intermediate using a corresponding one or more reactions in the plurality of reactions, thereby generating the candidate collection store; and
for each respective candidate in the candidate collection store, determining the respective first score for the interaction between the respective candidate and the target, and wherein:
the selecting A) comprises removing one or more candidates from the candidate collection store based on the respective first score for the interaction between each respective candidate and the target.

48. The method of claim 46, further comprising:
for each respective intermediate in the plurality of intermediates:
responsive to inputting the respective intermediate into a reinforcement learning model, retrieving, as output from the reinforcement learning model, a respective transformation of the respective intermediate, wherein the respective transformation:
(i) represents a corresponding one or more molecular reactions in the plurality of molecular reactions, and
(ii) is selected from a probability distribution of a plurality of transformations, for the respective intermediate, associated with the corresponding one or more molecular reactions, thereby generating the candidate collection store; and
for each respective candidate in the candidate collection store, determining the respective first score for the interaction between the respective candidate and the target, and wherein:
the selecting A) comprises removing one or more candidates from the candidate collection store based on the respective first score for the interaction between each respective candidate and the target.

49. The method of claim 48, wherein the reinforcement learning model comprises a third plurality of at least 1000 parameters, further comprising training the reinforcement learning model prior to the inputting by a procedure comprising, for each respective component in the plurality of components:
(i) obtaining a respective representation of a chemical structure of the respective component;
(ii) responsive to inputting the respective representation of the chemical structure of the respective component into the reinforcement learning model, retrieving, as respective training output from the reinforcement learning model, a corresponding plurality of predicted transformations, wherein each respective predicted transformation in the plurality of predicted transformations:
(a) represents a corresponding one or more molecular reactions in the plurality of molecular reactions, and
(b) corresponds to a respective intermediate in the plurality of intermediates; and
(iii) for each respective predicted transformation in the plurality of predicted transformations, using the respective first score for the interaction between the corresponding candidate for the respective predicted transformation and the target to adjust the third plurality of parameters.

50. The method of claim 47, further comprising repeating the transforming, determining, and selecting for each iteration in a plurality of iterations.

51. The method of claim 43, further comprising, prior to the selecting A), for each respective candidate in the candidate collection store:
responsive to inputting a two-dimensional graph of the respective candidate into the first model, retrieving, as output from the first model, a corresponding fifth plurality of modeling features for an interaction between the respective candidate and the target, wherein each respective modeling feature in the fifth plurality of modeling features is associated with affinity between the respective candidate and the target;
tallying the fifth plurality of modeling features for the respective candidate, thereby obtaining a corresponding modeling feature count;
tallying a number of heavy atoms in the respective candidate, thereby obtaining a corresponding heavy atom count; and
calculating the respective first score for the interaction between the respective candidate and the target as a ratio between (i) the corresponding modeling feature count and (ii) the corresponding heavy atom count; and
removing one or more candidates, from the candidate collection store, based at least on a count of modeling features in the fifth plurality of modeling features for each respective candidate in the candidate collection store.

52. The method of claim 43, wherein the first model is a first graph neural network.

53. The method of claim 43, wherein each respective modeling feature in the first plurality of modeling features is predicted by the first model to be causal for affinity between the respective candidate and the target.

54. The method of claim 43, further comprising, prior to the performing B), training the first model by a procedure comprising, for each respective training molecule in a first plurality of at least 100,000 training molecules:
(i) obtaining a corresponding three-dimensional pose of the respective training molecule complexed to the target;
(ii) determining a corresponding modeling feature vector for the respective training molecule comprising, for each respective modeling feature in a first collection of modeling features, a respective geometric representation of the respective modeling feature in the corresponding three-dimensional pose of the respective training molecule complexed to the target;
(iii) transforming the corresponding modeling feature vector using a first reference transformation vector, thereby obtaining, for each respective modeling feature in the first collection of modeling features, a corresponding reference label that indicates whether, or to what degree, the respective modeling feature is causal for affinity between the respective training molecule and the target;
(iv) obtaining a respective training two-dimensional graph of a chemical structure of the respective training molecule;
(v) responsive to inputting the respective training two-dimensional graph of the chemical structure of the respective training molecule into the first model, retrieving, as respective training output from the first model:
for each respective modeling feature in the first collection of modeling features, a corresponding training predicted label that indicates whether, or to what degree, the respective modeling feature is causal for affinity between the respective training molecule and the target;
(vi) applying a respective difference to a loss function to obtain a respective output of the loss function, wherein the respective difference is between:
for each respective modeling feature in the first collection of modeling features, (a) the corresponding training predicted label from the first model and (b) the corresponding reference label; and
(vii) using the respective output of the loss function to adjust the first plurality of parameters.

55. The method of claim 43, wherein a respective modeling feature is selected from the group consisting of: three-dimensional partial charges, three-dimensional pharmacophores, or molecular dynamics residue interaction time.

56. The method of claim 43, wherein the performing B) further comprises, for each respective candidate in the plurality of candidates:
responsive to inputting a corresponding representation of a chemical structure of the respective candidate into a third model, retrieving, as output from the third model, a corresponding measure of activity for the respective candidate; and
using the corresponding measure of activity to obtain the corresponding second score for the interaction between the respective candidate and the target.

57. The method of claim 43, wherein the first plurality of parameters comprises at least 10,000, at least 100,000, or at least 1×10⁶ parameters, and wherein the second plurality of parameters comprises at least 10,000, at least 100,000, or at least 1×10⁶ parameters.

58. The method of claim 43, further comprising, prior to the performing C):
(i) obtaining a corresponding three-dimensional pose of the respective candidate complexed to the target, wherein the corresponding three-dimensional pose comprises a respective measure of on-target binding energy;
(ii) determining a first modeling feature vector for the respective candidate comprising, for each respective modeling feature in a first collection of modeling features, a respective geometric representation of the respective modeling feature in the corresponding three-dimensional pose of the respective candidate complexed to the target;
(iii) responsive to inputting the first modeling feature vector into a causal inference model, retrieving, as output from the causal inference model, for each respective modeling feature in the first collection of modeling features, a corresponding feature score for the respective modeling feature; and
(iv) removing, from the first modeling feature vector for the respective candidate, each respective modeling feature having a corresponding feature score that fails to satisfy a threshold feature criterion, thereby obtaining the third plurality of modeling features.

59. The method of claim 58, wherein the causal inference model is a double machine learning causal forest, and the corresponding feature score is determined as:
$$Y = {β}_{0} + D * {β}_{D} + θ X + e ,$$
wherein:
D is a first modeling feature in the first collection of modeling features,
X is each respective modeling feature, other than the first modeling feature, in the first collection of modeling features,
Y is the respective measure of on-target binding energy for the three-dimensional pose of the respective candidate complexed to the target, and
the corresponding feature score is an average treatment effect.

60. The method of claim 58, further comprising, for each respective off-target entity in a set of off-target entities:
(v) obtaining a corresponding three-dimensional pose of the respective candidate complexed to the respective off-target entity, wherein the corresponding three-dimensional pose comprises a respective measure of off-target binding energy;
(vi) determining a second modeling feature vector for the respective candidate comprising, for each respective modeling feature in a second collection of modeling features, a respective geometric representation of the respective modeling feature in the corresponding three-dimensional pose of the respective candidate complexed to the off-target entity;
(vii) responsive to inputting the second modeling feature vector into a causal inference model, retrieving, as output from the causal inference model, for each respective modeling feature in the second collection of modeling features, a corresponding feature score for the respective modeling feature; and
(viii) removing, from the second modeling feature vector for the respective candidate, each respective modeling feature having a corresponding feature score that fails to satisfy a threshold feature criterion, thereby obtaining the fourth plurality of modeling features.

61. The method of claim 58, further comprising, prior to the inputting (iii), inputting the corresponding modeling feature vector as input to a thresholding algorithm, thereby obtaining, for each respective modeling feature in the first collection of modeling features, a corresponding binary value for the respective modeling feature in the corresponding three-dimensional pose of the respective candidate complexed to the target.

62. The method of claim 58, wherein the corresponding prediction of interaction between the respective candidate and the target is an individual treatment effect obtained as a dot product between (i) the third plurality of modeling features and (ii) for each respective modeling feature in the third plurality of modeling features, the corresponding feature score outputted by the causal inference model.

63. The method of claim 43, further comprising ranking or filtering the plurality of candidates using, for each respective candidate in the plurality of candidates, the corresponding prediction of interaction between the respective candidate and the target.

64. The method of claim 43, further comprising:
E) validating each respective candidate in the plurality of candidates using a molecular dynamics simulation of the respective candidate molecular and the target.

65. The method of claim 43, wherein the count of modeling features is a weighted count.

66. The method of claim 43, wherein the interaction between the candidate and the target is selected from the group consisting of affinity, specificity, and a measure of activity.
